(19) United States
(12) Patent Application Publication
(10) Pub. No.: US 2019/0133930 A1
(43) Pub. Date: May 9, 2019

(54) Methods of Anesthetizing Nerve Tissue in the Trigeminal Nerve Pathway and Medical Uses Thereof

(71) Applicant: Apollonia, LLC, Fort Collins, CO (US)

(72) Inventor: Mark Mulvahill, St. Louis Park, MN (US)

(73) Assignee: Apollonia, LLC, Fort Collins, CO (US)

(21) Appl. No.: 16/234,926

(22) Filed: Dec. 28, 2018

Related U.S. Application Data
(63) Continuation of application No. 15/664,972, filed on Jul. 31, 2017, now abandoned.
Application No. 15/664,972 is a continuation of application No. 14/741,330, filed on Jun. 16, 2015, now abandoned.
(60) Provisional application No. 62/012,946, filed on Jun. 16, 2014.
(60) Provisional application No. 62/097,548, filed on Dec. 29, 2014.
(60) Provisional application No. 62/137,742, filed on Mar. 24, 2015.

(51) Int. Cl. A61K 9/00; A61K 47/26; A61K 47/28; A61K 31/4174; A61K 47/36; A61K 31/445; A61K 31/167; A61K 45/06; A61K 47/10; A61K 31/5375; A61K 31/47; A61K 31/46; A61K 31/4453; A61K 31/381; A61K 31/24; A61K 31/165; A61K 31/137; A61K 31/13; A61K 31/245; A61K 47/24; A61K 47/12; A61K 9/127; A61K 9/107; A61K 47/40

(52) U.S. Cl. CPC A61K 9/0043; A61K 47/40; A61K 47/28; A61K 31/4174; A61K 47/36; A61K 31/445; A61K 31/167; A61K 45/06; A61K 47/10; A61K 31/5375; A61K 31/47; A61K 31/46; A61K 31/4453; A61K 31/381; A61K 31/24; A61K 31/165; A61K 31/137; A61K 31/13; A61K 31/245; A61K 47/24; A61K 47/12; A61K 9/127; A61K 9/1075; A61K 47/26

(57) ABSTRACT

Provided herein are methods of locally and specifically anesthetizing nerve tissue in the trigeminal nerve pathway and methods of performing a surgical procedure on a subject in need thereof that include intranasally administering (1) a pharmaceutical composition that includes a local anesthetic agent and a mucosal absorption enhancer to a subject in need thereof, where the local anesthetic agent and mucosal absorption enhancer are present in an amount sufficient to anesthetize nerve tissue in the trigeminal nerve pathway upon administration, or (2) a pharmaceutical composition that include a free-base form of a local anesthetic agent, where the free-base form of the local anesthetic agent is present in an amount sufficient to anesthetize nerve tissue in the trigeminal nerve pathway upon administration.

### CROSS-REFERENCE TO RELATED APPLICATIONS

This application is a continuation of U.S. patent application Ser. No. 15/664,972, filed Jul. 31, 2017, which is a continuation of U.S. patent application Ser. No. 14/741,330, filed Jun. 16, 2015, which claims priority to U.S. Provisional Patent Application Ser. No. 62/012,946, filed Jun. 16, 2014; U.S. Patent Application Ser. No. 62/097,548, filed Dec. 29, 2014; and U.S. Provisional Patent Application Ser. No. 62/137,742, filed Mar. 24, 2015; the entire contents of each of these applications are hereby incorporated by reference.

### TECHNICAL FIELD

This invention relates to the field of anesthesia and drug delivery.

### BACKGROUND

The use of needles for anesthetizing nerve tissue in the trigeminal nerve pathway causes risk of exposure to blood-borne pathogens. The U.S. Federal Government has recognized the need to develop safer medical devices, including needle-free devices. U.S. government mandates require employers to implement safer medical devices that eliminate or minimize employee or customer exposure to blood-borne pathogens (Occupation Health and Safety Administration: Bloodborne Pathogens Standard, Needlestick Safety and Prevention Act).

Molar tooth removal is one of the common surgical procedures performed in the United Kingdom (Health Technol. Assess. 4:1-55, 2000). Surgical site infections are among the most common complications of molar tooth removal, with an estimated frequency of 1% to 30% (Susarla et al., Oral Maxillofac. Surg. Clin. North Am. 23:541-546, 2011). The injection of anesthetic agents into the tissue lining the mouth can be responsible for some of the infections associated with molar tooth removal.

### SUMMARY

Nasally administered local anesthetic agents were thought to undergo 100% transmucosal absorption when administered in the absence of any mucosal absorption enhancer. The present invention is based, at least in part, on the discovery that a mucosal absorption enhancer results in a further increase in the absorption (e.g., paracellular transport and/or transcellular transport) of local anesthetic agents across the nasal mucosa (e.g., a level of transport sufficient to anesthetize nerve tissue in the trigeminal nerve pathway, e.g., the mandibular nerve) and/or provides for a reduction in the administered dose (e.g., a safer and lower effective dose) of local anesthetic agent sufficient to anesthetize nerve tissue in the trigeminal nerve pathway (e.g., as compared to administration of the local anesthetic agent in the absence of the mucosal absorption enhancer). In view of this discovery, provided herein are methods of locally and specifically anesthetizing nerve tissue in the trigeminal nerve pathway and methods of performing a surgical procedure on a subject in need thereof that include intranasally administering a pharmaceutical composition that includes a local anesthetic agent and a mucosal absorption enhancer to a subject in need thereof, where the local anesthetic agent and mucosal absorption enhancer are present in an amount sufficient to anesthetize nerve tissue in the trigeminal nerve pathway upon administration. Also provided herein are methods of locally and specifically anesthetizing nerve tissue in the trigeminal nerve pathway and methods of performing a surgical procedure on a subject in need thereof that include intranasally administering a pharmaceutical composition that include a free-base form of a local anesthetic agent, where the free-base form of the local anesthetic agent is present in an amount sufficient to anesthetize nerve tissue in the trigeminal nerve pathway upon administration.

Provided herein are methods of locally and specifically anesthetizing nerve tissue in the trigeminal nerve pathway in a subject in need thereof that include intranasally administering a pharmaceutical composition including a local anesthetic agent and a mucosal absorption enhancer to a subject in need thereof, where: the local anesthetic agent and mucosal absorption enhancer are present in an amount sufficient to anesthetize nerve tissue in the trigeminal nerve pathway upon administration. Also provided are methods of performing a surgical procedure on a subject in need thereof that include: intranasally administering a pharmaceutical composition including a local anesthetic agent and a mucosal absorption enhancer to a subject in need thereof, wherein the local anesthetic agent and mucosal absorption enhancer are present in an amount sufficient to anesthetize nerve tissue in the trigeminal nerve pathway upon administration; and making at least one incision, puncture, or suture in at least one tissue selected from the group of: dental pulp, soft tissue in the oral cavity, eye, eye socket, eyelid, soft tissue of the face, and sinuses.

In some embodiments of these methods, at least 40% (e.g., at least 50% or at least 60%) of the local anesthetic agent is protonated at a pH of between about 4.0 and about 6.5. In some embodiments of these methods, at least 50% (e.g., at least 60%) of the local anesthetic agent is protonated at a pH of between about 5.0 and about 6.5.

In some embodiments of these methods, the mucosal absorption enhancer is selected from the group of: a monosaccharide, a polysaccharide (e.g., a linear polysaccharide, such as, e.g., chitosan, trimethyl chitosan, or a salt thereof), a bile salt (e.g., selected from the group of cholate, sodium taurocholate, sodium glycocholate, sodium deoxycholate, and sodium glycodeoxycholate), a surfactant (e.g., selected from the group of: sodium lauryl sulfate, polyoxyethyleneglycol dodecyl ether, polyoxyethylene 10 oleoyl ether, lysophosphatidylcholine, dioctyl sodium sulfosuccinate, polyoxyethylene 9 lauryl ether, polysorbate-80, polyethyleneglycol-8-laurate, and glyceryl monolaurate), an oil, a fusidate compound (e.g., sodium fusidate or sodium dihydrotaurofusidate), a cyclodextrin (e.g., selected from the group of: cyclodextrin, beta-cyclodextrin, hydroxypropyl beta-cyclodextrin, and dimethyl-beta-cyclodextrin), a phospholipid (e.g., selected from the group of: didecanoyl-L-mu-phosphatidylcholine, lysophosphatidylcholine, and didecanoyl-L-phosphatidylcholine), a thiomer (e.g., selected from the group of: chitosan-cysteine, chitosan-thiogylcolic acid, chitosan-thiobutylamidine, deacetylated gellan gum-cysteine, poly(methacrylic acid)-cysteine, alginate-cysteine, carboxymethycellulose-cysteine, poly(acrylic acid)-cysteine, poly(acrylic acid)-cysteamine, poly(acrylic acid)-homocysteine, and thiolated polycarbophil), a fatty acid (e.g., selected from the group of: monoglyceride, diglyceride, and triglyceride), a chelator (e.g., ethylene diamine tetraacetate or disodium ethylene diamine tetraacetate), a salicylate (e.g., selected from the group of: salicylic acid, sodium methoxysalicylate, and acetylsalicylic acid), a polymer (e.g., selected from the group of: polycarbonyl, sodium carboxymethylcellulose, and their derivatives), a micelle, an alcohol, a liposome, a microemulsion, an emulsion, a solid lipid nanoparticle, a transferosome, propylene glycol, menthol, ammonium glycyyhizinate, glycrrhetinic acid, aminated gelatin, laurocapram, benzalkonium chloride, a phenothiazine, a nitric acid donor, zonula occluden toxin, a poly-L-arginine, a soybean derivative glucoside, citicholine, and an alpha-acid derivative. In some examples of these methods, the monosaccharide is selected from the group of: glucose, galactose, mannose, 3-O-methyl glucose, xylose, ribose, arabinose, ribulose, fructose, and sorbose. In some examples of these methods, the salt of a chitosan or trimethyl chitosan is chitosan hydrochloride, chitosan glutamate, or trimethyl chitosan chloride. In some examples of these methods, the fatty acid is selected from the group of: sodium caprate, sodium lauryl sulfate, sorbitan laurate, sucrose palmitate, lauroyl choline, sodium myristate, palmitoyl carnitine, and their derivatives. In some examples of these methods, the fatty acid is a glyceride selected from the group of: a monohexanoin and a medium chain glyceride.

In some embodiments of these methods, the mucosal absorption enhancer is a liposome or a solid lipid nanoparticle. In some embodiments of these methods, the mucosal absorption enhancer is propylene glycol.

In some embodiments of these methods, the mucosal absorption enhancer is a microemulsion. In some embodiments of these methods, the microemulsion is an oil-in-water microemulsion including an aqueous phase, a lipid phase, and a surfactant. In some examples of these methods, the microemulsion further includes a co-surfactant. In some examples of these methods, the aqueous phase comprises at least about 10% by weight of the pharmaceutical composition. In some examples of these methods, the lipid phase comprises about 0.1% to about 50% by weight of the pharmaceutical composition. In some examples of these methods, one or both of the surfactant and the co-surfactant each includes about 0.1% to about 80% by weight of the pharmaceutical composition. In some embodiments of these methods, the lipid to total surfactant ratio in the pharmaceutical composition is between about 1:1 to about 1:10. In some embodiments of these methods, the surfactant to co-surfactant ratio in the pharmaceutical composition is between about 6:1 to about 1:6.

In some examples of these methods, the lipid is selected from the group of: castor oil, corn oil, cottonseed oil, olive oil, peanut oil, soybean oil, vegetable oil, coconut oil, isopropyl palmitate, light mineral oil, oleic acid, medium chain mono/diglycerides, and propylene glycol dicaprylocaprate. In some embodiments of these methods, the lipid is a local anesthetic agent in its oil form. In some embodiments of these methods, the surfactant and/or the co-surfactant is selected from the group of: polysorbate 20, polysorbate 40, polysorbate 60, polysorbate 65, polysorbate 80, polyoxyl 8 stearate, polypropylene glycol, propylene glycol, carbomer 1342, carbomer 934, carbomer 934P, carbomer 940, carbomer 941, carbomer 974, carbomer 980, carbomer 981, oleyl polyethylene glycol glyceride, PEG-300, PEG-400, PEG-3500, poloxamer 124, poloxamer 188, poloxamer 407, polyoxyl 35 castor oil, polyoxyl 40 hydrogenated castor oil, polyoxyl 40 stearate, polyoxyl 6 and polyoxyl 32 palmitostearate, polyoxyl 8 stearate, polyoxyl glyceryl stearate, polyoxyl lanolin, polyoxyl palmitate, polyoxyl stearate, glyceryl oleate, propylene carbonate, sorbitan monooleate, glycerin, PEG-5 oleate, sodium N-methyl N-cocoyl taurate, PEG-8 caprylic/capric gylcerides, and diethylene glycol monoethyl ether.

In some embodiments of these methods, the mucosal absorption enhancer comprises about 0.001% to about 50% (e.g., about 0.1% to about 25%, about 0.1% to about 5.0%, or about 1.0% to about 5.0%) by weight of the composition.

In some examples of these methods, the composition further includes a vasoconstrictor (e.g., selected from the group of: oxymetazoline, phenylephrine, naphazoline, propylhexadrine, levodesoxyephedrine, epinephrine, norepinephrine, and a salt thereof). In some embodiments of these methods, the vasoconstrictor includes about 0.001% to about 10% (e.g., about 0.001% to about 5%, or about 0.001% to about 1%) by weight of the composition.

In some embodiments of these methods, all of the mandibular nerve is anesthetized. In some embodiments of these methods, the subject is in need of oral surgery (e.g., extraction or a tooth, such as, e.g., a molar tooth) or a dental procedure.

Some embodiments of these methdos further include performing oral surgery or a dental procedure on the subject. In some embodiments of these methods, the subject is in need of ocular or eyelid surgery or an ocular or eyelid procedure. Some embodiments further include performing the ocular or eyelid procedure on the subject or performing ocular or eyelid surgery on the subject.

In some embodiments of these methods, the subject is need of exterior nose surgery, sinus surgery, or a sinus procedure. Some embodiments of these methods further include performing exterior nose surgery on the subject, performing sinus surgery on the subject, or performing the sinus procedure on the subject.

In some embodiments of these methods, the subject is in need of oral surgery (e.g., extraction of a tooth, such as, e.g., a molar tooth) and the nerve tissue is one or more of anterior superior alveolar nerve, middle superior alveolar nerve, infraorbital nerve entering the infraorbtial canal, posterior superior alveolar nerve, maxillary nerve, mandibular nerve, and trigeminal nerve.

In some embodiments of these methods, the subject is in need of ocular or eyelid surgery. In some embodiments of these methods, the subject is need of exterior nose surgery or sinus surgery.

Also provided are methods of locally and specifically anesthetizing nerve tissue in the trigeminal nerve pathway in a subject in need thereof that include: intranasally administering a pharmaceutical composition including a free-base form of a local anesthetic agent to a subject in need thereof, where: the free-base form of the local anesthetic agent is present in an amount sufficient to anesthetize nerve tissue in the trigeminal nerve pathway upon administration. Also provided are methods of performing a surgical procedure on a subject in need thereof that include: intranasally administering a pharmaceutical composition including a free-base form of a local anesthetic agent to a subject in need thereof, where the free-base form of the local anesthetic agent is present in an amount sufficient to anesthetize nerve tissue in the trigeminal nerve pathway upon administration; and making at least one incision, puncture, or suture in at least one tissue selected from the group of: dental pulp, soft tissue in the oral cavity, eye, eye socket, eyelid, soft tissue of the face, and sinuses.

In some embodiments of these methods, the pharmaceutical composition further includes a drug delivery vehicle (e.g., selected from the group of a liposome, a microemulsion, an emulsion, a micelle, a lipid nanoparticle, and a cyclodextrin). In some embodiments of these methods, the drug delivery vehicle encapsulates or entraps the local anesthetic agent. In some embodiments of these methods, the liposome is selected from the group of: a small unilamellar vesicle, a large unilamellar vesicle, a giant unilamellar vesicle, a multilamellar vesicle, and a multivesicular vesicle. In some embodiments of these methods, the liposome in the pharmaceutical composition has a net charge that is cationic, neutral, or anionic. In some examples of these methods, the liposome includes a phospholipid. In some examples of these methods, the phospholipid is selected from the group of: a phosphatidylcholine, a lysophosphatidylcholine, a phosphatidylserine, a phosphatidylethanolamine, a phosphatidylglycerol, and a phosphatidylinositol. In some embodiments of these methods, the liposome further includes an excipient (e.g., selected from the group of: a cholesterol, a stearylamine, a stearic acid, a tocopherol, a polymer, a chitosan, and a polyacrylic acid).

In some embodiments of these methods, the microemulsion includes an oil, water, and a surfactant. In some embodiments of these methods, the microemulsion is selected from the group of: an oil-in-water microemulsion, a water-in-oil microemulsion, and a bi-continuous microemulsion. In some embodiments of these methods, the cyclodextrin is selected from the group of: cyclodextrin, beta-cyclodextrin, hydroxypropyl beta-cyclodextrin, and dimethyl-beta-cyclodextrin.

In some embodiments of these methods, the drug delivery vehicle comprises about 0.001% to about 50% (e.g., about 0.1% to about 25%, about 1.0% to about 18.0%, or about 1.0% to about 10%) by weight of the composition.

In some embodiments of these methods, the composition further includes a mucosal absorption enhancer (e.g., selected from the group of a bile salt, a surfactant, a fatty acid, a chelator, a salicylate compound, a polymer, a monosaccharide, a polysaccharide, a chitosan, a thiomer, menthol, ammonium glycyyhizinate, glycrrhetinic acid, aminated gelatin, laurocapram, benzalkonium chloride, a phenothiazine, a nitric acid donor, zonula occluden toxin, a poly-L-arginine, a soybean derivative glucoside, citicholine, and an alpha-acid derivative). In some examples of these methods, the mucosal absorption enhancer includes about 0.001% to about 50% by weight of the pharmaceutical composition.

In some embodiments of these methods, the subject is in need of oral surgery (e.g., extraction of a tooth, such as, e.g., a molar tooth) or a dental procedure. In some examples of these methods, all of the mandibular nerve is anesthetized. Some embodiments further include performing oral surgery or a dental procedure on the subject.

In some embodiments of these methods, the subject is in need of ocular or eyelid surgery or an ocular or eyelid procedure. Some embodiments of these methods further include performing the ocular or eyelid procedure on the subject, or performing ocular or eyelid surgery on the subject.

In some embodiments of these methods, the subject is need of exterior nose surgery, sinus surgery, or a sinus procedure. Some embodiments of these methods further include performing exterior nose surgery on the subject, performing sinus surgery on the subject, or performing the sinus procedure on the subject. In some examples of these methods, the subject is in need of oral surgery (e.g., extraction of a tooth, such as, e.g., a molar tooth) and the nerve tissue is one more of anterior superior alveolar nerve, middle superior alveolar nerve, infraorbital nerve entering the infraorbtial canal, posterior superior alveolar nerve, maxillary nerve, mandibular nerve, and trigeminal nerve. In some examples of these methods, all of the mandibular nerve is anesthetized.

In some examples of these methods, the subject is in need of ocular or eyelid surgery. In some embodiments of these methods, the subject is need of exterior nose surgery or sinus surgery.

In some examples of any of the methods described herein, the nerve tissue in the trigeminal nerve pathway includes one or more of trigeminal ganglion, the frontal nerve, the supraorbital nerve, the supratrochlear nerve, the lacrimal nerve, the nasociliary nerve, the infratrochlear nerve, the ciliary nerve, the anterior ethmoidal nerve, the external nasal nerve, the internal nasal nerve, the ophthalmic nerve (V1), the infraorbital nerve, the anterior superior alveolar nerve, the middle superior alveolar nerve, the infraorbital nerve entering the infraorbital canal, the posterior superior alveolar nerve, the ganglionic branches to pterygopalatine ganglion, the pterygopalatine ganglion, the greater and lesser palantine nerves, the lateral nasal branches of the greater palatine nerve, nerve of the pterygoid canal, the nasopalatine nerve, the zygomatic nerve, the zygomaticofacial nerve, the zygomaticotemporal nerve, the lateral nasal branches of the maxillary nerve, the maxillary nerve (V2), the buccal nerve, the auriculotemporal nerve, the lingual nerve, the inferior alveolar nerve, the mylohyoid nerve, the mental nerve, the incisive nerve, and the mandibular nerve (V3). In some embodiments of any of the methods described herein, the nerve tissue includes the mandibular nerve (V3).

In some embodiments of any of the methods described herein, the pharmaceutical composition is directly administered onto one or more of the following nasal tissues: the inferior nasal turbinate, the middle nasal turbinate, the nasal meatuses, the extreme rear of the nasal cavity, and the maxillary sinus. In some embodiments of any of the methods described herein, the pharmaceutical composition is directly placed on one or more of the inferior nasal turbinate, the middle nasal turbinate, the nasal meatuses, the extreme rear of the nasal cavity, and the maxillary sinus.

In some embodiments of any of the methods described herein, the local anesthetic agent is selected from the group of: benzocaine, butacaine, tetracaine, lidocaine, dyclonine, pramoxine, dibucaine, cocaine, etidocaine, bupivacaine, levobupivacaine, ropivacaine, procaine, chloroprocaine, mepivacaine, prilocaine, articaine, hexylcaine, oxetacaine, and a salt thereof. In some embodiments of any of the methods described herein, the local anesthetic agent comprises about 0.1% to about 50% by weight of the composition.

In some embodiments of any of the methods described herein, the composition further includes a preservative and/or a viscosity enhancing agent. In some embodiments of any of the methods described herein, the preservative is selected from the group of: sorbitol, mannitol, ethanol, benzyl alcohol, isopropanol, cresol, chlorocresol, phenol, and benzalkonium chloride. In some examples of any of the methods described herein, the preservative comprises about 0.1% to about 5% weight of the composition. In some embodiments of any of the methods described herein, the viscosity enhancing agent is selected from the group of: hydroxyethylcellulose, hydroxypropylmethyl cellulose, sodium carboxy methyl cellulose, carbomer homopolymer type a, carbomer homopolymer type b, carbomer, polycarbophil, sodium alginate, xantham gum, smart hydrogel, polyethylene glycol, hydroxycellulose, poloxamer 188, poloxamer 407, starch, aminated gelatin, chitosan, hydroxypropyl methylcellulose, hydroxypropyl cellulose, methylcellulose, carboxymethyl cellulose, ethylcellulose, and microcrystalline cellulose. In some embodiments of any of the methods described herein, the viscosity enhancing agent comprises about 0.01% to about 10% by weight of the pharmaceutical composition.

In some examples of any of the methods described herein, the composition further includes a pharmaceutically acceptable carrier. In some embodiments of any of the methods described herein, the composition further includes an antihistamine. In some examples of any of the methods described herein, the antihistamine is selected from the group of: atropine, azelastine, hydroxyzine, desloratadine, cyproheptadine, emadastine, levocabastine, azelastine, carbinoxamine, levocetirizine, fexofenadine, diphenhydramine, brompheniramine, loratadine, clemastine, chlorpheniramine, certirizine, and a salt thereof.

Also provided are pharmaceutical compositions that include a local anesthetic agent and a mucosal absorption enhancer, where the composition is formulated for intranasal administration and the local anesthetic agent and mucosal absorption enhancer are present in an amount sufficient to anesthetize nerve tissue in the trigeminal nerve pathway upon nasal administration. In some embodiments of these pharmaceutical compositions, the local anesthetic agent is selected from the group of: benzocaine, butacaine, tetracaine, lidocaine, dyclonine, pramoxine, dibucaine, cocaine, etidocaine, bupivacaine, levobupivacaine, ropivacaine, procaine, chloroprocaine, mepivacaine, prilocaine, articaine, hexylcaine, oxetacaine, and a salt thereof. In some examples of these pharmaceutical compositions, at least 40% (e.g., at least 50% or at least 60%) of the local anesthetic agent is protonated at a pH of between about 4.0 and about 6.5. In some examples of these pharmaceutical compositions, at least 50% (e.g., a tleast 60%) of the local anesthetic agent is protonated at a pH of between about 5.0 and about 6.5. In some examples of these pharmaceutical compositions, the local anesthetic agent comprises about 0.1% to about 50% by weight of the composition.

In some examples of the pharmaceutical compositions, the mucosal absorption enhancer is selected from the group of: a monosaccharide (e.g., selected from the group of: glucose, galactose, mannose, 3-O-methyl glucose, xylose, ribose, arabinose, ribulose, fructose, and sorbose), a polysaccharide (e.g., a linear polysaccharide, such as, e.g., chitosan, trimethyl chitosan, or a salt thereof), a bile salt (e.g., selected from the group of: cholate, sodium taurocholate, sodium glycocholate, sodium deoxycholate, and sodium glycodeoxycholate), a surfactant, an oil, a fusidate compound (e.g., selected from the group of: sodium fusidate and sodium dihydrotaurofusidate), a cyclodextrin (e.g., selected from the group of: cyclodextrin, beta-cyclodextrin, hydroxypropyl beta-cyclodextrin, and dimethyl-beta-cyclodextrin), a phospholipid (e.g., selected from the group of: didecanoyl-L-mu-phosphatidylcholine, lysophosphatidylcholine, and didecanoyl-L-phosphatidylcholine), a thiomer (e.g., selected from the group of: chitosan-cysteine, chitosan-thiogylcolic acid, chitosan-thiobutylamidine, deacetylated gellan gum-cysteine, poly(methacrylic acid)-cysteine, alginate-cysteine, carboxymethycellulose-cysteine, poly(acrylic acid)-cysteine, poly(acrylic acid)-cysteamine, poly(acrylic acid)-homocysteine, and thiolated polycarbophil), a fatty acid (e.g., selected from the group of: monoglyceride, diglyceride, and triglyceride.), a chelator (e.g., ethylene diamine tetraacetate or disodium ethylene diamine tetraacetate), a salicylate (e.g., selected from the group of: salicylic acid, sodium methoxysalicylate, and acetylsalicylic acid), a polymer (e.g., selected from the group of: polycarbonyl, sodium carboxymethylcellulose, and their derivatives), a micelle, an alcohol, a liposome, a microemulsion, an emulsion, a solid lipid nanoparticle, a transferosome, propylene glycol, menthol, ammonium glycyyhizinate, glycrrhetinic acid, aminated gelatin, laurocapram, benzalkonium chloride, a phenothiazine, a nitric acid donor, zonula occluden toxin, a poly-L-arginine, a soybean derivative glucoside, citicholine, and an alpha-acid derivative. In some examples of these pharmaceutical compositions, the salt of chitosan or trimethyl chitosan is chitosan hydrochloride, chitosan glutamate, or trimethyl chitosan chloride.

In some examples of these pharmaceutical compositions, the fatty acid is selected from the group of: sodium caprate, sodium lauryl sulfate, sorbitan laurate, sucrose palmitate, lauroyl choline, sodium myristate, palmitoyl carnitine, and their derivatives. In some examples of these methods, the fatty acid is a glyceride selected from the group of: a monohexanoin and a medium chain glyceride. In some examples of any of the methods described herein, the surfactant is selected from the group of: sodium lauryl sulfate, polyoxyethyleneglycol dodecyl ether, polyoxyethylene 10 oleoyl ether, lysophosphatidylcholine, dioctyl sodium sulfosuccinate, polyoxyethylene 9 lauryl ether, polysorbate-80, polyethyleneglycol-8-laurate, and glyceryl monolaurate.

In some examples of these pharmaceutical compositions, the mucosal absorption enhancer is a liposome or a solid lipid nanoparticle. In some examples of these pharmaceutical compositions, the mucosal absorption enhancer is propylene glycol.

In some examples of these pharmaceutical compositions, the mucosal absorption enhancer is a microemulsion. In some embodiments of these pharmaceutical compositions, the microemulsion is an oil-in-water microemulsion that includes an aqueous phase, a lipid phase, and a surfactant. In some examples of these pharmaceutical compositions, the microemulsion further includes a co-surfactant. In some examples of these pharmaceutical compositions, the aqueous phase includes at least about 10% by weight of the pharmaceutical composition. In some examples of these pharmaceutical compositions, the lipid phase comprises about 0.1% to about 50% by weight of the pharmaceutical composition. In some examples of these pharmaceutical compositions, one or both of the surfactant and the co-surfactant each comprises about 0.1% to about 80% by weight of the pharmaceutical composition. In some embodiments of these pharmaceutical compositions, the lipid to total surfactant ratio in the pharmaceutical composition is between about 1:1 to about 1:10. In some embodiments of these pharmaceutical compositions, the surfactant to co-surfactant ratio in the pharmaceutical composition is between about 6:1 to about 1:6. In some embodiments of these pharmaceutical compositions, the lipid is selected from the group of: castor oil, corn oil, cottonseed oil, olive oil, peanut oil, soybean oil, vegetable oil, coconut oil, isopropyl palmitate, light mineral oil, oleic acid, medium chain mono/diglycerides, and propylene glycol dicaprylocaprate. In some embodiments of these pharmaceutical compositions, the lipid is a local anesthetic agent in its oil form. In some examples of these pharmaceutical compositions, the surfactant and/or the co-surfactant is selected from the group of: polysorbate 20, polysorbate 40, polysorbate 60, polysorbate 65, polysorbate 80, polyoxyl 8 stearate, polypropylene glycol, propylene glycol, carbomer 1342, carbomer 934, carbomer 934P, carbomer 940, carbomer 941, carbomer 974, carbomer 980, carbomer 981, oleyl polyethylene glycol glyceride, PEG-300, PEG-400, PEG-3500, poloxamer 124, poloxamer 188, poloxamer 407, polyoxyl 35 castor oil, polyoxyl 40 hydrogenated castor oil, polyoxyl 40 stearate, polyoxyl 6 and polyoxyl 32 palmitostearate, polyoxyl 8 stearate, polyoxyl glyceryl stearate, polyoxyl lanolin, polyoxyl palmitate, polyoxyl stearate, glyceryl oleate, propylene carbonate, sorbitan monooleate, glycerin, PEG-5 oleate, sodium N-methyl N-cocoyl taurate, PEG-8 caprylic/capric gylcerides, and diethylene glycol monoethyl ether. In some examples of these pharmaceutical compositions, the mucosal absorption enhancer comprises about 0.001% to about 50% (e.g., about 0.1% to about 25%, about 0.1% to about 5.0%, or about 1.0% to about 5.0%) by weight of the composition.

Some examples of these pharmaceutical compositions further include a vasoconstrictor (e.g., selected from the group of: oxymetazoline, phenylephrine, naphazoline, propylhexadrine, levodesoxyephedrine, epinephrine, norepinephrine, and a salt thereof). In some examples of these pharmaceutical compositions, the vasoconstrictor comprises about 0.001% to about 10% (e.g., about 0.001% to about 5%, or about 0.001% to about 1%) by weight of the composition.

Some examples of these pharmaceutical compositions further include a preservative and/or a viscosity enhancing agent. In some examples of these pharmaceutical compositions, the preservative is selected from the group of: sorbitol, mannitol, ethanol, benzyl alcohol, isopropanol, cresol, chlorocresol, phenol, and benzalkonium chloride. In some embodiments of these pharmaceutical compositions, the preservative comprises about 0.1% to about 5% weight of the composition. In some examples of these pharmaceutical compositions, the viscosity enhancing agent is selected from the group of: hydroxyethylcellulose, hydroxypropylmethyl cellulose, sodium carboxy methyl cellulose, carbomer homopolymer type a, carbomer homopolymer type b, carbomer, polycarbophil, sodium alginate, xantham gum, smart hydrogel, polyethylene glycol, hydroxycellulose, poloxamer 188, poloxamer 407, starch, aminated gelatin, chitosan, hydroxypropyl methylcellulose, hydroxypropyl cellulose, methylcellulose, carboxymethyl cellulose, ethylcellulose, and microcrystalline cellulose. In some examples of these pharmaceutical compositions, the viscosity enhancing agent comprises about 0.01% to about 10% by weight of the pharmaceutical composition.

Some embodiments of these pharmaceutical compositions further include a pharmaceutically acceptable carrier. Some embodiments of these pharmaceutical compositions further include an antihistamine (e.g., selecting from the group of atropine, azelastine, hydroxyzine, desloratadine, cyproheptadine, emadastine, levocabastine, azelastine, carbinoxamine, levocetirizine, fexofenadine, diphenhydramine, brompheniramine, loratadine, clemastine, chlorpheniramine, certirizine, and a salt thereof).

Also provided are pharmaceutical compositions that include a free-base form of a local anesthetic agent, wherein the composition is formulated for intranasal administration and the free-base form of the local anesthetic agent is present in an amount sufficient to anesthetize nerve tissue in the trigeminal nerve pathway upon nasal administration. In some embodiments of these pharmaceutical compositions, the local anesthetic agent is selected from the group of: benzocaine, butacaine, tetracaine, lidocaine, dyclonine, pramoxine, dibucaine, cocaine, etidocaine, bupivacaine, levobupivacaine, ropivacaine, procaine, chloroprocaine, mepivacaine, prilocaine, articaine, hexylcaine, and oxetacaine. In some embodiments of these pharmaceutical compositions, the local anesthetic agent comprises about 0.1% to about 50% by weight of the composition.

Some embodiments of these pharmaceutical compositions further include a drug delivery vehicle (e.g., selected from the group of: a liposome, a microemulsion, an emulsion, a micelle, a lipid nanoparticle, and a cyclodextrin). In some examples of these pharmaceutical compositions, the drug delivery vehicle encapsulates or entraps the local anesthetic agent. In some examples of these pharmaceutical agents, the liposome is selected from the group of: a small unilamellar vesicle, a large unilamellar vesicle, a giant unilamellar vesicle, a multilamellar vesicle, and a multivesicular vesicle. In some examples of these pharmaceutical compositions, the liposome in the composition has a net charge that is cationic, neutral, or anionic. In some embodiments of these pharmaceutical compositions, the liposome includes a phospholipid (e.g., selected from the group of: a phosphatidylcholine, a lysophosphatidylcholine, a phosphatidylserine, a phosphatidylethanolamine, a phosphatidylglycerol, and a phosphatidylinositol). In some embodiments of these pharmaceutical compositions, the liposome further includes an excipient (e.g., selected from the group of: a cholesterol, a stearylamine, a stearic acid, a tocopherol, a polymer, a chitosan, and a polyacrylic acid).

In some examples of these pharmaceutical compositions, the microemulsion includes an oil, water, and a surfactant. In some examples of the pharmaceutical compositions, the microemulsion is selected from the group of: an oil-in-water microemulsion, a water-in-oil microemulsion, and a bi-continuous microemulsion. In some embodiments of these pharmaceutical compositions, the cyclodextrin is selected from the group of: cyclodextrin, beta-cyclodextrin, hydroxypropyl beta-cyclodextrin, and dimethyl-beta-cyclodextrin.

In some embodiments of these pharmaceutical compositions, the drug delivery vehicle includes about 0.001% to about 50% (e.g., about 0.1% to about 25%, about 1.0% to about 18.0%, or about 1.0% to about 10.0%) by weight of the composition. Some examples of these pharmaceutical compositions further include one or both of a preservative and a viscosity enhancing agent. In some examples of these pharmaceutical compositions, the preservative is selected from the group of: sorbitol, mannitol, ethanol, benzyl alcohol, isopropanol, cresol, chlorocresol, phenol, and benzalkonium chloride. In some examples of these pharmaceutical compositions, thepreservative comprises about 0.1% to about 5% weight of the composition. In some examples of these pharmaceutical compositions, the viscosity enhancing agent is selected from the group of: hydroxyethylcellulose, hydroxypropylmethyl cellulose, sodium carboxy methyl cellulose, carbomer homopolymer type a, carbomer homopolymer type b, carbomer, polycarbophil, sodium alginate, xantham gum, smart hydrogel, polyethylene glycol, hydroxycellulose, poloxamer 188, poloxamer 407, starch, aminated gelatin, chitosan, hydroxypropyl methylcellulose, hydroxypropyl cellulose, methylcellulose, carboxymethyl cellulose, ethylcellulose, and microcrystalline cellulose. In some examples of these pharmaceutical compositions, the viscosity enhancing agent comprises about 0.01% to about 10% by weight of the pharmaceutical composition.

Some embodiments of these pharmaceutical compositions further include a pharmaceutically acceptable carrier. Some embodiments of these pharmaceutical compositions further include an antihistamine. In some examples of these pharmaceutical compositions, the antihistamine is selected from the group of: atropine, azelastine, hydroxyzine, desloratadine, cyproheptadine, emadastine, levocabastine, azelastine, carbinoxamine, levocetirizine, fexofenadine, diphenhydramine, brompheniramine, loratadine, clemastine, chlorpheniramine, certirizine, and a salt thereof.

Some embodiments of these pharmaceutical compositions further include a mucosal absorption enhancer (e.g., selected from the group of: a bile salt, a surfactant, a fatty acid, a chelator, a salicylate compound, a polymer, a monosaccharide, a polysaccharide, a chitosan, a thiomer, menthol, ammonium glycyyhizinate, glycrrhetinic acid, aminated gelatin, laurocapram, benzalkonium chloride, a phenothiazine, a nitric acid donor, zonula occluden toxin, a poly-L-arginine, a soybean derivative glucoside, citicholine, and an alpha-acid derivative). In some examples of these pharmaceutical compositions, the mucosal absorption enhancer comprises about 0.1% to about 10% by weight of the composition.

Also provided are kits including any of the pharmaceutical compositions described herein.

As used herein, the word “a” before a noun represents one or more of the particular noun. For example, the phrase “a local anesthetic agent” represents “one or more local anesthetic agents.”

The term “subject” means a vertebrate, including any member of the class mammalia, including humans, sports or pet animals, such as horse (e.g., race horse) or dog (e.g., race dogs), and higher primates. In preferred embodiments, the subject is a human.

The term “anesthetizing” means blocking or significantly reducing (e.g., by at least 60%, by at least 70%, by at least 80%, by at least 90%, by at least 95%, or by at least 98%) the electrochemical activation of nerve tissue (e.g., blocking the action potential of nerves in a nerve tissue in the trigeminal nerve pathway) and/or blocking the propagation of electrochemical impulses through a nerve tissue (e.g., blocking propagation of action potential through a nerve tissue in the trigeminal nerve pathway). The anesthetization of nerve tissue (e.g., nerve tissue in the trigeminal nerve pathway) can include the blocking of pain signals.

The term “drug delivery vehicle” is art known and means a carrier system (e.g., a liposome, a microemulsion, an emulsion, a micelle, and a lipid nanoparticle) capable of controlling the release, solubility, absorption, and/or biodistribution of a pharmaceutical agent (e.g., a local anesthetic agent). A drug delivery vehicle can, e.g., encapsulate or entrap a local anesthetic agent in its free-base form in any of the pharmaceutical compositions described herein.

The term “liposome” is art known and means vesicular lipid bilayer enclosing an aqueous region. Liposomes can have, e.g., an average diameter of between about 2.5 nm to about 3000 nm. Liposomes can include one or more of the following: a phospholipid, an excipient, and a local anesthetic agent (e.g., a local anesthetic agent in its free-base form). For example, a liposome can encapsulate a local anesthetic agent (e.g., a local anesthetic agent in its free-base form) within the aqueous region of the liposome and/or can entrap the local anesthetic agent (e.g., a local anesthetic agent in its free-base form) within the lipid bilayer of the liposome. Non-limiting examples of liposomes include: small unilamellar vesicles, large unilamellar vesicles, giant unilamellar vesicles, a multilamellar vesicles, and a multivesicular vesicles. Additional exemplary aspects of liposomes are described herein.

The term “microemulsion” is art known and generally means a system of water, oil, a surfactant, and optionally a co-surfactant. A microemulsion is a thermodynamically stable, colloidal mixture. A microemulsion can be, e.g., classified by its continuous phase and dispersed phase. For example, a microemulsion can be oil-in-water (o/w), water-in-oil (w/o), or bi-continuous. A microemulsion can include droplets having an average diameter of between about 1 nm to about 100 nm (e.g., between about 10 nm to about 50 nm). A local anesthetic agent (e.g., a local anesthetic agent in its free-base form) can be entrapped by one or more droplets present in a microemulsion. Non-limiting aspects of microemulsions are described herein.

The term “emulsion” is art known and means a dispersion of droplets of one liquid in another liquid in which it is normally immiscible. An emulsion is a colloid. An emulsion can entrap a local anesthetic agent (e.g., a local anesthetic agent in its free-base form) in one or more of its droplets. The droplets in an emulsion can have an average diameter of between about 10 nm to about 100 μm. Non-limiting aspects of emulsions are described herein.

The term “micelle” is art known and generally means a lipid monolayer enclosing a hydrophobic region. A micelle can encapsulate a local anesthetic agent (e.g., a local anesthetic agent in its free-base form) in its enclosed hydrophobic region and/or can entrap a local anesthetic agent (e.g., a local anesthetic agent in its free-base form) in its lipid monolayer. Micelles can include amphiphilic molecules, e.g., amphiphilic di- or tri-block copolymers. Non-limiting aspects of micelles are described herein.

The term “lipid nanoparticle” or “solid lipid nanoparticle” is art known and means a particle that includes a solid lipid core matrix that is stabilized by a surfactant. A lipid nanoparticle can entrap or encapsulate a local anesthetic agent (e.g., a local anesthetic agent in its free-base form) within its solid lipid core matrix. A lipid nanoparticle can have an average diameter of between about 10 nm to about 1000 nm. Additional non-limiting aspects of lipid nanoparticles are described herein.

The term “local anesthetic agent” is art known and means an agent capable of penetrating a tissue (e.g., a nasal mucosa) deep enough (e.g., to a depth of greater than 4 mm, greater than 5 mm, greater than 6 mm, or greater than 10 mm) so as to reach the targeted nerve tissue (e.g., nerve branches of the targeted nerve tissue) and to reversibly (e.g., within 8 hours, within 6 hours, within 5 hours, within 4 hours, within 3 hours, or within 2 hours) anesthetize the targeted nerve tissue (e.g., nerve tissue in the trigeminal nerve pathway). A local anesthetic agent can, e.g., have a desensitizing effect on tissue innervated by the targeted nerve tissue.

The term “free-base form of a local anesthetic agent,” “local anesthetic agent free-base,” or “local anesthetic agent in its free-base form” is art known and means an uncharged form of local anesthetic agent. Non-limiting examples of the free-base form of several local anesthetic agents are described herein. Any of the compositions or pharmaceutical compositions described herein can include a detectable or significant amount of a free-base form of a local anesthetic agent.

The phrase “local and specific anesthetizing” is art known and means the reversible (e.g., within 8 hours, within 6 hours, within 5 hours, within 4 hours, within 3 hours, or within 2 hours) anesthetization of a targeted nerve tissue (e.g., nerve tissue in the trigeminal nerve pathway) that includes the administration of a composition including a local anesthetic agent, where the local anesthetic agent penetrates a tissue in a subject deep enough (e.g., to a depth of greater than 4 mm, greater than 5 mm, greater than 6 mm, or greater than 10 mm) and the local anesthetic agent does not mediate substantial anesthetization of non-targeted nerve tissue. In some examples, local and specific anesthetizing provides a desensitizing effect in tissues innervated by the targeted nerve tissue.

The term “trigeminal nerve pathway” or “trigeminal nerve” is art known and means the fifth cranial nerve and all of its branches. The trigeminal nerve pathway includes, e.g., trigeminal ganglion, the frontal nerve, the supraorbital nerve, the supratrochlear nerve, the lacrimal nerve, the nasociliary nerve, the infratrochlear nerve, the ciliary nerve, the anterior ethmoidal nerve, the external nasal nerve, the internal nasal nerve, the ophthalmic nerve (V1), the infraorbital nerve, the anterior superior alveolar nerve, the middle superior alveolar nerve, the infraorbital nerve entering the infraorbital canal, the posterior superior alveolar nerve, the ganglionic branches to pterygopalatine ganglion, the pterygopalatine ganglion, the greater and lesser palantine nerves, the lateral nasal branches of the greater palatine nerve, nerve of the pterygoid canal, the nasopalatine nerve, the zygomatic nerve, the zygomaticofacial nerve, the zygomaticotemporal nerve, the lateral nasal branches of the maxillary nerve, the maxillary nerve (V2), the buccal nerve, the auriculotemporal nerve, the lingual nerve, the inferior alveolar nerve, the mylohyoid nerve, the mental nerve, the incisive nerve, and the mandibular nerve (V3).

The term “mucosal absorption enhancer” is art known and means an agent that increases the residence time of a pharmaceutical composition placed onto nasal mucosa and/or increases the paracellular transport of a local anesthetic agent (e.g., any of the local anesthetic agents described herein). Non-limiting examples of mucosal absorption enhancers are described herein. Additional examples of mucosal absorption enhancers are known in the art. Exemplary assays for testing the ability of a mucosal absorption enhancer to increase the residence time of a pharmaceutical composition placed onto nasal mucosa and assays for measuring the ability of a mucosal absorption enhancer to increase paracellular transport of a local anesthetic agent (e.g., any of the local anesthetic agents described herein) are described herein. Additional assays for testing the ability of a mucosal absorption enhancer to increase residence time of a pharmaceutical composition placed onto nasal mucosa and assays for measuring the ability of a mucosal absorption enhancer are known in the art.

The term “preservative” is art known and means an agent that decreases the rate of growth of bacteria (e.g., mycobacteria), fungi, and/or viruses when present in a composition.

The term “viscosity enhancing agent” is art known and means an agent that is typically added to a nasal pharmaceutical composition that enhances the thickness and reduces the flow rate of the nasal pharmaceutical composition (e.g., both before and after administration to a subject).

The term “antihistamine” is art known and means an agent that inhibits the action of histamine by blocking its attachment to histamine receptors (e.g., in a human body) and/or inhibiting the enzymatic activity of histidine decarboxylase (which catalyzes the transformation of histidine into histamine). Non-limiting examples of antihistamines are described herein. Additional examples of antihistamines are known in the art.

The term “surgery” is art known and means a medical procedure that includes at least one of an incision, debridement, placement of a suture, or a puncture into a tissue present in a subject's body (e.g., a human body).

The term “oral surgery” is art known and means a medical procedure that includes at least one of an incision, debridement, placement of a suture, or a puncture into the soft tissue (e.g., oral gingiva, cheeks, lips, and/or tongue) (or debridement of a soft tissue) within a subject's mouth (e.g., a human's mouth).

The term “sinus surgery” is art known and means a medical procedure that includes at least one of an incision, a debridement, placement of a suture, or a puncture in a subject's sinus tissue.

The term “ocular surgery” is art known and means a medical procedure that includes at least one of an incision, a debridement, placement of a suture, or a puncture in one of more of the eye (e.g., any part of the eye), eye socket, and the glandular tissue surrounding the eye of a subject (e.g., a human subject).

The term “eyelid surgery” is art known and means a medical procedure that includes at least one incision, a debridement, placement of a suture, or a puncture in one or more of the eyelid (e.g., any part of an eyelid) or the glandular tissue surrounding the eyelid of a subject (e.g., a human subject).

The term “exterior nose surgery” means a medical procedure that includes at least one incision, a debridement (e.g., laser debridement), placement of a suture, and a puncture in the outer skin and/or cartilage of a subject's nose (e.g., a human's nose).

The term “dental procedure” means a treatment by a dental professional that includes one or more of manipulating, repositioning, or debriding one or more teeth or the material on one or more teeth in a subject (e.g., a human) and/or one or more of manipulating or repositioning the gums and/or soft tissue (e.g., gums) in a subject (e.g., a human subject).

The term “ocular procedure” means a treatment by a medical professional that includes one or more of manipulating and repositioning an eye (e.g., any part of an eye), an eye socket, and a glandular tissue surrounding an eye in a subject (e.g., a human subject).

The term “eyelid procedure” means a treatment by a medical professional that includes manipulating an eyelid (e.g., any part of an eyelid) or a glandular tissue surrounding an eyelid in a subject (e.g., a human subject).

The term “sinus procedure” means a treatment by a medical professional that includes one or more of manipulating a sinus tissue, restructuring a sinus tissue, and implanting a medical device within a sinus tissue (e.g., via an endoscopic procedure) in a subject (e.g., a human subject).

The terms “nasal mucosa” or “nasal mucus membrane” are art known and mean tissue that lines the nasal cavity and maxillary sinus.

The term “mandibular dental arch” or “mandibular arch” is art known and refers to the curved structure formed by the mandibular (lower) teeth in their natural position.

The term “maxillary dental arch” or “maxillary arch” is art known and refers to the curved structure formed by the maxillary teeth in their natural position.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 schematic diagram showing non-limiting exemplary nerve tissues in the trigeminal nerve pathway.

FIG. 2 is a schematic diagram showing non-limiting exemplary nerve tissues in the trigeminal nerve pathway.

FIG. 3 is a schematic diagram showing non-limiting exemplary nasal tissue for the administration of any pharmaceutical composition provided herein.

### DETAILED DESCRIPTION

Provided herein are methods of locally and specifically anesthetizing nerve tissue in the trigeminal nerve pathway and methods of performing a surgical procedure on a subject in need thereof that include intranasally administering a pharmaceutical composition that includes a local anesthetic agent and a mucosal absorption enhancer to a subject in need thereof, where the local anesthetic agent and mucosal absorption enhancer are present in an amount sufficient to anesthetize nerve tissue in the trigeminal nerve pathway upon administration. Also provided herein are methods of locally and specifically anesthetizing nerve tissue in the trigeminal nerve pathway and methods of performing a surgical procedure on a subject in need thereof that include intranasally administering a pharmaceutical composition that includes a local anesthetic agent in its free-base form to a subject in need thereof, where the free-base form of the local anesthetic agent is present in an amount sufficient to anesthetize nerve tissue in the trigeminal nerve pathway upon administration. Also provided are methods of locally and specifically anesthetizing nerve tissue in the trigeminal nerve pathway and methods of performing a surgical procedure on a subject in need thereof that include intranasally administering (1) a first pharmaceutical composition including a local anesthetic agent and (2) a second pharmaceutical composition including a mucosal absorption agent, where the local anesthetic agent and the mucosal absorption agent are present in amounts sufficient to anesthetize nerve tissue in the trigeminal nerve pathway upon administration. Also provided are methods of locally and specifically anesthetizing nerve tissue in the trigeminal nerve pathway and methods of performing a surgical procedure on a subject in need thereof that includes intranasally administering (1) a first pharmaceutical composition including a mucosal absorption enhancer and (2) a second pharmaceutical composition including a local anesthetic agent, where the mucosal absorption enhancer and the local anesthetic agent are present in amounts sufficient to anesthetize nerve tissue in the trigeminal nerve pathway upon administration.

The methods and pharmaceutical compositions provided herein allow for a substantial increase (e.g., an increase of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%) in the absorption (e.g., paracellular transport and/or transcellular transport) of local anesthetic agent(s) across the nasal mucosa (e.g., a level of transport sufficient to anesthetize nerve tissue in the trigeminal nerve pathway, e.g., the mandibular nerve) (e.g., as compared to the absorption (e.g., paracellular transport and/or transcellular transport) achieved following intranasal administration of a composition that includes the local anesthetic agent(s) but does not include a mucosal absorption enhancer) and/or provides for a reduction (e.g., a reduction of at least 5%, at least 10%, at least 15%, at least 20%, at least 25%, at least 30%, at least 35%, at least 40%, at least 45%, at least 50%, at least 55%, at least 60%, at least 65%, or at least 70%) in the administered dose (e.g., a safer and lower effective dose) of local anesthetic agent sufficient to anesthetize nerve tissue in the trigeminal nerve pathway (e.g., as compared to intranasal administration of a composition including a local anesthetic agent(s) and not including a mucosal absorption enhancer) and/or allows for a substantial increase (e.g., an increase of one, two, three, four, five, six, seven, eight, nine, or ten) in the number of nerve(s) within the trigeminal nerve pathway that are anesthetized by intranasal delivery (e.g., as compared to intranasal administration of a composition including a local anesthetic agent(s) and not including a mucosal absorption enhancer or administration of a composition including a local anesthetic agent(s) and not including a mucosal absorption enhancer by injection into tissue proximal to the posterior superior alveolar nerve and/or the inferior alveolar nerve). For example, the methods and pharmaceutical compositions provided herein increase the percentage (e.g., an increase of at least 10%, at least 20%, at least 30%, at least 40%, at least 50%, at least 60%, at least 70%, at least 80%, at least 90%, or at least 100%) of the local anesthetic agent (e.g., a protonated form of the local anesthetic agent) that penetrates the nasal mucosa, and that successfully anesthetizes nerve tissue in the trigeminal nerve pathway (e.g., any of the nerve tissues present in the trigeminal nerve pathway described herein) (e.g., as compared to the percentage of the local anesthetic agent that penetrates the nasal mucosa when a composition including the local anesthetic agent(s) and not including a mucosal absorption enhancer is intranasally administered).

As can be appreciated in the art, the various aspects described below can be used in any combination without limitation.

### Subjects

In any of the methods described herein, the subject is in need of oral surgery or a dental procedure. For example, the subject can have an impacted tooth (e.g., a molar), temporomandibular joint (TMJ) problems, cleft palate, cleft lip, an abscess in the oral cavity, or an unequal jaw. Oral surgery can include the extraction of a tooth (e.g., a molar), the positioning and placement of a dental implant into the subject's oral cavity, or the movement or repositioning of all or a part of the upper jaw, lower jaw, or both into a new position that is more balanced and functional. In some examples, the subject is a subject in need of a dental procedure (e.g., the adjustment of braces, the filling of a cavity, a root canal, or the placement of a crown).

In some embodiments of any of the methods described herein, the subject can be in need of ocular or eyelid surgery. A subject can be, e.g., in need of laser eye surgery, cataract surgery, glaucoma surgery (e.g., canaloplasty), refractive surgery (e.g., keratomilleusis, automated lamellar keratoplasty, laser-assisted in situ keratomileusis, photorefractive keratectomy, laser thermal keratoplasty, conductive keratoplasty, limbal relaxing incisions, astigmatic keratotomy, radial keratotomy, mini asymmetric radial keratotomy, hexagonal keratotomy, epikeratophakia, intracorneal rings, implantable contact lenses, presbyopia reversal, anterior ciliary sclerotomy, laser reversal of presbyopia, scleral expansion bands, Karmra inlay, or scleral reinforcement surgery), corneal surgery (e.g., corneal transplant surgery, penetrating keratoplasty, keratoprosthesis, phototherapeutic keratectomy, pterygium excision, corneal tattooing, or Osteo-Odonto-Keratoprosthesis), vitreo-retinal surgery (e.g., vitrectomy, pan retinal photocoagulation, ignipuncture, laser photocoagulation, pneumatic retinoplexy, retinal cryopexy, macular hole repair, partial lamellar sclerouvectomy, partial lamellar sclerocyclochoroidectomy, partial lamellar scherlochoroidectomy, or posterior sclerotomy), eye muscle surgery (e.g., myectomy, myotomy, tenectomy, tenotomy, resection, tucking, or adjustable suture surgery), orbital surgery (e.g., orbital reconstruction), surgery involving the lacrimal apparatus, or eye removal. In some examples, the subject can be in need of eyelid surgery (e.g., blepharoplasty, ptosis repair, ectropion repair, entropion repair, canthectomy, cantholysis, canthopexy, cantoplasty, cantorrhaphy, canthotomy, epicanthoplasty, or tarsorrhaphy). In some examples, a subject can be in need of an eye or eyelid procedure (e.g., ophthalmic examination, removal or a foreign object, and dilation or treatment of blepharitis).

In some examples, the subject can be in need of exterior nose surgery (e.g., rhinoplasty or the removal of a melanoma or nasal cancer), sinus surgery (e.g., removal of tissue or acclusions from a sinus), or a sinus procedure (e.g., the implantation of a drug or medical device in a sinus). In some examples, the subject is about to receive (e.g., within about 1 hour, within about 50 minutes, within about 45 minutes, within about 40 minutes, within about 35 minutes, within about 30 minutes, within about 25 minutes, within about 20 minutes, within about 15 minutes, within about 10 minutes, or within about 5 minutes), e.g., oral surgery, a dental procedure, ocular surgery, eyelid surgery, an ocular procedure, an eyelid procedure, exterior nose surgery, sinus surgery, or a sinus procedure.

In some embodiments, the subject is a female (e.g., a pregnant female). In some embodiments, the subject is a male. For example, a subject in any of the methods described herein can be a child, an adolescent, a teenager, or an adult (a subject that greater than 18 years old, e.g., greater than 20 years old, greater than 25 years old, greater than 30 years old, greater than 35 years old, greater than 40 years old, greater than 45 years old, greater than 50 years old, greater than 55 years old, greater than 60 years old, greater than 65 years old, greater than 70 years old, greater than 75 years old, greater than 80 years old, greater than 90 years old, or greater than 100 years old).

### Trigeminal Nerve Pathway

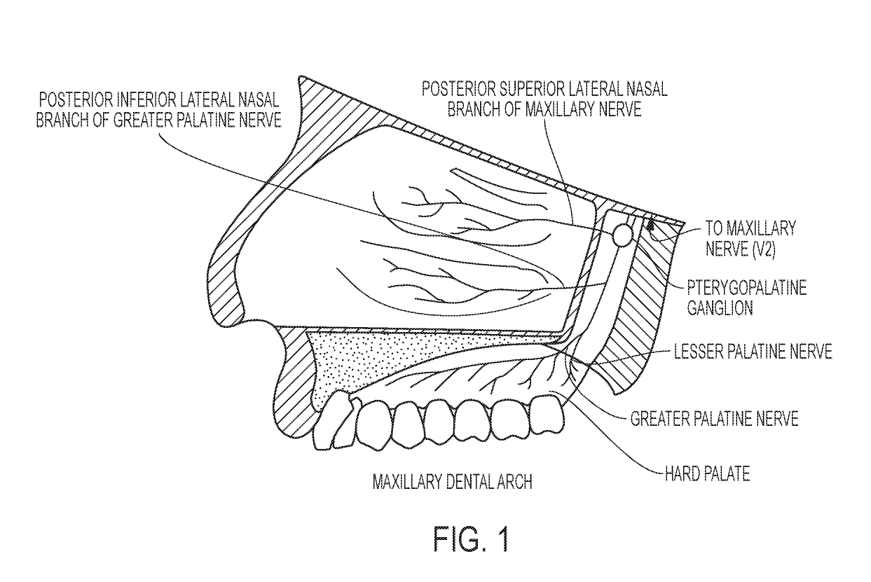

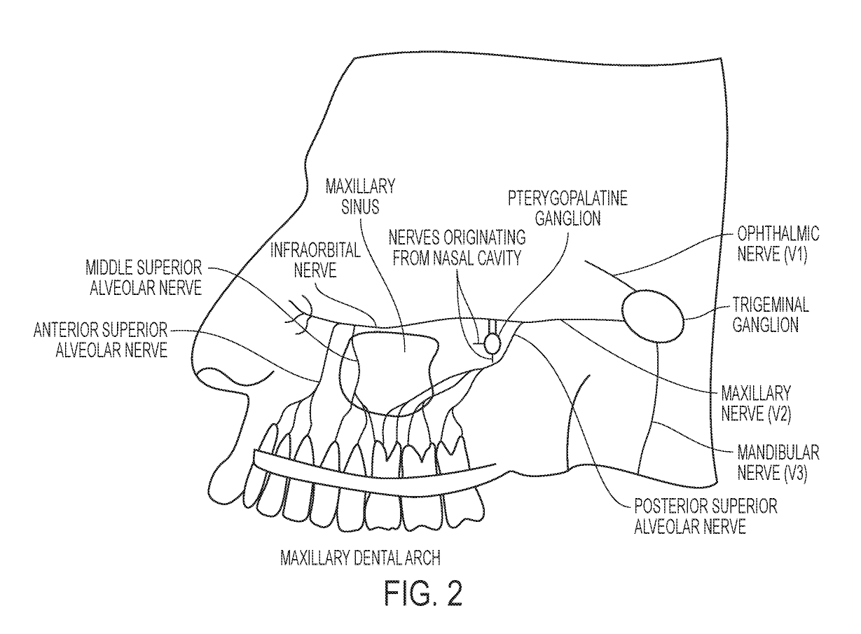

The trigeminal nerve pathway and the nerve tissues included within the trigeminal nerve pathway are well known in the art. For example, a diagram showing non-limiting examples of nerve tissues present in the trigeminal nerve pathway are shown in FIG. 1 and FIG. 2. The trigeminal nerve pathway includes nerve tissue that innervates, e.g., the maxillary dental arch, the mandibular dental arch, the molar teeth and the tissue surrounding the molar teeth, the eyelid, the eye (and eye socket and glandular tissue surrounding the eye), the sinuses, and the nose. The trigeminal nerve pathway includes the trigeminal ganglion which branches into the ophthalmic nerve, the maxillary nerve, and the mandibular nerve. The ophthalmic nerve, the maxillary nerve, and the mandibular nerve further branch into other nerve tissues which go on to innervate one or more of, e.g., the maxillary dental arch, the mandibular dental arch, the molar teeth and the tissue surrounding the molar teeth, the eyelid, the eye (and eye socket and glandular tissue surrounding the eye), the sinuses, and the nose. For example, the mandibular nerve and/or maxillary nerve innervates the molar teeth and tissues surrounding the molar teeth.

In any of the methods described herein, the nerve tissue in the trigeminal nerve pathway that is anesthetized can be one or more of the following nerve tissues (in any combination): trigeminal ganglion, the frontal nerve, the supraorbital nerve, the supratrochlear nerve, the lacrimal nerve, the nasociliary nerve, the infratrochlear nerve, the ciliary nerve, the anterior ethmoidal nerve, the external nasal nerve, the internal nasal nerve, the ophthalmic nerve (V1), the infraorbital nerve, the anterior superior alveolar nerve, the middle superior alveolar nerve, the infraorbital nerve entering the infraorbital canal, the posterior superior alveolar nerve, the ganglionic branches to pterygopalatine ganglion, the pterygopalatine ganglion, the greater and lesser palantine nerves, the lateral nasal branches of the greater palatine nerve, nerve of the pterygoid canal, the nasopalatine nerve, the zygomatic nerve, the zygomaticofacial nerve, the zygomaticotemporal nerve, the lateral nasal branches of the maxillary nerve, the maxillary nerve (V2), the buccal nerve, the auriculotemporal nerve, the lingual neve, the inferior alveolar nerve, the mylohyoid nerve, the mental nerve, the incisive nerve, and the mandibular nerve (V3). For example, any of the methods described herein result in the anesthetization of the mandibular nerve (V3).

### Intranasal Administration

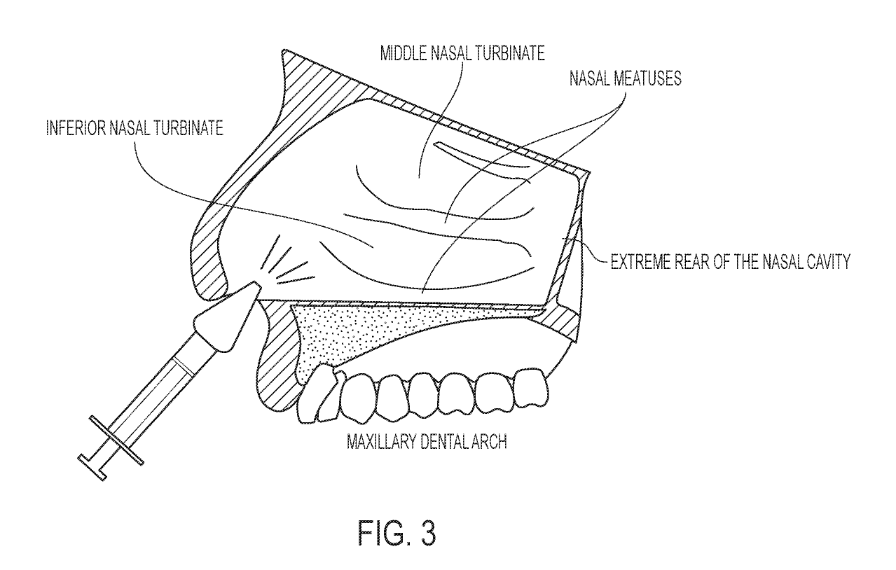

As is known in the art, intranasal administration of a pharmaceutical composition includes placing the pharmaceutical composition on an area of the nasal mucosa. For example, intranasal administration can include the placement (e.g., by spraying an aerosol using an nebulizer, spreading a gel or cream, or providing a liquid solution) of a pharmaceutical composition (e.g., any of the pharmaceutical compositions, any of the first pharmaceutical compositions, and any of the second pharmaceutical compositions described herein) on one of more nasal tissues selected from the group consisting of: the inferior nasal turbinate, the middle nasal turbinate, the nasal meatuses, the extreme rear of the nasal cavity, and the maxillary sinus. For example, in any of the methods described herein, the pharmaceutical composition (e.g., any of the pharmaceutical compositions, any of the first pharmaceutical compositions, and any of the second pharmaceutical compositions described herein) is directly administered onto one or more nasal tissues selected from the group consisting of: the inferior nasal turbinate, the middle nasal turbinate, the nasal meatuses, the extreme rear of the nasal cavity, and the maxillary sinus. FIGS. 3 illustrates non-limiting examples of nasal tissue that any of the pharmaceutical compositions included herein can be placed and/or directly administered onto. The opening to the maxillary sinus is located on the lateral wall of the nasal cavity.

A subject can, e.g., receive one intranasal administration of any of the pharmaceutical compositions, the first pharmaceutical compositions, or the second pharmaceutical compositions described herein. A subject can receive two or more intranasal administrations of any of the pharmaceutical compositions, any of the first pharmaceutical compositions, and/or any of the second pharmaceutical compositions described herein. For example, when a subject receives two or more doses, the doses can be intranasally administered, e.g., at a time interval of between about 1 minute to about 10 minutes (e.g., about 1 minute to about 8 minutes, about 1 minute to about 6 minutes, at about 1 minute to about 4 minutes, or at about 1 minute to about 2 minutes). In some embodiments, a subject can be intranasally administered at least one dose of any of the pharmaceutical compositions, any of the first pharmaceutical compositions, and/or any of the second pharmaceutical compositions described herein, prior to the start of a surgery or a procedure (e.g., any of the exemplary types of surgery or any of the exemplary types of procedures described herein) and is intranasally administered one or more additional doses of any of the pharmaceutical compositions, any of the first pharmaceutical compositions, and/or any of the second pharmaceutical compositions described herein, during the middle of the procedure. The one or more doses of the pharmaceutical composition, the first pharmaceutical composition, and/or the second pharmaceutical composition can be identical or different (e.g., in the total amount of local anesthetic agent delivered or different pharmaceutical compositions). The one or more doses of the pharmaceutical composition, the first pharmaceutical composition, or the second pharmaceutical composition can be administered as different formulations (e.g., a first dose of a pharmaceutical composition, a first pharmaceutical composition, or a second pharmaceutical composition, can be intranasally administered as a gel and one or more additional doses of a pharmaceutical composition, a first pharmaceutical composition or a second pharmaceutical composition, can be intranasally administered as a nasal spray).

In any of the methods described herein, the pharmaceutical composition, the first pharmaceutical composition, and/or the second pharmaceutical composition (e.g., any of the pharmaceutical compositions, the first pharmaceutical compositions, and the second pharmaceutical compositions described herein) is administered in a volume of between about 10 μL and about 400 μL (e.g., between about 50 μL and about 350 μL, between about 50 μL and about 300 μL, between about 50 μL and about 250 μL, or between about 50 μL and about 200 μL) of solution. In some embodiments of any of the methods described herein, the pharmaceutical composition, the first pharmaceutical composition, and/or the second pharmaceutical composition, is intranasally administered in a spray. For example, the pharmaceutical composition, the first pharmaceutical composition, or the second pharmaceutical composition, can be delivered via an intranasal spray (e.g., the administration of at least one, at least two, at least three, at least four, at least five, two, three, four, or five sprays into each nostril (if administered to both nostrils) or a single nostril (if administered to only one nostril)).

An appropriate dose of any of the pharmaceutical compositions, the first pharmaceutical compositions, and the second pharmaceutical compositions described herein, can be determined by a physician or dental professional (e.g., by assessing different factors including the subject's weight, age, height, sex, and general state of health).

### Pharmaceutical Compositions

### Compositions Comprising a Local Anesthetic Agent and a Mucosal Absorption Enhancer

Provided herein are pharmaceutical compositions that include a local anesthetic agent and a mucosal absorption enhancer, where the composition is formulated for intranasal administration (e.g., as a gel, a solution, a spray, a paste, an emulsion, a powder, a colloid, or a nanoparticle) and the local anesthetic agent and the mucosal absorption enhancer are present in an amount sufficient to anesthetize nerve tissue in the trigeminal nerve pathway in a subject (e.g., a human) upon nasal administration. Also provided are first pharmaceutical compositions that include a local anesthetic agent and second pharmaceutical compositions that include a mucosal absorption enhancer. Also provided are first pharmaceutical compositions that include a mucosal absorption enhancer and second pharmaceutical compositions that include a local anesthetic agent.

Non-limiting examples of local anesthetic agents that can be included in any of the pharmaceutical compositions (or any of the first and/or second pharmaceutical compositions) provided herein are described below. Non-limiting examples of the concentration of local anesthetic agent(s) that can be included in any of the pharmaceutical compositions (or the first and/or second pharmaceutical compositions) provided herein are also described below. Non-limiting examples of mucosal absorption enhancers are described below. Non-limiting examples of the concentration of mucosal absorption enhancer(s) that can be included in any of the pharmaceutical compositions (or the first and second pharmaceutical compositions) provided herein are also described below.

Any of the first and/or second pharmaceutical compositions that include a local anesthetic agent can further include one or more (e.g., two, three, four, or five) of the following: a mucosal absorption enhancer (e.g., any of the exemplary mucosal absorption enhancers described herein or known in the art), a vasoconstrictor (e.g., any of the exemplary vasoconstrictors described herein or known in the art), a preservative (e.g., any of the exemplary preservatives described herein or known in the art), a viscosity enhancing agent (e.g., any of the exemplary viscosity enhancing agents described herein or known in the art), and an antihistamine (e.g., any of the exemplary antihistamines described herein or known in the art). Any of the first and/or second pharmaceutical compositions that include a mucosal absorption enhancer can further include one or more (e.g., two, three, four, or five) of the following: a local anesthetic agent (e.g., any of the local anesthetic agents described herein or known in the art), a vasoconstrictor (e.g., any of the exemplary vasoconstrictors described herein or known in the art), a preservative (e.g., any of the exemplary preservatives described herein or known in the art), a viscosity enhancing agent (e.g., any of the exemplary viscosity enhancing agents described herein or known in the art), and an antihistamine (e.g., any of the exemplary antihistamines described herein or known in the art).

Any of the pharmaceutical compositions, first pharmaceutical compositions, and/or second pharmaceutical compositions described herein can further include a vasoconstrictor. Non-limiting examples of vasoconstrictor(s) that can be included in any of the pharmaceutical compositions, first pharmaceutical compositions, and/or second pharmaceutical compositions provided herein are also described below. Non-limiting examples of the concentration of vasoconstrictor(s) that can be included in any of the pharmaceutical compositions, first pharmaceutical compositions, and/or second pharmaceutical compositions provided herein are also described below.

Some examples of any of the pharmaceutical compositions, first pharmaceutical compositions, and/or second pharmaceutical compositions described herein can further include a preservative and/or a viscosity enhancing agent. Non-limiting examples of preservative(s) that can be included in any of the pharmaceutical compositions, first pharmaceutical compositions, and/or second pharmaceutical compositions provided herein are also described below. Non-limiting examples of the concentrations of preservative(s) that can be included in any of the pharmaceutical compositions, first pharmaceutical compositions, and/or second pharmaceutical compositions provided herein are also described below. Non-limiting examples of viscosity enhancing agent(s) that can be included in any of the pharmaceutical compositions, first pharmaceutical compositions, and/or second pharmaceutical compositions provided herein are described below. Non-limiting examples of the concentrations of viscosity enhancing agent(s) that can be included in any of the pharmaceutical compositions, first pharmaceutical compositions, and/or second pharmaceutical compositions provided herein are also described below.

Some examples of the pharmaceutical compositions, first pharmaceutical compositions, and/or second pharmaceutical compositions provided herein further include a pharmaceutically acceptable carrier(s) and/or an antihistamine. Non-limiting examples of such pharmaceutically acceptable carrier(s) that can be included in any of the pharmaceutical compositions, first pharmaceutical compositions, and/or second pharmaceutical compositions provided herein are described below. Non-limiting examples of antihistamine(s) that can be included in any of the pharmaceutical compositions, first pharmaceutical compositions, and/or second pharmaceutical compositions provided here are described below. Non-limiting examples of concentrations of antihistamine(s) that can be included in any of the pharmaceutical compositions, first pharmaceutical compositions, and/or second pharmaceutical compositions provided herein are also described below.

In some examples of any of the pharmaceutical compositions, first pharmaceutical compositions, and/or second pharmaceutical compositions provided herein, the composition includes tetracaine as the local anesthetic agent and optionally, chitosan (e.g., chitosan hydrochloride or chitosan glutamate) or sodium caprate as the mucosal absorption enhancer.

The pH of the pharmaceutical compositions, first pharmaceutical compositions, and/or second pharmaceutical compositions can be adjusted to that corresponding to the pH of the nasal mucosa in a subject (e.g., a human). For example, any of the pharmaceutical compositions, first pharmaceutical compositions, and/or second pharmaceutical compositions provided herein have a pH of between about 4.0 and about 9.0 (e.g., a pH of between about 4.0 and about 8.5, a pH of between about 4.0 and about 8.0, a pH of between about 4.0 and about 7.5, a pH of between about 4.0 and about 7.0, a pH of between about 4.0 and about 6.5, a pH of between about 4.5 and about 6.5, a pH of between about 4.5 and about 6.0, a pH of between about 5.0 and about 6.5, or a pH of between about 5.5 and about 6.5).

Some examples of any of the pharmaceutical compositions, first pharmaceutical compositions, and/or second pharmaceutical compositions provided herein have a certain particle size (e.g., particles having an average diameter of between about 8 μm and about 50 μm) (e.g., between about 10 μm and about 45 μm, between about 10 μm and about 40 μm, between about 10 μm and about 35 μm, between about 10 μm and about 30 μm, between about 10 μm and about 25 μm, between about 10 μm and about 20 μm, between about 10 μm and about 15 μm, or between about 10 μm and about 50 μm) (e.g., when delivered to the target tissue, e.g., nasal mucosa). In some embodiments, at least 10% (e.g., at least 20%, at least 25%, at least 30%, at least 35%, at least 40%, at least 45% at least 50%, at least 55%, at least 60%, at least 65%, at least 70%, at least 75% at least 80%, at least 85%, or at least 90%) of the microparticles present in the pharmaceutical composition, the first pharmaceutical composition, and/or second pharmaceutical composition (e.g., any of the pharmaceutical compositions, first pharmaceutical compositions, and second pharmaceutical compositions provided herein) have an average diameter greater than or equal to 10 μm (e.g., greater than or equal to 15 μm, or greater than or equal to 20 μm).

A non-limiting example of a pharmaceutical composition provided herein is shown in Table 7. Additional examples of a pharmaceutical composition provided herein include: between about 1% to about 10% (e.g., between about 1% and about 8%, between about 1% and about 6%, between about 1% and about 5%, between about 1% and about 4%, or between about 2% and about 4%) % weight/volume tetracaine (e.g., tetracaine hydrochloride), and between about 0.1% and about 5% (e.g., between about 0.1% and about 4.5%, between about 0.1% and about 4.0%, between about 0.1% and about 3.5%, between about 0.1% and about 3.0%, between about 0.1% and about 2.5%, between about 0.1% and about 2.0%, between about 0.1% and about 1.5%, between about 0.1% and about 1.0%, or between about 0.3% and about 0.7%, or between about 0.1% and about 0.5%) % weight/volume sodium caprate, and optionally one or more of: between about 0.01% to about 2.0% (e.g., between about 0.01% to about 1.5%, between about 0.01% to about 1.0%, between about 0.01% to about 0.5%, between about 0.01% to about 0.1%, or between about 0.03% to about 0.07%) % weight/volume oxymetazoline (e.g., oxymetazoline hydrochloride); between about 0.1% to about 10% (e.g., between about 0.1% to about 8%, between about 0.1% and about 6%, between about 0.1% and about 4%, between about 0.1% and about 3%, between about 0.1% and about 2%, between about 0.1% and about 1.0%, or between about 0.5% and about 1.5%) % weight/volume of citric acid (e.g., citric acid anhydrous); between about 0.5% to about 5% (e.g., between about 0.5% to about 4.5%, between about 0.5% to about 4.0%, between about 0.5% to about 3.5%, between about 0.5% to about 3.0%, between about 0.5% to about 2.5%, between about 0.5% to about 2.0%, between about 0.5% to about 1.5%, or between about 0.7% to about 1.2%) % weight/volume of benzyl alcohol, and a balance of purified water.

A non-limiting example of a pharmaceutical composition provided herein is shown in Table 3. Additional examples of a pharmaceutical composition provided herein include: between about 1% to about 10% (e.g., between about 1% and about 8%, between about 1% and about 6%, between about 1% and about 5%, between about 1% and about 4%, or between about 2% and about 4%) % weight/volume tetracaine (e.g., tetracaine hydrochloride) and between about 0.1% to about 5% (e.g., between about 0.1% and about 4.5%, between about 0.1% and about 4.0%, between about 0.1% and about 3.5%, between about 0.1% and about 3.0%, between about 0.1% and about 2.5%, between about 0.1% and about 2.0%, between about 0.1% and about 1.5%, between about 0.1% and about 1.0%, between about 0.3% and about 0.7%, or between about 0.1% and about 0.5%) % weight/volume chitosan (e.g., chitosan hydrochloride), and optionally one or more of: between about 0.01% to about 2.0% (e.g., between about 0.01% to about 1.5%, between about 0.01% to about 1.0%, between about 0.01% to about 0.5%, between about 0.01% to about 0.1%, or between about 0.03% to about 0.07%) % weight/volume oxymetazoline (e.g., oxymetazoline hydrochloride); between about 0.1% to about 10% (e.g., between about 0.1% to about 8%, between about 0.1% and about 6%, between about 0.1% and about 4%, between about 0.1% and about 3%, between about 0.1% and about 2%, between about 0.1% and about 1.0%, or between about 0.5% and about 1.5%) % weight/volume of citric acid (e.g., citric acid anhydrous); between about 0.5% to about 5% (e.g., between about 0.5% to about 4.5%, between about 0.5% to about 4.0%, between about 0.5% to about 3.5%, between about 0.5% to about 3.0%, between about 0.5% to about 2.5%, between about 0.5% to about 2.0%, between about 0.5% to about 1.5%, or between about 0.7% to about 1.2%) % weight/volume of benzyl alcohol, and a balance of purified water.

A non-limiting example of a pharmaceutical composition provided herein is shown in Table 9. Additional examples of a pharmaceutical composition provided herein include: between about 1% to about 10% (e.g., between about 1% and about 8%, between about 1% and about 6%, between about 1% and about 5%, between about 1% and about 4%, or between about 2% and about 4%) % weight/volume tetracaine in its free-base form and between about 0.1% to about 50% (e.g., between about 0.1% and about 40%, between about 0.1% and about 30.0%, between about 1.0% and about 20%, between about 3.0% and about 18.0%, between about 4.0% and about 16.0%, between about 5.0% and about 15.0%, between about 5.0% and about 14.0%, between about 6.0% and about 10.0%, or between about 6.0% and about 8.0%) % weight/volume DOPC, and between about 0.1% to about 12.5% (e.g., between about 0.1% and about 10.0%, between about 1.0% and about 8.0%, between about 1.0% and about 6.0%, between about 1.0% and about 5.0%, between about 1.0% and about 4.0%, between about 1.0% and about 3.0%, between about 1.0% and about 2.0%) % weight/volume cholesterol, and optionally one or more of: between about 0.1% to about 10% (e.g., between about 0.1% to about 8%, between about 0.1% and about 6%, between about 0.1% and about 4%, between about 0.1% and about 3%, between about 0.1% and about 2%, between about 0.1% and about 1.0%, or between about 0.5% and about 1.5%) % weight/volume of citric acid (e.g., citric acid anhydrous); between about 0.5% to about 5% (e.g., between about 0.5% to about 4.5%, between about 0.5% to about 4.0%, between about 0.5% to about 3.5%, between about 0.5% to about 3.0%, between about 0.5% to about 2.5%, between about 0.5% to about 2.0%, between about 0.5% to about 1.5%, or between about 0.7% to about 1.2%) % weight/volume of benzyl alcohol, and a balance of purified water.

Non-limiting examples of pharmaceutical compositions provided herein are shown in Table 4, Table 5, Table 6, Table 8, Table 10, and Table 11.

### Compositions Comprising a Local Anesthetic Agent in its Free-Base Form

Also provided herein are pharmaceutical compositions that include a local anesthetic agent in its free-base form, where the composition is formulated for intranasal administration (e.g., as a gel, a solution, a spray, a paste, or a powder) and the free-base form of the local anesthetic agent is present in an amount sufficient to anesthetize nerve tissue in the trigeminal nerve pathway in a subject (e.g., a human) upon nasal administration. Non-limiting examples of local anesthetic agents that can be included in any of the pharmaceutical compositions provided herein are described below. Non-limiting examples of the concentration of local anesthetic agent(s) that can be included in any of the pharmaceutical compositions provided herein are also described below.

Any of the pharmaceutical compositions can further include one or more of a drug delivery vehicle (e.g., any of the exemplary drug delivery vehicles described herein or known in the art), a preservative (e.g., any of the exemplary preservatives described herein or known in the art), a viscosity enhancing agent (e.g., any of the exemplary viscosity enhancing agents described herein or known in the art), an antihistamine (e.g., any of the exemplary antihistamines described herein or known in the art), and a mucosal absorption enhancer (e.g., any of the exemplary mucosal absorption enhancers described herein or known in the art). For example, in any of the pharmaceutical compositions that further include a drug delivery vehicle, the drug delivery vehicle can encapsulate or entrap the local anesthetic agent in its free-base form.

In some examples, the drug delivery vehicle is a microemulsion (e.g., an oil-in-water microemulsion including an aqueous phase, a lipid phase, and a surfactant). In some examples, a microemulsion can further include a co-surfactant. In some embodiments, the aqueous phase in a microemulsion can include at least about 10% (e.g., at least 12%, at least 15%, at least 20%, or at least 25%) by weight of the pharmaceutical composition. In some examples, the lipid phase comprises about 0.1% to about 50% (e.g., about 0.1% to about 40%, about 0.1% to about 30%, about 0.1% to about 30%, between about 0.1% to about 25%, between about 0.1% to about 20%, between about 1% to about 50%, between about 1% to about 40%, between about 1% to about 30%, between about 1% to about 25%, between about 1% to about 20%, between about 5% to about 50%, between about 5% to about 40%, between about 5% to about 30%, between about 5% to about 25%, or between about 5% to about 20%) by weight of the pharmaceutical composition. In some examples, one or both of the surfactant and co-surfactant each makes-up about 0.1% to about 80% by weight of the pharmaceutical composition (e.g., between about 0.1% to about 70%, between about 0.1% to about 65%, between about 0.1% to about 60%, between about 0.1% to about 50%, between about 0.1% to about 45%, between about 0.1% to about 40%, between about 0.1% to about 35%, between about 0.1% to about 30%, or between about 0.1% to about 20% by weight of the pharmaceutical composition). In some examples, the lipid to total surfactant (the surfactant alone or the surfactant plus the co-surfactant (if present in the pharmaceutical composition) ratio in the pharmaceutical composition is between about 1:1 to about 1:10 (e.g., between about 1:1 to about 1:9, between about 1:1 to about 1:8, between about 1:1 to about 1:7, between about 1:1 to about 1:6, between about 1:1 to about 1:5, between about 1:1 to about 1:4, or between about 1:1 to about 1:3). In some examples, the surfactant to co-surfactant (if present in the pharmaceutical composition) ratio in the pharmaceutical composition is between about 6:1 to about 1:6 (e.g., between about 5:1 to about 1:6, between about 4:1 to about 1:6, between about 3:1 to about 1:6, between about 1:1 to about 1:6, between about 1:1 to about 1:4, between about 1:1 to about 1:3, or between about 1:1 to about 1:2). In some examples, the lipid is selected from the group of castor oil, corn oil, cottonseed oil, olive oil, peanut oil, soybean oil, vegetable oil, coconut oil, isopropyl palmitate, light mineral oil, oleic acid, medium chain mono/diglycerides, and propylene glycol dicaprylocaprate. In some examples, the lipid is a local anesthetic agent in its oil form. In some examples, the surfactant and/or co-surfactant is selected from the group of: the surfactant and/or the co-surfactant is selected from the group consisting of: polysorbate 20, polysorbate 40, polysorbate 60, polysorbate 65, polysorbate 80, polyoxyl 8 stearate, polypropylene glycol, propylene glycol, carbomer 1342, carbomer 934, carbomer 934P, carbomer 940, carbomer 941, carbomer 974, carbomer 980, carbomer 981, oleyl polyethylene glycol glyceride, PEG-300, PEG-400, PEG-3500, poloxamer 124, poloxamer 188, poloxamer 407, polyoxyl 35 castor oil, polyoxyl 40 hydrogenated castor oil, polyoxyl 40 stearate, polyoxyl 6 and polyoxyl 32 palmitostearate, polyoxyl 8 stearate, polyoxyl glyceryl stearate, polyoxyl lanolin, polyoxyl palmitate, polyoxyl stearate, glyceryl oleate, propylene carbonate, sorbitan monooleate, glycerin, PEG-5 oleate, sodium N-methyl N-cocoyl taurate, PEG-8 caprylic/capric gylcerides, and diethylene glycol monoethyl ether.

Some embodiments of any of the pharmaceutical compositions described herein can optionally further include a vasoconstrictor (e.g., oxymetazoline, phenylephrine, naphazoline, propylhexadrine, levodesoxyephedrine, epinephrine, norepinephrine, and a salt thereof, or any other vasoconstrictor known in the art) (e.g., the vasoconstrictor comprising about 0.001% to about 10% (e.g., between about 0.001% to about 5%, between about 0.001% to about 1%, between about 0.001% to about 0.5%, between about 0.001% to about 0.25%, or between about 0.001% to about 0.1% by weight of the composition. Some embodiments of any of the pharmaceutical compositions described herein do not include a vasoconstrictor.

Some examples of any of the pharmaceutical compositions described herein can further include a preservative and/or a viscosity enhancing agent. Non-limiting examples of preservative(s) that can be included in any of the pharmaceutical compositions provided herein are also described below. Non-limiting examples of the concentrations of preservative(s) that can be included in any of the pharmaceutical compositions provided herein are also described below. Non-limiting examples of viscosity enhancing agent(s) that can be included in any of the pharmaceutical compositions provided herein are described below. Non-limiting examples of the concentrations of viscosity enhancing agent(s) that can be included in any of the pharmaceutical compositions provided herein are also described below.

Some examples of the compositions provided herein further include a pharmaceutically acceptable excipient(s) and/or an antihistamine. Non-limiting examples of such pharmaceutically acceptable excipient(s) that can be included in any of the pharmaceutical compositions provided herein are described below. Non-limiting examples of antihistamine(s) that can be included in any of the pharmaceutical compositions provided here are described below. Non-limiting examples of concentrations of antihistamine(s) that can be included in any of the pharmaceutical compositions provided herein are also described below.

Some examples of the compositions provided herein further include a mucosal absorption enhancer. Non-limiting examples of mucosal absorption enhancer(s) that can be included in any of the pharmaceutical compositions provided here are described below. Non-limiting examples of concentrations of mucosal absorption enhancer(s) that can be included in any of the pharmaceutical compositions provided herein are also described below.

Some examples of any of the pharmaceutical compositions provided herein include tetracaine in its free-base form as the local anesthetic agent in its free-base form and multilamellar liposome vesicles as the drug delivery vehicle, where the multilamellar liposome vesicles encapsulate and/or entrap the tetracaine in its free-base form.

The pH of the pharmaceutical composition can be adjusted to that corresponding to the pH of the nasal mucosa in a subject (e.g., a human). For example, any of the pharmaceutical composition provided herein have a pH of between about 4.0 and about 9.0 (e.g., a pH of between about 4.9 and about 8.5, a pH of between about 4.0 and about 8.0, a pH of between about 4.0 and about 7.5, a pH of between about 4.0 and about 7.0, a pH of between about 4.0 and about 6.5, a pH of between about 4.5 and about 6.5, a pH of between about 4.5 and about 6.0, a pH of between about 5.0 and about 6.5, or a pH of between about 5.5 and about 6.5).

Some examples of any of the pharmaceutical compositions provided herein have a certain particle size (e.g., particles having an average diameter of between about 8 μm and about 50 μm) (e.g., between about 10 μm and about 45 μm, between about 10 μm and about 40 μm, between about 10 μm and about 35 μm, between about 10 μm and about 30 μm, between about 10 μm and about 25 μm, between about 10 μm and about 20 μm, between about 10 μm and about 15 μm, or between about 10 μm and about 50 μm) (e.g., when delivered to the target tissue, e.g., nasal mucosa). In some embodiments, at least 10% (e.g., at least 20%, at least 25%, at least 30%, at least 35%, at least 40%, at least 45% at least 50%, at least 55%, at least 60%, at least 65%, at least 70%, at least 75% at least 80%, at least 85%, or at least 90%) of the microparticles present in the pharmaceutical composition (e.g., any of the pharmaceutical compositions provided herein) have an average diameter greater than or equal to 10 μm (e.g., greater than or equal to 15 μm, or greater than or equal to 20 μm). Some examples of the pharmaceuticals compositions that include a drug delivery vehicle can include liposomes and/or micelles having any of the exemplary size ranges described herein. Some examples of the pharmaceuticals compositions that include a drug delivery vehicle can include lipid nanoparticles having any of the exemplary size ranges described herein. Some of the pharmaceutical compositions that include a drug delivery vehicle can include emulsions and/or microemulsions that have droplets having any of the exemplary size ranges described herein.

A non-limiting example of a pharmaceutical composition provided herein is shown in Table 5. Additional examples of a pharmaceutical composition provided herein include: between about 1% to about 10% (e.g., between about 1% and about 8%, between about 1% and about 6%, between about 1% and about 5%, between about 1% and about 4%, or between about 2% and about 4%) % weight/volume tetracaine in its free-base form and between about 0.1% to about 50% (e.g., between about 0.1% and about 40%, between about 0.1% and about 30.0%, between about 1.0% and about 20%, between about 3.0% and about 18.0%, between about 4.0% and about 16.0%, between about 5.0% and about 15.0%, between about 5.0% and about 14.0%, between about 6.0% and about 10.0%, or between about 6.0% and about 8.0%) % weight/volume DOPC and between about 0.1% to about 12.5% (e.g., between about 0.1% and about 10.0%, between about 1.0% and about 8.0%, between about 1.0% and about 6.0%, between about 1.0% and about 5.0%, between about 1.0% and about 4.0%, between about 1.0% and about 3.0%, between about 1.0% and about 2.0%) % weight/volume cholesterol and optionally one or more of: between about 0.1% to about 10% (e.g., between about 0.1% to about 8%, between about 0.1% and about 6%, between about 0.1% and about 4%, between about 0.1% and about 3%, between about 0.1% and about 2%, between about 0.1% and about 1.0%, or between about 0.5% and about 1.5%) % weight/volume of citric acid (e.g., citric acid anhydrous); between about 0.5% to about 5% (e.g., between about 0.5% to about 4.5%, between about 0.5% to about 4.0%, between about 0.5% to about 3.5%, between about 0.5% to about 3.0%, between about 0.5% to about 2.5%, between about 0.5% to about 2.0%, between about 0.5% to about 1.5%, or between about 0.7% to about 1.2%) % weight/volume of benzyl alcohol, and a balance of purified water.

### Local Anesthetic Agents

Non-limiting examples of local anesthetic agent(s) that can be included in any of the pharmaceutical compositions provided herein, or any of the first pharmaceutical compositions and/or second pharmaceutical compositions provided herein include: benzocaine, butacaine, tetracaine, lidocaine, dyclonine, pramoxine, dibucaine, cocaine, etidocaine, bupivacaine, levobupivacaine, ropivacaine, procaine, chloroprocaine, mepivacaine, prilocaine, articaine, hexylcaine, oxetacaine, and a salt or mineral acid thereof (e.g., as a hydrochloric acid or a sulfuric acid). Additional examples of local anesthetic agents that can be included in any of the pharmaceutical compositions, first pharmaceutical compositions, and /or second pharmaceutical compositions described herein include tetracaine hydrochloride, lidocaine hydrochloride, dyclonine hydrochloride, pramoxine hydrochloride, dibucaine hydrochloride, butacaine sulfate, and cocaine hydrochloride. Additional local anesthetic agents are known in the art.

Local anesthetic agent(s) that can be included in any of the pharmaceutical compositions, first pharmaceutical compositions, and/or second pharmaceutical compositions described herein, are, e.g., at least 10% (e.g., at least 20%, at least 30%, at least 40%, at least 50%, at least 60%, at least 70%, at least 80%, at least 90%, or at least 95%) protonated at a pH of between about 4.0 and about 9.0 (e.g., a pH of between about 4.0 and about 8.5, a pH of between about 4.0 and about 8.0, a pH of between about 4.0 and about 7.5, a pH of between about 4.0 and about 7.0, a pH of between about 4.0 and about 6.5, a pH of between about 4.5 and about 6.5, a pH of between about 4.5 and about 6.0, a pH of between about 5.0 and about 6.5, or a pH of between about 5.5 and about 6.5) (e.g., in a buffered solution (e.g., phosphate buffered solution) or in any of the pharmaceutical compositions, first pharmaceutical compositions, and/or second pharmaceutical compositions described herein).

The concentration of local anesthetic agent(s) in any of the pharmaceutical compositions, first pharmaceutical compositions, and/or second pharmaceutical compositions described herein can be determined by taking into account the specific local anesthetic agent(s) that are included in the composition, as well as the desired strength and activity of the pharmaceutical composition. Any of the local anesthetic agent(s) described herein can be present in the pharmaceutical composition (e.g., a liquid, gel, or paste), first pharmaceutical composition (e.g., a liquid, gel, or paste), and/or second pharmaceutical composition (e.g., a liquid, gel, or paste) at a concentration of between about 0.1% to about 65% (e.g., between about 0.1% to about 60% between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 8%, between about 0.1% and about 6%, between about 0.1% and about 5%, between about 0.1% and about 4%, between about 0.1% and about 3%, between about 0.1% and about 2.0%, between about 0.1% and about 1.0%, between about 0.1% and about 0.5%, between about 0.5% and about 65%, between about 0.5% and about 60%, between about 0.5% and about 55%, between about 0.5% and about 50%, between about 0.5% and about 45%, between about 0.5% and about 40%, between about 0.5% and about 35%, between about 0.5% and about 30%, between about 0.5% and about 25%, between about 0.5% and about 20%, between about 0.5% and about 15%, between about 0.5% and about 10%, between about 0.5% and about 8%, between about 0.5% and about 6.0%, between about 0.5% and about 5.0%, between about 0.5% and about 4.0%, between about 0.5% and about 3.0%, between about 0.5% and about 2.0%, between about 0.5% and about 1.0%, between about 1% and about 60%, between about 1% and about 55%, between about 1% and about 50%, between about 1% and about 45%, between about 1% and about 40%, between about 1% and about 35%, between about 1% and about 30%, between about 1% and about 25%, between about 1% and about 20%, between about 1% and about 15%, between about 1% and about 10%, between about 1% and about 8%, between about 1% and about 6%, between about 1% and about 5%, between about 1% and about 4%, between about 1% and about 3%, between about 1% and about 2.5%, between about 1% and about 2%, or between about 1% and about 1.5%) by weight of the composition (e.g., w/w or w/v).

In some embodiments of any of the pharmaceutical compositions, first pharmaceutical compositions, and/or second pharmaceutical compositions provided herein, the local anesthetic agent is tetracaine. In some examples, the local anesthetic agent included in any of the pharmaceutical compositions, first pharmaceutical compositions, and/or second pharmaceutical compositions provided herein is lipophilic (e.g., tetracaine). In some examples, the local anesthetic agent included in any of the pharmaceutical compositions, first pharmaceutical compositions, and/or second pharmaceutical compositions provided herein has lipophilic properties in its base form and hydrophilic properties in its cationic form. In some examples, the local anesthetic agent included in any of the compositions provided herein is present in its base form (e.g., when packaged in a mucosal absorption enhancer that is a liposome, a microemulsion, a solid lipid nanoparticle, a micelle, or a transferosome).

Pharmaceutical compositions provided herein can be prepared using methods that enrich the presence the local anesthetic agent is in its free-base form in the pharmaceutical composition. For example, the local anesthetic agent (e.g., tetracaine base) can first be dissolved in an adequately lipophilic solvent to ensure that it is encapsulated and/or entrapped in its free-base form by a drug delivery vehicle. For example, when preparing a local anesthetic agent that is entrapped in a liposome in its free-base form, the local anesthetic agent (e.g., tetracaine base) is dissolved in a nonpolar solvent (e.g., chloroform) along with a desired phospholipid (e.g., a phosphatidylcholine), and the nonpolar solvent (e.g. chloroform) is subsequently evaporated (e.g., using rotary evaporation) to produce a layer of dry lipid and local anesthetic agent in its free-base form. Liposomes (e.g., multilamellar vesicles) with a local anesthetic agent in its free-base form (e.g., tetracaine base) entrapped within its lipid bilayer form upon the addition of a pharmaceutically acceptable carrier (e.g., water). Additional methods of liposomal preparation are well known in the art. For example, when formulating a local anesthetic agent in its free-base form entrapped within the oil phase of a microemulsion, the local anesthetic in its free-base form (e.g., tetracaine base) is pre-dissolved in the desired oil (e.g., isopropyl myristate). Additional methods of ensuring the local anesthetic agent is entrapped in its free-base form during production of microemulsions are known in the art. Additional methods of enriching for the presence of a local anesthetic agent in its free-base form when entrapped and/or encapsulated by a drug delivery vehicle (e.g., any of the exemplary drug delivery vehicles described herein or known in the art) are known in the art.

The amount of the free-base form of the local anesthetic agent present in any of the pharmaceutical compositions described herein can be at least 1% (e.g., at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%) of the total amount of the local anesthetic agent present in the pharmaceutical composition. For example, the amount of the free-base form of the local anesthetic agent present in any of the pharmaceutical composition described herein can be between 5% and about 99% (e.g., between about 1% and about 95%, between about 5% and about 90%, between about 5% and about 85%, between about 5% and about 80%, between about 5% and about 80%, between about 5% and about 75%, between about 5% and about 70%, between about 5% and about 65%, between about 5% and about 60%, between about 5% and about 55%, between about 5% and about 50%, between about 5% and about 45%, between about 5% and about 40%, between about 5% and about 35%, between about 5% and about 30%, between about 5% and about 25%, between about 5% and about 20%, between about 5% and about 15%, between about 10% and about 95%, between about 10% and about 90%, between about 10% and about 85%, between about 10% and about 80%, between about 10% and about 75%, between about 10% and about 70%, between about 10% and about 65%, between about 10% and about 60%, between about 10% and about 55%, between about 10% and about 50%, between about 10% and about 45%, between about 10% and about 40%, between about 10% and about 35%, between about 10% and about 30%, between about 10% and about 25%, between about 10% and about 20%, between about 15% and about 95%, between about 15% and about 90%, between about 15% and about 85%, between about 15% and about 80%, between about 15% and about 75%, between about 15% and about 70%, between about 15% and about 65%, between about 15% and about 60%, between about 15% and about 55%, between about 15% and about 50%, between about 15% and about 45%, between about 15% and about 40%, between about 15% and about 35%, between about 15% and about 30%, between about 15% and about 25%, between about 20% and about 95%, between about 20% and about 90%, between about 20% and about 85%, between about 20% and about 80%, between about 20% and about 75%, between about 20% and about 70%, between about 20% and about 65%, between about 20% and about 60%, between about 20% and about 55%, between about 20% and about 50%, between about 20% and about 45%, between about 20% and about 40%, between about 20% and about 35%, between about 20% and about 30%, between about 25% and about 95%, between about 25% and about 90%, between about 25% and about 85%, between about 25% and about 80%, between about 25% and about 75%, between about 25% and about 70%, between about 25% and about 65%, between about 25% and about 60%, between about 25% and about 55%, between about 25% and about 50%, between about 25% and about 45%, between about 25% and about 40%, between about 25% and about 35%, between about 30% and about 95%, between about 30% and about 90%, between about 30% and about 85%, between about 30% and about 80%, between about 30% and about 75%, between about 30% and about 70%, between about 30% and about 65%, between about 30% and about 60%, between about 30% and about 55%, between about 30% and about 50%, between about 30% and about 45%, between about 30% and about 40%, between about 35% and about 95%, between about 35% and about 90%, between about 35% and about 85%, between about 35% and about 80%, between about 35% and about 75%, between about 35% and about 70%, between about 35% and about 65%, between about 35% and about 60%, between about 35% and about 55%, between about 35% and about 50%, between about 35% and about 45%, between about 40% and about 95%, between about 40% and about 90%, between about 40% and about 85%, between about 40% and about 80%, between about 40% and about 75%, between about 40% and about 70%, between about 40% and about 65%, between about 40% and about 60%, between about 40% and about 55%, between about 40% and about 50%, between about 45% and about 95%, between about 45% and about 90%, between about 45% and about 85%, between about 45% and about 80%, between about 45% and about 75%, between about 45% and about 70%, between about 45% and about 65%, between about 45% and about 60%, between about 45% and about 55%, between about 50% and about 95%, between about 50% and about 90%, between about 50% and about 85%, between about 50% and about 80%, between about 50% and about 75%, between about 50% and about 70%, between about 50% and about 65%, between about 50% and about 60%, between about 55% and about 95%, between about 55% and about 90%, between about 55% and about 85%, between about 55% and about 80%, between about 55% and about 75%, between about 55% and about 70%, between about 55% and about 65%, between about 60% and about 95%, between about 60% and about 90%, between about 60% and about 85%, between about 60% and about 80%, between about 60% and about 75%, between about 60% and about 70%, between about 65% and about 95%, between about 65% and about 90%, between about 65% and about 85%, between about 65% and about 80%, between about 65% and about 75%, between about 70% and about 95%, between about 70% and about 90%, between about 70% and about 85%, between about 70% and about 80%, between about 75% and about 95%, between about 75% and about 90%, between about 75% and about 85%, between about 80% and about 95%, between about 80% and about 90%, or between about 85% and about 95%) of the total amount of the local anesthetic agent present in the pharmaceutical composition. The percentage of the local anesthetic in its free-base form can be determined based on the pH of the solution used to dissolve the local anesthetic agent (e.g., the local anesthetic agent in its free-base form) and the encapsulation/entrapment efficiency of the drug delivery vehicle. The pH of the solution (e.g., an aqueous solution) used to dissolve the local anesthetic agent (e.g., the local anesthetic agent in its free-base form) can be determined, e.g., using a pH meter. Additional methods for determining the pH of the solution used to dissolve the local anesthetic agent (e.g., the local anesthetic agent in its free-base form) are well known in the art. The encapsulation and/or entrapment efficiency of a drug delivery vehicle (e.g., a liposome) can be determined, e.g., by the minicolumn centrifugation method (Jan Zuidam et al., Liposomes: A Practical Approach, Protocol 10, p 51, 2003) or by the protamine aggregation method (Jan Zuidam et al., Liposomes: A Practical Approach, Protocol 11, p 53, 2003). Additional methods for determining the encapsulation and/or entrapment efficiency of a drug delivery vehicle (e.g., a liposome, a microemulsion, an emulsion, a micelle, a nanoparticle, and a cyclodextrin) are well known in the art.

In some embodiments of any of the pharmaceutical compositions provided herein, the local anesthetic agent is tetracaine. In some examples, the local anesthetic agent included in any of the pharmaceutical compositions provided herein is lipophilic (e.g., tetracaine) in its free-base form. In some examples, the local anesthetic agent included in any of the pharmaceutical compositions provided herein has lipophilic properties in its base form and hydrophilic properties in its cationic form.

### Mucosal Absorption Enhancers

As is well known in the art, mucosal absorption enhancers are molecules that, when added to a pharmaceutical composition or a first pharmaceutical composition and/or second pharmaceutical composition (e.g., any of the pharmaceutical compositions, first pharmaceutical compositions, and second pharmaceutical compositions provided herein) increase the absorption (e.g., paracellular transport and/or transcellular transport) of a therapeutic agent (e.g., a local anesthetic agent) across an epithelial monolayer (e.g., a mucosal epithelium, such as the nasal mucosa) and/or increase the residence time of a therapeutic agent (e.g., a local anesthetic agent) on a targeted area of a mucosal epithelium (e.g., the nasal mucosa).

A mucosal absorption enhancer (e.g., a fatty acid, such as, e.g., sodium caprate), when added to a pharmaceutical composition (e.g., any of the pharmaceutical compositions described herein) can increase the absorption of a therapeutic agent (e.g., a local anesthetic agent in its free-base form) and/or drug delivery vehicle (e.g., any of the drug delivery vehicles described herein, e.g., a drug delivery vehicle that includes an entrapped and/or encapsulated local anesthetic agent in its free-base form) on a targeted area of a mucosal epithelium (e.g., the nasal mucosa).

The term mucosal absorption enhancer includes agents that increase transmucosal absorption by enhancing the release (e.g., from a drug delivery vehicle), solubility, diffusion rate, rate of permeation, penetration capacity, uptake, residence time, stability, peak or sustained concentration levels, and other desired mucosal delivery characteristics (e.g., as measured at the site of delivery or at a selected target site of activity) of a local anesthetic agent. Enhancement of mucosal delivery can occur by any of a variety of mechanisms, e.g. by increasing the diffusion, transport, or stability of an anesthetic agent, increasing membrane fluidity, modulating the availability or action of calcium and other ions that regulate transcellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms. A mucosal absorption enhancer can be administered to a subject in one administration (e.g., in a first pharmaceutical composition or in a second pharmaceutical composition) and the local anesthetic agent can be administered, e.g., at the same time or a substantially the same time, in a separate administration (e.g., in a first pharmaceutical composition or a second pharmaceutical composition), and the mucosal absorption enhancer results in an increase in the absorption (e.g., paracellular transport and/or transcellular transport) of the local anesthetic agent across an epithelial monolayer (e.g., a mucosal epithelium, such as the nasal mucosa) and/or an increase in the residence time of the local anesthetic agent on a targeted area of a mucosal epithelium (e.g., the nasal mucosa).

Mucosal absorption enhancers are used to increase the absorption (e.g., paracellular transport and/or transcellular transport) of poorly absorbed drugs (e.g., small hydrophilic drugs), but not used to increase absorption (e.g., paracellular transport and/or transcellular transport) of local anesthetic agents, as these local anesthetic agents are known to readily pass through a mucosal epithelium (e.g., the nasal mucosa). Without being bound by any theory, the use of mucosal absorption enhancers in the present methods is thought to increase the absorption of the cation species of the local anesthetic agent across the nasal mucosa when the local anesthetic agent is administered as a salt. This is thought to thereby increase the quantity of drug that is able to travel along the trigeminal nerve pathway and be absorbed into more distant nerve tissue (e.g., nerve tissue of the trigeminal nerve pathway), e.g., after experiencing a substantial shift in pH from the acidic nasal mucosa to more typical physiological pH values.

Non-limiting examples of mucosal absorption enhancers that can be included in any of the pharmaceutical compositions provided herein or any of the first pharmaceutical compositions and/or second pharmaceutical compositions provided herein include: monosaccharides (e.g., glucose, galactose, mannose, 3-O-methyl glucose, xylose, ribose, arabinose, ribulose, fructose, and sorbose) (e.g., in either the D- or L-form), polysaccharides (e.g., linear polysaccharides, e.g., chitosan or trimethyl chitosan, such as chitosan hydrochloride and chitosan glutamate), a bile salt (e.g., cholate, sodium taurocholate, sodium glycocholate, sodium deoxycholate, and sodium glycodeoxycholate), a surfactant (e.g., sodium lauryl sulfate, polyoxyethyleneglycol dodecyl ether, polyoxyethylene 10 oleoyl ether, lysophosphatidylcholine, dioctyl sodium sulfosuccinate, polyoxyethylene 9 lauryl ether, polysorbate 80, polyethyleneglycol-8-laurate, and glyceryl monolaurate), an oil (see, e.g., Chen et al., Asian J. Pharmacol. 9: 51-64, 2014), a fusidate compound (e.g., sodium fusidate or sodium dihydrotaurofusidate), a cyclodextrin (e.g., cyclodextrin, beta-cyclodextrin, hydroxypropyl beta-cyclodextrin, or dimethyl-beta-cyclodextrin), a phospholipid (e.g., didecanoyl-L-mu-phosphatidylcholine, lysophosphatidylcholine, and didecanoyl-L-phosphatidylcholine), a thiomer (e.g., chitosan-cysteine, chitosan-thiogylcolic acid, chitosan-thiobutylamidine, deacetylated gellan gum-cysteine, poly(methacrylic acid)-cysteine, alginate-cysteine, carboxymethycellulose-cysteine, poly(acrylic acid)-cysteine, poly(acrylic acid)-cysteamine, poly(acrylic acid)-homocysteine, and thiolated polycarbophil), a fatty acid (e.g., a glyceride, such as a monoglyceride, a diglyceride, or a triglyceride, a monohexanoin, a medium chain glyceride, sodium caprate, sodium lauryl sulfate, sorbitan laurate, sucrose palmitate, lauroyl choline, sodium myristate, palmitoyl carnitine, and their derivatives), a chelator (e.g., ethylene diamine tetraacetate or disodium ethylene diamine tetraacetate), a salicylate (e.g., salicylic acid, sodium methoxysalicylate, or acetylsalicylic acid), a polymer (e.g., polycarbonyl, sodium carboxymethylcellulose, and their derivatives), a micelle (see, e.g., Jhaveri et al., Front. Pharmacol. 5:77, 2014), an alcohol (e.g., isopropyl alcohol), a liposome (see, e.g., Eloy et al., Colloids Surf. B Biointerfaces 123C: 345-363, 2014; van der Meel et al., J. Control Release 195C: 72-85, 2014), a microemulsion (see, e.g., Yu et al., J. Pharm. Sci. 100: 933-941, 2011), an emulsion (see, e.g., Du et al., Biomacromolecules 15:1097-1114, 2014; Russell-Jones et al., Expert. Opin. Drug Deliv. 8:537-546, 2011; He et al., Expert. Opin. Drug Deliv. 7:445-460, 2010; Mehta et al., Recent Pat. Drug Deliv. Formul. 4:35-48, 2010), a solid lipid nanoparticle (see, e.g., Jose et al., Int. J. Pharm. 474: 6-13, 2014) a transferosome (see, e.g., Reshmy et al. J. Adv. Pharm. Technol. Res. 2:138-143, 2011), propylene glycol, menthol, ammonium glycyyhizinate, glycrrhetinic acid, aminated gelatin, laurocapram, benzalkonium chloride, a phenothioate, a nitric acid donor, zonula occluden toxin, a poly-L-arginine, a soybean derivative glucoside (see, e.g., Maitani, Yakugaku Zasshi 116:901-910, 1996), citicholine, and an alpha-acid derivative. Additional examples of mucosal absorption enhancers are known in the art.

Drug delivery vehicles can act as mucosal absorption enhancers in the intranasal delivery of local anesthetic agents. Drug delivery vehicles are carrier systems that can be included in pharmaceutical compositions in order to, e.g., improve drug action and safety, often by improving in vivo biodistribution of the drug including, e.g., transmucosal absorption. Non-limiting examples of drug delivery vehicles include liposomes, microemulsions, emulsions, micelles, lipid nanoparticles, and cyclodextrins. Some embodiments of any of the pharmaceutical compositions described herein can include a drug delivery vehicle (e.g., any of the drug delivery vehicles described herein or known in the art).

In some embodiments of any of the pharmaceutical compositions described herein, the local anesthetic agent (e.g., the base form of the local anesthetic agent) is entrapped and/or encapsulated by the drug delivery vehicle. In some embodiments of any of the pharmaceutical compositions described herein, the local anesthetic agent (e.g., the free-base form of the local anesthetic agent) is formulated in the drug delivery vehicle.

A liposome is a vesicular lipid bilayer that encloses an aqueous region. Liposomes can include one or more of the following: a phospholipid, an excipient, and a local anesthetic agent (e.g., any of the exemplary local anesthetics provided herein). A liposome can encapsulate a local anesthetic agent (e.g., a local anesthetic agent in its ionized form) within the aqueous region of the liposome and/or can entrap the local anesthetic agent (e.g., a local anesthetic agent in its free-base form) within the lipid bilayer of the liposome. For example, liposomes are capable of carrying (e.g., entrapping and/or encapsulating), e.g., lipophilic drugs within their lipid bilayer and hydrophilic drugs within their aqueous inner region. Any of the liposomes in any of the pharmaceutical compositions described herein can have a net charge that is cationic, neutral, or anionic.

The lipid bilayer of a liposome can be composed, at least in part, of one or more phospholipids (e.g., one or more synthetic phospholipids and/or naturally-occurring phospholipids) that can have a net charge (e.g., a net positive or net negative charge) or can be neutral in charge. Non-limiting examples of a phospholipid that can be included in any of the pharmaceutical compositions herein include phosphatidylcholines, lysophosphatidylcholines, phosphatidylserines, phosphatidylethanolamines, phosphatidylglycerols, and phosphatidylinositols. As can be appreciated by those skilled in the art, one or more of any of the exemplary phospholipids described herein or known in the art can be present in the lipid bilayer of a liposome.

Liposomes can also, e.g., contain an excipient (e.g., cholesterol) within the lipid bilayer in order to alter their characteristics (e.g., increase stability and/or increase drug encapsulation and/or entrapment efficiency) and/or, e.g., be coated (e.g., surface modification) with an excipient (e.g., a polymer, such as, e.g., a chitosan)) in order to alter their characteristics (e.g., increase mucoadhesion). Non-limiting examples of an excipient that can be included in any of the pharmaceutical compositions herein include a cholesterol, a stearylamine, a stearic acid, a tocopherol, a polymer, a chitosan, and a polyacrylic acid. In some examples, a liposome can include a phospholipid and an excipient (e.g., and an entrapped and/or encapsulated local anesthetic agent in its free-base form).

Liposomes can have an average diameter of between about 2.5 nm and about 3,000 nm (e.g., between 2.5 nm and about 2,500 nm, between about 2.5 nm and about 2,000 nm, between about 2.5 nm and about 1,800 nm, between about 2.5 nm and about 1,600 nm, between about 2.5 nm and about 1,400 nm, between about 2.5 nm and about 1.200 nm, between about 2.5 nm and about 1,000 nm, between about 2.5 nm and about 800 nm, between about 2.5 nm and about 600 nm, between about 2.5 nm and about 400 nm, between about 2.5 nm and about 200 nm, between about 2.5 nm and about 100 nm, between about 2.5 nm and about 50 nm, between about 5 nm and about 3,000 nm, between about 5 nm and about 2,500 nm, between about 5 nm and about 2,000 nm, between about 5 nm and about 1,800 nm, between about 5 nm and about 1,600 nm, between about 5 nm and about 1,400 nm, between about 5 nm and about 1,200 nm, between about 5 nm and about 1,000 nm, between about 5 nm and about 800 nm, between about 5 nm and about 600 nm, between about 5 nm and about 400 nm, between about 5 nm and about 200 nm, between about 5 nm and about 100 nm, between about 5 nm and about 50 nm, between about 10 nm and about 3,000 nm, between about 10 nm and about 2,500 nm, between about 10 nm and about 2,000 nm, between about 10 nm and about 1,500 nm, between about 10 nm and about 1,000 nm, between about 10 nm and about 800 nm, between about 10 nm and about 600 nm, between about 10 nm and about 400 nm, between about 10 nm and about 200 nm, between about 10 nm and about 100 nm, between about 10 nm and about 50 nm, between about 25 nm and about 3,000 nm, between about 25 nm and about 2,500 nm, between about 25 nm and about 2,000 nm, between about 25 nm and about 1,500 nm, between about 25 nm and about 1,000 nm, between about 25 nm and about 800 nm, between about 25 nm and about 600 nm, between about 25 nm and about 400 nm, between about 25 nm and about 200 nm, between about 25 nm and about 100 nm, between about 50 nm and about 3,000 nm, between about 50 nm and about 2,500 nm, between about 50 nm and about 2,000 nm, between about 50 nm and about 1,500 nm, between about 50 nm and about 1,000 nm, between about 50 nm and about 800 nm, between about 50 nm and about 600 nm, between about 50 nm and about 400 nm, between about 50 nm and about 200 nm, between about 50 nm and about 100 nm, between about 100 nm and about 3,000 nm, between about 100 nm and about 2,500 nm, between about 100 nm and about 2,000 nm, between about 100 nm and about 1,500 nm, between about 100 nm and about 1,000 nm, between about 100 nm and about 800 nm, between about 100 nm and about 600 nm, between about 100 nm and about 400 nm, between about 100 nm and about 200 nm, between about 150 nm and about 3,000 nm, between about 150 nm and about 2,500 nm, between about 150 nm and about 2,000 nm, between about 150 nm and about 1,500 nm, between about 150 nm and about 1,000 nm, between about 150 nm and about 800 nm, between about 150 nm and about 600 nm, between about 150 nm and about 400 nm, between about 150 nm and about 200 nm, between about 200 nm and about 3,000 nm, between about 200 nm and about 2,500 nm, between about 200 nm and about 2,000 nm, between about 200 nm and about 1,500 nm, between about 200 nm and about 1,000 nm, between about 200 nm and about 800 nm, between about 200 nm and about 600 nm, between about 200 nm and about 400 nm, between about 250 nm and about 3,000 nm, between about 250 nm and about 2,500 nm, between about 250 nm and about 2,000 nm, between about 250 nm and about 1,500 nm, between about 250 nm and about 1,000 nm, between about 250 nm and about 800 nm, between about 250 nm and about 600 nm, between about 250 nm and about 400 nm, between about 500 nm and about 3,000 nm, between about 500 nm and about 2,500 nm, between about 500 nm and about 2,000 nm, between about 500 nm and about 1,500 nm, between about 500 nm and about 1,000 nm, between about 500 nm and about 800 nm, between about 500 nm and about 600 nm, between about 1,000 nm and about 3,000 nm, between about 1,000 nm and about 2,500 nm, between about 1,000 nm and about 2,000 nm, between about 1,000 nm and about 1,500 nm, between about 1,500 nm and about 3,000 nm, between about 1,500 nm and about 2,500 nm, between about 1,500 nm and about 2,000 nm, between about 2,000 nm and about 3,000 nm, between about 2,000 nm and about 2,500 nm, or between about 2,500 nm and about 3,000 nm).

Liposomes are categorized by the number of lamellae (e.g., multiple lamellae) they have and by their size (e.g., an average diameter of 200 nm). Non-limiting examples of a liposome that can be included in any of the pharmaceutical compositions herein include: a small unilamellar vesicle, a large unilamellar vesicle, a giant unilamellar vesicle, a multilamellar vesicle, and a multivesicular vesicle. Exemplary features of small unilamellar vesicles (see, e.g., Decker et al., J. Liposome Res. 23:154-165, 2013; Lin et al., Langmuir 28:689-700, 2012; and Lohse et al., J. Am. Chem. Soc. 130:14372-14373, 2008), large unilamellar vesicles (see, e.g., Allen et al., FEBS Lett 223:42-46, 1987; U.S. Pat. No. 4,078,052; Hope et al., Biochim. Biophys. Acta Biomembranes 812:55-65, 1985; and U.S. Patent Application Publication No. 2008/0241233), giant unilamellar vesicles (see, e.g., Moscho et al., Proc. Natl. Acad. Sci. U.S.A. 93:11443-11447, 1996; Pott et al., Chem. Phys. Lipids 154:115-119, 2008; and Carvalho et al., Biophys. 1 95:4348-4360, 2008), multilamellar vesicles (see, e.g., Moon et al., Nat. Mater. 10:243-251, 2011; Hope et al., Chem. Phys. Lipids 40:89-107, 1986; Vyas et al., Pharm. Acta Helv. 74:51-58, 1999; and Kim et al., Biochim. Biophys. Acta Biomembranes 812:793-801, 1985), and multivesicular vesicles (see, e.g., Cohen et al., J. Materials Chem. B 1:4619-4627, 2013; U.S. Pat. No. 5,993,850; Kim et al., Biochim. Biophys. Acta Biomembranes 728:339-348, 1983; and Qiu et al., Acta Pharmacologica Sinica 26:1395-1401, 2005) are known in the art. For example, small unilamellar vesicles have one lamellae and typically have an average diameter of about 20 to about 100 nm (e.g., between about 20 nm to about 90 nm, between about 20 nm to about 80 nm, between about 20 nm to about 70 nm, between about 20 nm to about 60 nm, between about 20 nm to about 50 nm, between about 20 nm to about 40 nm, between about 20 nm to about 30 nm, between about 25 nm to about 90 nm, between about 25 nm to about 80 nm, between about 25 nm to about 70 nm, between about 25 nm to about 60 nm, between about 25 nm to about 50 nm, between about 25 nm to about 40 nm, between about 25 nm to about 35 nm, between about 30 nm to about 90 nm, between about 30 nm to about 80 nm, between about 30 nm to about 70 nm, between about 30 nm to about 60 nm, between about 30 nm to about 50 nm, between about 30 nm to about 40 nm, between about 35 nm to about 90 nm, between about 35 nm to about 80 nm, between about 35 nm to about 70 nm, between about 35 nm to about 60 nm, between about 35 nm to about 50 nm, between about 35 nm to about 45 nm, between about 40 nm to about 90 nm, between about 40 nm to about 80 nm, between about 40 nm to about 70 nm, between about 40 nm to about 60 nm, between about 40 nm to about 50 nm, between about 50 nm to about 90 nm, between about 50 nm to about 80 nm, between about 50 nm to about 70 nm, between about 50 nm to about 60 nm, between about 60 nm to about 90 nm, between about 60 nm to about 80 nm, between about 60 nm to about 70 nm, between about 70 nm to about 90 nm, between about 70 nm to about 80 nm, or between about 80 nm to about 90 nm). Large unilamellar vesicles have one lamellae and typically have an average diameter of between about 100 nm to about 400 nm (e.g., between about 100 nm to about 350 nm, between about 100 nm to about 300 nm, between about 100 nm to about 250 nm, between about 100 nm to about 200 nm, between about 100 nm to about 150 nm, between about 150 nm to about 400 nm, between about 150 nm to about 350 nm, between about 150 nm to about 300 nm, between about 150 nm to about 250 nm, between about 150 nm to about 200 nm, between about 200 nm to about 400 nm, between about 200 nm to about 350 nm, between about 200 nm to about 300 nm, between about 200 nm to about 250 nm, between about 250 nm to about 400 nm, between about 250 nm to about 350 nm, between about 250 nm to about 300 nm, between about 300 nm to about 400 nm, between about 300 nm to about 350 nm, or between 350 nm to about 400 nm). Giant unilamellar vesicles have one lamellae and typically have an average diameter of greater than about 1,000 nm (e.g., between about 1,000 nm and about 3,000 nm, between about 1,000 nm to about 2,500 nm, between about 1,000 nm to about 2,000 nm, between about 1,000 nm to about 1,800 nm, between about 1,000 nm to about 1,600 nm, between about 1,000 nm to about 1,400 nm, between about 1,000 nm to about 1,200 nm, between about 1,500 nm to about 3,000 nm, between about 1,500 nm to about 2,500 nm, between about 1,500 nm to about 2,000 nm, between about 2,000 nm to about 3,000 nm, between about 2,000 nm to about 2,500 nm, or between about 2,500 nm to about 3,000 nm). Multilamellar vesicles have multiple lamellae and typically have an average diameter of between about 200 nm to about 3,000 nm (e.g., between about 200 nm to about 2,500 nm, between about 200 nm to about 2,000 nm, between about 200 nm to about 1,500 nm, between about 200 nm to about 1,000 nm, between about 200 nm to about 800 nm, between about 200 nm to about 600 nm, between about 200 nm to about 400 nm, between about 500 nm to about 3,000 nm, between about 500 nm to about 2,500 nm, between about 500 nm to about 2,000 nm, between about 500 nm to about 1,500 nm, between about 500 nm to about 1,000 nm, between about 1,000 nm to about 3,000 nm, between about 1,000 nm to about 2,500 nm, between about 1,000 nm to about 2,000 nm, between about 1,000 nm to about 1,500 nm, between about 1,500 nm to about 3,000 nm, between about 1,500 nm to about 2,500 nm, between about 1,500 nm to about 2,000 nm, between about 2,000 nm to about 3,000 nm, or between about 2,500 nm to about 3,000 nm). Additional aspects of liposomes that can be used for entrapping and encapsulating a drug and methods of making liposomes are described in, e.g., Akbarzadeh et al., Nanoscale Res. Lett. 8:102, 2013; Anarnath et al., Int. J. Pharm. 154:123-140, 1997; Bozzuto et al., Int. J. Nanomedicine 10:975-999, 2015; van der Meel et al., J. Control Release 195:72-85, 2014; Mittal et al., J. Drug Target. 22:372-386, 2014; Kroon et al., Cancer Treat Rev. 40:578-584, 2014; Karn et al., Nanomedicine 8:1529-1548, 2013; Arias, Expert Opin. Ther. Pat. 23:1399-1414, 2013; and Eloy et al., Colloids Surf. B Biointerfaces 123C:345-363, 2014.

Microemulsions are art known and are generally systems including water, lipid, and surfactant that can, e.g., be utilized to carry drugs either in the lipid phase or aqueous phase. Microemulsions can, e.g., optionally include a co-surfactant. The surfactant, and optionally the co-surfactant, in the microemulsion stabilize the dispersed phase. Microemulsions are thermodynamically stable, colloidal mixtures that can also be, e.g., clear in solution. A microemulsion can be classified by its continuous phase and dispersed phase. For example, a microemulsion can be oil-in-water (o/w), water-in-oil (w/o), or bi-continuous.

The concentration of the aqueous phase, lipid phase, and surfactant(s) in an oil-in-water (o/w) microemulsion in any of the pharmaceutical compositions provided herein can be determined by taking into account the specific lipid and surfactant(s) that are included in the composition, as well as the desired strength and activity of the pharmaceutical composition, and the specific local anesthetic agent(s) included in the pharmaceutical composition. The aqueous phase of an oil-in-water (o/w) microemulsion can be present in the pharmaceutical composition (e.g., a liquid, a gel, or paste) at a concentration of at least about 10% (e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%) by weight of the pharmaceutical composition (e.g., w/w or w/v). The lipid phase of an oil-in-water (o/w) microemulsion can be present in the pharmaceutical composition (e.g., a liquid, a gel, or paste) at a concentration of between about 0.1% to about 50% (e.g., between about 1% to about 50%, between about 1% to about 40%, between about 1% to about 30%, between about 1% to about 20%, between about 1% to about 10%, between about 1% to about 5%, between about 5% to about 50%, between about 5% to about 40%, between about 5% to about 35%, between about 5% to about 30%, between about 5% to about 25%, between about 5% to about 20%, between about 5% to about 15%, between about 5% to about 10%, between about 10% to about 50%, between about 10% to about 40%, between about 10% to about 30%, between about 10% to about 25%, between about 10% to about 20%, between about 10% to about 15%, between about 20% to about 50%, between about 20% to about 45%, between about 20% to about 40%, between about 20% to about 35%, between about 20% to about 30%, between about 20% to about 35%, between about 25% to about 50%, between about 25% to about 45%, between about 25% to about 40%, between about 25% to about 30%, between about 30% to about 45%, between about 30% to about 40%, between about 30% to about 35%, between about 40% to about 50%, or between about 40% to about 45%) by weight of the pharmaceutical composition (e.g., w/w or w/v). The surfactant(s) of an oil-in-water (o/w) microemulsion can be present in the pharmaceutical composition (e.g., a liquid, a gel, or paste) at a concentration of between about 0.1% to about 80% (e.g., between about 0.1% to about 80%, between about 0.1% to about 75%, between about 0.1% to about 70%, between about 0.1% to about 65%, between about 0.1% to about 60%, between about 0.1% to about 55%, between about 0.1% to about 50%, between about 0.1% to about 45%, between about 0.1% to about 40%, between about 0.1% to about 35%, between about 0.1% to about 30%, between about 0.1% to about 25%, between about 0.1% to about 20%, between about 0.1% to about 15%, between about 0.1% to about 10%, between about 0.1% to about 8%, between about 0.1% to about 5%, between about 0.1% to about 3%, between about 1% to about 80%, between about 1% to about 70%, between about 1% to about 60%, between about 1% to about 50%, between about 1% to about 40%, between about 1% to about 30%, between about 1% to about 20%, between about 1% to about 10%, between about 1% to about 5%, between about 10% to about 50%, between about 10% to about 45%, between about 10% to about 40%, between about 10% to about 35%, between about 10% to about 30%, between about 10% to about 20%, between about 10% to about 15%, between about 15% to about 50%, between about 15% to about 40%, between about 15% to about 30%, between about 15% to about 25%, between about 18% to about 25%, between about 18% to about 22%, or between about 19% to about 22%) by weight of the pharmaceutical composition (e.g., w/w or w/v).

Non-limiting examples of the lipid to total surfactant (e.g., the surfactant and the co-surfactant) ratio that can be included in any of the pharmaceutical compositions herein include about 1:1 to about 1:10. Non-limiting examples of the surfactant to co-surfactant ratio that can be included in any of the pharmaceutical compositions herein include about 6:1 to about 1:6. Non-limiting examples of the surfactant to co-surfactant ratio that can be included in any of the pharmaceutical compositions herein include about 3:1 to about 1:3.

Non-limiting examples of the lipid that can be included in any of the pharmaceutical compositions herein include castor oil, corn oil, cottonseed oil, olive oil, peanut oil, soybean oil, vegetable oil, coconut oil, isopropyl palmitate, light mineral oil, oleic acid, medium chain mono/diglycerides, and propylene glycol dicaprylocaprate. Non-limiting examples of the lipid that can be included in any of the pharmaceutical compositions herein include one or more local anesthetics in oil form. Non-limiting examples of a surfactant and a co-surfactant that can be included in any of the pharmaceutical compositions herein include polysorbate 20, polysorbate 40, polysorbate 60, polysorbate 65, polysorbate 80, polyoxyl 8 stearate, polypropylene glycol, propylene glycol, carbomer 1342, carbomer 934, carbomer 934P, carbomer 940, carbomer 941, carbomer 974, carbomer 980, carbomer 981, oleyl polyethylene glycol glyceride, PEG-300, PEG-400, PEG-3500, poloxamer 124, poloxamer 188, poloxamer 407, polyoxyl 35 castor oil, polyoxyl 40 hydrogenated castor oil, polyoxyl 40 stearate, polyoxyl 6 and polyoxyl 32 palmitostearate, polyoxyl 8 stearate, polyoxyl glyceryl stearate, polyoxyl lanolin, polyoxyl palmitate, polyoxyl stearate, glyceryl oleate, propylene carbonate, sorbitan monooleate, glycerin, PEG-5 oleate, sodium N-methyl N-cocoyl taurate, PEG-8 caprylic/capric gylcerides, and diethylene glycol monoethyl ether.

A microemulsion can include droplets having an average diameter of between, e.g., about 1 nm to about 200 nm (e.g., between about 1 nm to about 195 nm, between about 1 nm to about 190 nm, between about 1 nm to about 185 nm, between about 1 nm to about 180 nm, between about 1 nm to about 175 nm, between about 1 nm to about 170 nm, between about 1 nm to about 165 nm, between about 1 nm to about 160 nm, between about 1 nm to about 155 nm, between about 1 nm to about 150 nm, between about 1 nm to about 145 nm, between about 1 nm to about 140 nm, between about 1 nm to about 135 nm, between about 1 nm to about 130 nm, between about 1 nm to about 125 nm, between about 1 nm to about 120 nm, between about 1 nm to about 115 nm, between about 1 nm to about 110 nm, between about 1 nm to about 105 nm, between about 5 nm to about 100 nm, between about 5 nm to about 95 nm, between about 5 nm to about 90 nm, between about 5 nm to about 85 nm, between about 5 nm to about 80 nm, between about 5 nm to about 75 nm, between about 5 nm to about 70 nm, between about 5 nm to about 65 nm, between about 5 nm to about 60 nm, between about 5 nm to about 55 nm, between about 5 nm to about 50 nm, between about 5 nm to about 45 nm, between about 5 nm to about 40 nm, between about 5 nm to about 35 nm, between about 5 nm to about 30 nm, between about 5 nm to about 25 nm, between about 5 nm to about 20 nm, between about 5 nm to about 15 nm, between about 5 nm to about 10 nm, between about 10 nm to about 200 nm, between about 10 nm to about 195 nm, between about 10 nm to about 190 nm, between about 10 nm to about 185 nm, between about 10 nm to about 180 nm, between about 10 nm to about 175 nm, between about 10 nm to about 170 nm, between about 10 nm to about 165 nm, between about 10 nm to about 160 nm, between about 10 nm to about 155 nm, between about 10 nm to about 150 nm, between about 10 nm to about 145 nm, between about 10 nm to about 140 nm, between about 10 nm to about 135 nm, between about 10 nm to about 130 nm, between about 10 nm to about 125 nm, between about 10 nm to about 120 nm, between about 10 nm to about 115 nm, between about 10 nm to about 110 nm, between about 10 nm to about 100 nm, between about 15 nm to about 100 nm, between about 15 nm to about 90 nm, between about 15 nm to about 80 nm, between about 15 nm to about 75 nm, between about 15 nm to about 70 nm, between about 15 nm to about 65 nm, between about 15 nm to about 60 nm, between about 15 nm to about 55 nm, between about 15 nm to about 50 nm, between about 15 nm to about 45 nm, between about 15 nm to about 40 nm, between about 15 nm to about 35 nm, between about 15 nm to about 30 nm, between about 15 nm to about 25 nm, between about 15 nm to about 20 nm, between about 20 nm to about 100 nm, between about 20 nm to about 95 nm, between about 20 nm to about 90 nm, between about 20 nm to about 85 nm, between about 20 nm to about 80 nm, between about 20 nm to about 75 nm, between about 20 nm to about 70 nm, between about 20 nm to about 65 nm, between about 20 nm to about 60 nm, between about 20 nm to about 55 nm, between about 20 nm to about 50 nm, between about 20 nm to about 45 nm, between about 20 nm to about 40 nm, between about 20 nm to about 35 nm, between about 20 nm to about 30 nm, between about 20 nm to about 25 nm, between about 25 nm to about 100 nm, between about 25 nm to about 95 nm, between about 25 nm to about 90 nm, between about 25 nm to about 85 nm, between about 25 nm to about 80 nm, between about 25 nm to about 75 nm, between about 25 nm to about 70 nm, between about 25 nm to about 65 nm, between about 25 nm to about 60 nm, between about 25 nm to about 55 nm, between about 25 nm to about 50 nm, between about 25 nm to about 45 nm, between about 25 nm to about 40 nm, between about 25 nm to about 35 nm, between about 25 nm to about 30 nm, between about 50 nm to about 100 nm, between about 50 nm to about 90 nm, between about 50 nm to about 80 nm, between about 50 nm to about 70 nm, between about 50 nm to about 60 nm, between about 75 nm to about 100 nm, between about 75 nm to about 90 nm, or between about 75 nm to about 85 nm). A local anesthetic agent (e.g., any of the exemplary local anesthetic agents provided herein) can be entrapped by one or more droplets present in a microemulsion. Additional aspects of microemulsions that can be used for entrapping and/or encapsulating a drug and methods of making microemlusions are described in, e.g., Panapisal et al., AAPS PharmaSciTech 13:389-399, 2012; Baboota et al., PDA J. Pharm. Sci. Technol. 61:276-285, 2007; Mandal et al., Int. J. Pharm. Sci. Nanotechnol. 3:1214-1219, 2011; Sharma et al., J. Pharm Drug. Deliv. Res. 1:3, 2012; Todosijevic et al., Colloid Polymer Sci. 292:3061-3076, 2014; Patel et al., Int. J. Biomed. Pharm. Sci. 7:20-27, 2013; Abdelrahim et al., Int. J. Nov. Drug Deliv. Tech. 1:208-212, 2011; and Yu et al., J. Pharm. Sci. 100: 933-941, 2011. Additional non-limiting examples of the properties and composition of microemulsions are described herein.

Emulsions are art known and are a dispersion of droplets of one liquid in another liquid in which it is normally immiscible. An emulsion is a colloid. An emulsion can, e.g., entrap a local anesthetic agent (e.g., any of the exemplary local anesthetic agents provided herein) in one or more of its droplets. The droplets in an emulsion can have an average diameter of between about 10 nm to about 100 μm (e.g., between about 10 nm to about 50 μm, between about 10 nm to about 25 μm, between about 10 nm to about 10 μm, between about 10 nm to about 5 μm, between about 10 nm to about 1 μm, between about 10 nm to about 800 nm, between about 10 nm to about 600 nm, between about 10 nm to about 400 nm, between about 10 nm to about 200 nm, between about 10 nm to about 150 nm, between about 10 nm to about 100 nm, between about 10 nm to about 50 nm, between about 25 nm to about 100 μm, between 25 nm to about 50 μm, between about 25 nm to about 25 μm, between about 25 nm to about 10 μm, between about 25 nm to about 5 μm, between about 25 nm to about 1 μm, between about 25 nm to about 800 nm, between about 25 nm to about 600 nm, between about 25 nm to about 400 nm, between about 25 nm to about 200 nm, between about 25 nm to about 150 nm, between about 25 nm to about 100 nm, between about 25 nm to about 50 nm, between about 50 nm to about 100 μm, between about 50 nm to about 50 μm, between about 50 nm to about 25 μm, between about 50 nm to about 5 μm, between about 50 nm to about 1 μm, between about 50 nm to about 800 nm, between about 50 nm to about 600 nm, between about 50 nm to about 400 nm, between about 50 nm to about 200 nm, between about 50 nm to about 150 nm, between about 50 nm to about 100 nm, between about 100 nm to about 100 μm, between about 100 nm to about 50 μm, between about 100 nm to about 25 μm, between about 100 nm to about 10 μm, between about 100 nm to about 5 μm, between about 100 nm to about 1 μm, between about 100 nm to about 800 nm, between about 100 nm to about 600 nm, between about 100 nm to about 400 nm, between about 100 nm to about 200 nm, between about 100 nm to about 150 nm, between about 150 nm to about 100 μm, between about 150 nm to about 50 μm, between about 150 nm to about 25 μm, between about 150 nm to about 10 μm, between about 150 nm to about 5 μm, between about 150 nm to about 1 μm, between about 150 nm to about 800 nm, between about 150 nm to about 600 nm, between about 150 nm to about 400 nm, between about 150 nm to about 200 nm, between about 200 nm to about 100 μm, between about 200 nm to about 50 μm, between about 200 nm to about 25 μm, between about 200 nm to about 10 μtm, between about 200 nm to about 5 μm, between about 200 nm to about 1 μm, between about 200 nm to about 800 nm, between about 200 nm to about 600 nm, between about 200 nm to about 400 nm, between about 250 nm to about 100 μm, between about 250 nm to about 50 μm, between about 250 nm to about 25 μm, between about 250 nm to about 10 μm, between about 250 nm to about 5 μm, between about 250 nm to about 1 μm, between about 250 nm to about 800 nm, between about 250 nm to about 600 nm, between about 250 nm to about 400 nm, between about 500 nm to about 100 μm, between about 500 nm to about 50 μm, between about 500 nm to about 25 μm, between about 500 nm to 10 μm, between about 500 nm to about 5 μm, between about 500 nm to about 1 μm, between about 500 nm to about 800 nm, between about 1 μm to about 100 μm, between about 1 μm to about 50 μm, between about 1 μm to about 25 μm, between about 1 μm to about 10 μm, between about 1 μm to about 5 μm, between about 5 μm to about 100 μm, between about 5 μm to about 50 μm, between about 5 μm to about 25 μm, between about 5 μm to about 10 μm, between about 10 μm to about 100 μm, between about 10 μm to about 50 μm, between about 10 μm to about 25 μm, between about 25 μm to about 100 μm, between about 25 μm to about 50 μm, or between about 50 μm to about 100 μm). Additional aspects of emulsions that can be used for entrapping and/or encapsulating a drug and methods of making emulsions are described in, e.g., Collins-Gold et al., Adv. Drug Delivery Rev. 5:189-208, 1990; Washington, Adv. Drug Delivery Rev. 20:131-145, 1996; Davis et al., Ann. NY Acad Sci. 507:75-88, 1987; and Wadhwa et al., Acta Poloniae Pharm. 69:179-191, 2012.

Micelles are art known and include a lipid monolayer enclosing a hydrophobic region. A micelle can, e.g., encapsulate a local anesthetic agent (e.g., any of the exemplary local anesthetic agents provided herein) in its enclosed hydrophobic region and/or can entrap a local anesthetic agent (e.g., any of the exemplary local anesthetic agents provided herein) in its lipid monolayer. Micelles can include amphiphilic molecules, e.g., amphiphilic di- or tri-block copolymers. Non-limiting aspects of micelles are described herein. Micelles can have an average diameter of, e.g., between about 2.0 nm to about 30 nm (e.g., between about 2.0 nm to about 28 nm, between about 2.0 nm to about 26 nm, between about 2.0 nm to about 24 nm, between about 2.0 nm to about 24 nm, between about 2.0 nm to about 22 nm, between about 2.0 nm to about 20 nm, between about 2.0 nm to about 18 nm, between about 2.0 nm to about 16 nm, between about 2.0 nm to about 14 nm, between about 2.0 nm to about 12 nm, between about 2.0 nm to about 10 nm, between about 2.0 nm to about 8.0 nm, between about 2.0 nm to about 6.0 nm, between about 2.0 nm to about 4.0 nm, between about 4.0 nm to about 30 nm, between about 4.0 nm to about 28 nm, between about 4.0 nm to about 26 nm, between about 4.0 nm to about 24 nm, between about 4.0 nm to about 22 nm, between about 4.0 nm to about 20 nm, between about 4.0 nm to about 18 nm, between about 4.0 nm to about 16 nm, between about 4.0 nm to about 14 nm, between about 4.0 nm to about 12 nm, between about 4.0 nm to about 10 nm, between about 4.0 nm to about 8.0 nm, between about 4.0 nm to about 6.0 nm, between about 6.0 nm to about 30 nm, between about 6.0 nm to about 28 nm, between about 6.0 nm to about 26 nm, between about 6.0 nm to about 24 nm, between about 6.0 nm to about 22 nm, between about 6.0 nm to about 20 nm, between about 6.0 nm to about 18 nm, between about 6.0 nm to about 16 nm, between about 6.0 nm to about 14 nm, between about 6.0 nm to about 12 nm, between about 6.0 nm to about 10 nm, between about 6.0 nm to about 8.0 nm, between about 8.0 nm to about 30 nm, between about 8.0 nm to about 28 nm, between about 8.0 nm to about 26 nm, between about 8.0 nm to about 24 nm, between about 8.0 nm to about 22 nm, between about 8.0 nm to about 20 nm, between about 8.0 nm to about 18 nm, between about 8.0 nm to about 16 nm, between about 8.0 nm to about 14 nm, between about 8.0 nm to about 12 nm, between about 8.0 nm to about 10 nm, between about 10 nm to about 30 nm, between about 10 nm to about 28 nm, between about 10 nm to about 26 nm, between about 10 nm to about 24 nm, between about 10 nm to about 22 nm, between about 10 nm to about 20 nm, between about 10 nm to about 18 nm, between about 10 nm to about 16 nm, between about 10 nm to about 14 nm, between about 10 nm to about 12 nm, between about 15 nm to about 30 nm, between about 15 nm to about 25 nm, between about 15 nm to about 20 nm, between about 20 nm to about 30 nm, between about 20 nm to about 25 nm, or between about 25 nm to about 30 nm). Additional aspects of micelles that can be used for entrapping and/or encapsulating a drug and methods of making micelles are described in, e.g., Wang et al., Int. J. Nanomed. 7:4487-4497, 2012; Bachhav et al., J. Controlled Release 153:126-132, 2011; WO 03/047493; U.S. Patent Application

Publication No. 2009/0036389; U.S. Patent Application Publication No. 2008/0248097; and Liu et al., Curr. Pharmaceutical Design 12(36):4685-4701, 2006.

Lipid nanoparticles (also known as solid lipid nanoparticles) are art known and are particles that include a solid lipid core matrix that is stabilized by a surfactant. A lipid nanoparticle can entrap and/or encapsulate a local anesthetic agent (e.g., any of the exemplary local anesthetic agents provided herein) within its solid lipid core matrix. A lipid nanoparticle can be used to formulate a local anesthetic agent (e.g., any of the exemplary local anesthetic agents provided herein). A lipid nanoparticle can have an average diameter of, e.g., between about 10 nm to about 1,000 nm (e.g., between about 10 nm to about 900 nm, between about 10 nm to about 800 nm, between to about 10 nm to about 700 nm, between about 10 nm to about 600 nm, between about 10 nm to about 500 nm, between about 10 nm to about 400 nm, between about 10 nm to about 300 nm, between about 10 nm to about 200 nm, between about 10 nm to about 150 nm, between about 10 nm to about 100 nm, between about 10 nm to about 50 nm, between about 15 nm to about 1,000 nm, between about 15 nm to about 900 nm, between about 15 nm to about 800 nm, between about 15 nm to about 700 nm, between about 15 nm to about 600 nm, between about 15 nm to about 500 nm, between about 15 nm to about 400 nm, between about 15 nm to about 300 nm, between about 15 nm to about 200 nm, between about 15 nm to about 150 nm, between about 15 nm to about 100 nm, between about 15 nm to about 50 nm, between about 20 nm to about 1000 nm, between about 20 nm to about 900 nm, between about 20 nm to about 800 nm, between about 20 nm to about 700 nm, between about 20 nm to about 600 nm, between about 20 nm to about 500 nm, between about 20 nm to about 400 nm, between about 20 nm to about 300 nm, between about 20 nm to about 200 nm, between about 20 nm to about 150 nm, between about 20 nm to about 100 nm, between about 20 nm to about 50 nm, between about 25 nm to about 1,000 nm, between about 25 nm to about 900 nm, between about 25 nm to about 800 nm, between about 25 nm to about 700 nm, between about 25 nm to about 600 nm, between about 25 nm to about 500 nm, between about 25 nm to about 400 nm, between about 25 nm to about 300 nm, between about 25 nm to about 200 nm, between about 25 nm to about 150 nm, between about 25 nm to about 100 nm, between about 25 nm to about 50 nm, between about 50 nm to about 1,000 nm, between about 50 nm to about 900 nm, between about 50 nm to about 800 nm, between about 50 nm to about 700 nm, between about 50 nm to about 600 nm, between about 50 nm to about 500 nm, between about 50 nm to about 400 nm, between about 50 nm to about 300 nm, between about 50 nm to about 250 nm, between about 50 nm to about 200 nm, between about 50 nm to about 150 nm, between about 50 nm to about 100 nm, between about 100 nm to about 1,000 nm, between about 100 nm to about 900 nm, between about 100 nm to about 800 nm, between about 100 nm to about 700 nm, between about 100 nm to about 600 nm, between about 100 nm to about 500 nm, between about 100 nm to about 400 nm, between about 100 nm to about 300 nm, between about 100 nm to about 200 nm, between about 200 nm to about 1,000 nm, between about 200 nm to about 900 nm, between about 200 nm to about 800 nm, between about 200 nm to about 700 nm, between about 200 nm to about 600 nm, between about 200 nm to about 500 nm, between about 200 nm to about 400 nm, between about 200 nm to about 300 nm, between about 400 nm to about 1,000 nm, between about 400 nm to about 800 nm, between about 400 nm to about 600 nm, between about 600 nm to about 1,000 nm, between about 600 nm to about 800 nm, or between about 800 nm to about 1,000 nm). Additional aspects of lipid nanoparticles that can be used for entrapping and/or encapsulating a drug and methods of making lipid nanoparticles are described in, e.g., Mukherjee et al., Indian J. Pharm. Sci. 71:349-358, 2009; Das et al., AAPS Pharm Sci Tech. 12:62-76, 2011; Souto et al., Methods Enzymol. 464:105-129, 2009; Ekambaram et al., Sci. Rev. Chem. Comm. 2:80-102, 2012; Mehnert et al., Adv. Drug Delivery Rev. 64:83-101, 2012; Garud et al., Int. Curr. Pharmaceutical J. 1:384-393, 2012; Souto et al., Fundamentals of Pharmaceutical Nanosceince, pp. 91-116, Springer, 2013; and Jose et al., Int. J. Pharm. 474:6-13, 2014.

Cyclodextrins are art known and are molecules capable of acting as effective drug delivery vehicles. Cyclodextrins that have a lipophilic inner cavity and a hydrophilic outer surface are capable of entrapping and/or encapsulating a lipophilic pharmaceutical agent (e.g., any of the exemplary local anesthetic agents provided herein). Cyclodextrins are able to improve the solubility of lipophilic pharmaceutical agents (e.g., a free-base form of a local anesthetic agent) in a pharmaceutical composition (e.g., an aqueous spray solution). Non-limiting examples of a cyclodextrin that can be included in any of the pharmaceutical compositions herein include: cyclodextrin, beta-cyclodextrin, hydroxypropyl beta-cyclodextrin, and dimethyl-beta-cyclodextrin. Additional examples of cyclodextrins are known in the art. Additional aspects of cyclodextrins that can be used for entrapping and/or encapsulating a drug and methods of making cyclodextrin-drug complexes are known in the art (e.g., Challa et al., AAPS Pharm Sci Tech. 45:329-357, 2005).

In some embodiments of any of the pharmaceutical compositions provided herein, an additional local anesthetic agent (e.g., a second, a third, or a fourth local anesthetic agent) can act as a mucosal absorption enhancer, e.g. through synergistic effects (e.g., a eutectic system).

Non-limiting assays for identifying a mucosal absorption enhancer that is capable of increasing permeability or absorption (e.g., paracellular transport and/or transcellular transport) of a pharmaceutical agent (e.g., a local anesthetic agent) across a cell monoloayer (e.g., a Caco-2 cell monolayer or a EpiAirway™ cell monolayer) or mucosal epithelium (e.g., nasal mucosa) are described in the Examples. Additional assays for identifying a mucosal absorption enhancer that is capable of increasing permeability or absorption (e.g., paracellular transport and/or transcellular transport) of a pharmaceutical agent (e.g., a local anesthetic agent) across a cell monolayer (e.g., a Caco-2 monolayer) or a mucosal epithelium (e.g., nasal mucosa) are known in the art. Non-limiting assays for identifying a mucosal absorption enhancer that increases the adhesive strength and/or residence time of a therapeutic agent (e.g., a local anesthetic agent) on a targeted area of a mucosal epithelium (e.g., the nasal mucosa) are described in the Examples. Additional assays for identifying a mucosal absorption enhancer that increases the adhesive strength and/or residence time of a therapeutic agent (e.g., a local anesthetic agent) on a targeted area of a mucosal epithelium (e.g., the nasal mucosa) are known in the art.

The concentration of mucosal absorption agent(s) in any of the pharmaceutical compositions provided herein or in any of the first pharmaceutical compositions and/or second pharmaceutical compositions provided herein can be determined by taking into account the specific mucosal absorption enhancer(s) that are included in the composition, as well as the desired strength and activity of the pharmaceutical composition or the desired strength and activity of the first pharmaceutical composition and/or second pharmaceutical composition, and the specific local anesthetic agent(s) included in the pharmaceutical composition or included in the first pharmaceutical composition and/or the second pharmaceutical composition. Any of the mucosal absorption enhancer(s) described herein can be present in the pharmaceutical composition (e.g., a liquid, gel, or paste) or present in the first pharmaceutical composition (e.g., a liquid, gel, or paste) and/or present in the second pharmaceutical composition, at a concentration of between about 0.001% to about 65% (e.g., between about 0.001% to about 60%, between about 0.001% to about 55%, between about 0.001% to about 50%, between about 0.001% to about 45%, between about 0.001% to about 40%, between about 0.001% to about 35%, between about 0.001% to about 30%, between about 0.001% to about 25%, between about 0.001% to about 20%, between about 0.001% to about 15%, between about 0.001% to about 10%, between about 0.001% to about 8%, between about 0.001% to about 6%, between about 0.001% to about 5%, between about 0.001% to about 4%, between about 0.001% to about 3%, between about 0.001% to about 2%, between about 0.001% to about 1%, between about 0.1% to about 65%, between about 0.1% to about 60%, between about 0.1% to about 55%, between about 0.1% to about 50%, between about 0.1% to about 45%, between about 0.1% to about 40%, between about 0.1% to about 35%, between about 0.1% to about 30%, between about 0.1% to about 25%, between about 0.1% to about 20%, between about 0.1% to about 15%, between about 0.1% to about 10%, between about 0.1% to about 8%, between about 0.1% to about 6%, between about 0.1% to about 5%, between about 0.1% to about 4%, between about 0.1% to about 3%, between about 0.1% to about 2%, between about 0.1% to about 1%, between about 1% to about 65%, between about 1% to about 60%, between about 1% to about 55%, between about 1% to about 50%, between about 1% to about 45%, between about 1% to about 40%, between about 1% to about 35%, between about 1% to about 30%, between about 1% to about 25%, between about 1% to about 20%, between about 1% to about 15%, between about 1% to about 10%, between about 1% to about 8%, between about 1% to about 6%, between about 1% to about 5%, between about 1% to about 4%, between about 1% to about 3%, between about 1% to about 2.5%, between about 1% to about 2.0%, or between about 1% to about 1.5%) by weight of the pharmaceutical composition, the first pharmaceutical composition, or second pharmaceutical composition (e.g., w/w or w/v).

### Drug Delivery Vehicles

Drug delivery vehicles are carrier systems that can be included in pharmaceutical compositions in order to, e.g., improve drug action and safety, often by improving in vivo biodistribution of the drug. Non-limiting examples of drug delivery vehicles include liposomes, microemulsions, emulsions, micelles, lipid nanoparticles, and cyclodextrins. Some embodiments of any of the pharmaceutical compositions described herein can include a drug delivery vehicle (e.g., any of the drug delivery vehicles described herein or known in the art).

In some embodiments of any of the pharmaceutical compositions described herein, the free-base form of a local anesthetic agent is entrapped and/or encapsulated by the drug delivery vehicle. In some embodiments of any of the pharmaceutical compositions described herein, the free-base form of a local anesthetic agent is formulated in the drug delivery vehicle.

A free-base form of a local anesthetic agent free-base that is encapsulated and/or entrapped by a drug delivery vehicle (or formulated using a drug delivery vehicle) can, e.g., allow for an improvement in the delivery of the local anesthetic agent to the trigeminal nerve pathway without the use of a vasoconstrictor. Without being bound by theory, it is believed that utilizing a drug delivery vehicle (e.g., liposome) to formulate, encapsulate, and/or entrap the free-base form of the local anesthetic agent can allow for an increase in the local anesthetic agent's permeation across the nasal mucosa and can allow for an increase in the retention of the local anesthetic agent in the trigeminal nerve pathway. This can provide for, e.g., both a decrease in the systemic delivery of the local anesthetic agent and an increase in its absorption into the target nerve tissue (e.g., the posterior superior alveolar nerve). This targeted drug delivery can provide for, e.g., an improvement in the therapeutic action and safety profile of intranasally administered local anesthetic agents (e.g., a local anesthetic agent in its free-base form). Drug delivery vehicles that encapsulate and/or entrap a local anesthetic agent in its free-base form can also, e.g., provide for an increase the depth of anesthesia, thereby stopping pain signals in a greater number (e.g., an increase of one, two, three, four, five, six, seven, eight, nine, or ten) of nerves (e.g., as compared to the number of nerves with stopped pain signals after intranasal administration of a reference composition including a local anesthetic agent salt and a vasoconstrictor (where the composition does not include a significant or detectable level of local anesthetic agent in its free-base form)). Optionally, drug delivery vehicles can be designed to be mucoadhesive, thereby providing for an increase in residence time at the site of delivery on the nasal mucosa and further improving drug absorption.

The concentration of drug delivery vehicle in any of the pharmaceutical compositions provided herein can be determined by taking into account the specific drug delivery vehicle that is included in the composition, as well as the desired strength and activity of the pharmaceutical composition and the specific local anesthetic agent(s) included in the pharmaceutical composition. Any of the drug delivery vehicles described herein can be present in the pharmaceutical composition (e.g., a liquid, gel, or paste) at a concentration of between about 0.001% to about 65% (e.g., between about 0.001% to about 60%, between about 0.001% to about 55%, between about 0.001% to about 50%, between about 0.001% to about 45%, between about 0.001% to about 40%, between about 0.001% to about 35%, between about 0.001% to about 30%, between about 0.001% to about 25%, between about 0.001% to about 20%, between about 0.001% to about 15%, between about 0.001% to about 10%, between about 0.001% to about 8%, between about 0.001% to about 6%, between about 0.001% to about 5%, between about 0.001% to about 4%, between about 0.001% to about 3%, between about 0.001% to about 2%, between about 0.001% to about 1%, between about 0.1% to about 65%, between about 0.1% to about 60%, between about 0.1% to about 55%, between about 0.1% to about 50%, between about 0.1% to about 45%, between about 0.1% to about 40%, between about 0.1% to about 35%, between about 0.1% to about 30%, between about 0.1% to about 25%, between about 0.1% to about 20%, between about 0.1% to about 15%, between about 0.1% to about 10%, between about 0.1% to about 8%, between about 0.1% to about 6%, between about 0.1% to about 5%, between about 0.1% to about 4%, between about 0.1% to about 3%, between about 0.1% to about 2%, between about 0.1% to about 1%, between about 1% to about 65%, between about 1% to about 60%, between about 1% to about 55%, between about 1% to about 50%, between about 1% to about 45%, between about 1% to about 40%, between about 1% to about 35%, between about 1% to about 30%, between about 1% to about 25%, between about 1% to about 20%, between about 1% to about 15%, between about 1% to about 10%, between about 1% to about 8%, between about 1% to about 6%, between about 1% to about 5%, between about 1% to about 4%, between about 1% to about 3%, between about 1% to about 2.5%, between about 1% to about 2.0%, or between about 1% to about 1.5%) by weight of the pharmaceutical composition (e.g., w/w or w/v).

A liposome is a vesicular lipid bilayer that encloses an aqueous region. Liposomes can include one or more of the following: a phospholipid, an excipient, and a local anesthetic agent (e.g., a local anesthetic agent in its free-base form). A liposome can encapsulate a local anesthetic agent (e.g., a local anesthetic agent in its free-base form) within the aqueous region of the liposome and/or can entrap the local anesthetic agent (e.g., a local anesthetic agent in its free-base form) within the lipid bilayer of the liposome. For example, liposomes are capable of carrying (e.g., entrapping and/or encapsulating), e.g., lipophilic drugs within their lipid bilayer and hydrophilic drugs within their aqueous inner region. Any of the liposomes can have a net charge in any of the pharmaceutical compositions described herein that is cationic, neutral, or anionic.

The lipid bilayer of a liposome can be composed, at least in part, of one or more phospholipids (e.g., one or more synthetic phospholipids and/or naturally-occurring phospholipids) that can have a net charge (e.g., a net positive or net negative charge) or can be neutral in charge. Non-limiting examples of a phospholipid that can be included in any of the pharmaceutical compositions herein include: phosphatidylcholines, lysophosphatidylcholines, phosphatidylserines, phosphatidylethanolamines, phosphatidylglycerols, and phosphatidylinositols. As can be appreciated by those skilled in the art, one or more of any of the exemplary phospholipids described herein or known in the art can be present in the lipid bilayer of a liposome.

Liposomes can also, e.g., contain an excipient (e.g., cholesterol) within the lipid bilayer in order to alter their characteristics (e.g., increase stability and/or increase drug encapsulation and/or entrapment efficiency) and/or, e.g., be coated (e.g., surface modification) with an excipient (e.g., a polymer, such as, e.g., a chitosan)) in order to alter their characteristics (e.g., increase mucoadhesion). Non-limiting examples of an excipient that can be included in any of the pharmaceutical compositions herein include a cholesterol, a stearylamine, a stearic acid, a tocopherol, a polymer, a chitosan, and a polyacrylic acid. In some examples, a liposome can include a phospholipid and an excipient (e.g., and an entrapped and/or encapsulated local anesthetic agent in its free-base form).

Liposomes can have an average diameter of between about 2.5 nM and about 3,000 nm (e.g., any of the ranges for liposome diameters describes herein). Liposomes are categorized by the number of lamellae (e.g., multiple lamellae) they have and by their size (e.g., an average diameter of 200 nm). Non-limiting examples of a liposome that can be included in any of the pharmaceutical compositions herein include: a small unilamellar vesicle, a large unilamellar vesicle, a giant unilamellar vesicle, a multilamellar vesicle, and a multivesicular vesicle. Exemplary features of small unilamellar vesicles (see, e.g., Decker et al., J. Liposome Res. 23:154-165, 2013; Lin et al., Langmuir 28:689-700, 2012; and Lohse et al., J. Am. Chem. Soc. 130:14372-14373, 2008), large unilamellar vesicles (see, e.g., Allen et al., FEBS Lett 223:42-46, 1987; U.S. Pat. No. 4,078,052; Hope et al., Biochim. Biophys. Acta Biomembranes 812:55-65, 1985; and U.S. Patent Application Publication No. 2008/0241233), giant unilamellar vesicles (see, e.g., Moscho et al., Proc. Natl. Acad. Sci. U.S.A. 93:11443-11447, 1996; Pott et al., Chem. Phys. Lipids 154:115-119, 2008; and Carvalho et al., Biophys. 1 95:4348-4360, 2008), multilamellar vesicles (see, e.g., Moon et al., Nat. Mater. 10:243-251, 2011; Hope et al., Chem. Phys. Lipids 40:89-107, 1986; Vyas et al., Pharm. Acta Hely. 74:51-58, 1999; and Kim et al., Biochim. Biophys. Acta Biomembranes 812:793-801, 1985), and multivesicular vesicles (see, e.g., Cohen et al., J. Materials Chem. B 1:4619-4627, 2013; U.S. Pat. No. 5,993,850; Kim et al., Biochim. Biophys. Acta Biomembranes 728:339-348, 1983; and Qiu et al., Acta Pharmacologica Sinica 26:1395-1401, 2005) are known in the art. For example, small unilamellar vesicles have one lamellae and typically have an average diameter of about 20 nm to about 100 nm (e.g., any of small unilamellar vesicle average diameter ranges described herein). Large unilamellar vesicles have one lamellae and typically have an average diameter of between about 100 nm to about 400 nm (e.g., any of the large unilamellar vesicle average diameter ranges described herein). Multilamellar vesicles have multiple lamellae and typically have an average diameter of between about 200 nm to about 3,000 nm (e.g., any of the multilamellar vesicle average diameter ranges described herein). Additional aspects of liposomes that can be used for entrapping and encapsulating a drug and methods of making liposomes are described in, e.g., Akbarzadeh et al., Nanoscale Res. Lett. 8:102, 2013; Anarnath et al., Int. I Pharm. 154:123-140, 1997; Bozzuto et al., Int. J. Nanomedicine 10:975-999, 2015; van der Meel et al., J. Control Release 195:72-85, 2014; Mittal et al., J. Drug Target. 22:372-386, 2014; Kroon et al., Cancer Treat Rev. 40:578-584, 2014; Karn et al., Nanomedicine 8:1529-1548, 2013; Arias, Expert Opin. Ther. Pat. 23:1399-1414, 2013; and Eloy et al., Colloids Surf. B Biointerfaces 123C:345-363, 2014.

Microemulsions are art known and are generally systems including water, oil, and surfactant that can, e.g., be utilized to carry drugs either in the oil phase or aqueous phase. Microemulsions can, e.g., optionally include a co-surfactant. The surfactant, and optionally the co-surfactant, in the microemulsion stabilize the dispersed phase. Microemulsions are thermodynamically stable, colloidal mixtures that can also be, e.g., clear in solution. A microemulsion can be classified by its continuous phase and dispersed phase. For example, a microemulsion can be oil-in-water (o/w), water-in-oil (w/o), or bi-continuous. A microemulsion can include droplets having an average diameter of between, e.g., about 1 nm to about 100 nm (e.g., any of the droplet average diameter ranges for microemulsions described herein). A local anesthetic agent (e.g., a local anesthetic agent in its free-base form) can be entrapped by one or more droplets present in a microemulsion. Additional aspects of microemulsions that can be used for entrapping and/or encapsulating a drug and methods of making microemlusions are described in, e.g., Panapisal et al., AAPS Pharma Sci Tech 13:389-399, 2012; Baboota et al., PDA J. Pharm. Sci. Technol. 61:276-285, 2007; Mandal et al., Int. J. Pharm. Sci. Nanotechnol. 3:1214-1219, 2011; Sharma et al., J. Pharm Drug. Deliv. Res. 1:3, 2012; Todosijevic et al., Colloid Polymer Sci. 292:3061-3076, 2014; Patel et al., Int. J. Biomed. Pharm. Sci. 7:20-27, 2013; Abdelrahim et al., Int. J. Nov. Drug Deliv. Tech. 1:208-212, 2011; and Yu et al., J. Pharm. Sci. 100: 933-941, 2011. Additional non-limiting examples of the properties and composition of microemulsions are described herein.

Emulsions are art known and are a dispersion of droplets of one liquid in another liquid in which it is normally immiscible. An emulsion is a colloid. An emulsion can, e.g., entrap a local anesthetic agent (e.g., a local anesthetic agent in its free-base form) in one or more of its droplets. The droplets in an emulsion can have an average diameter of between about 10 nm to about 100 μm (e.g., any of the droplet average diameter ranges described herein for emulsions). Additional aspects of emulsions that can be used for entrapping and/or encapsulating a drug and methods of making emulsions are described in, e.g., Collins-Gold et al., Adv. Drug Delivery Rev. 5:189-208, 1990; Washington, Adv. Drug Delivery Rev. 20:131-145, 1996; Davis et al., Ann. NY Acad Sci. 507:75-88, 1987; and Wadhwa et al., Acta Poloniae Pharm. 69:179-191, 2012.

Micelles are art known and include a lipid monolayer enclosing a hydrophobic region. A micelle can, e.g., encapsulate a local anesthetic agent (e.g., a local anesthetic agent in its free-base form) in its enclosed hydrophobic region and/or can entrap a local anesthetic agent (e.g., a local anesthetic agent in its free-base form) in its lipid monolayer. Micelles can include amphiphilic molecules, e.g., amphiphilic di- or tri-block copolymers. Non-limiting aspects of micelles are described herein. Micelles can have an average diameter of, e.g., between about 2.0 nm to about 30 nm (e.g., any of the micelle average diameter ranges described herein). Additional aspects of micelles that can be used for entrapping and/or encapsulating a drug and methods of making micelles are described in, e.g., Wang et al., Int. J. Nanomed. 7:4487-4497, 2012; Bachhav et al., J. Controlled Release 153:126-132, 2011; WO 03/047493; U.S. Patent Application Publication No. 2009/0036389; U.S. Patent Application Publication No. 2008/0248097; and Liu et al., Curr. Pharmaceutical Design 12(36):4685-4701, 2006.

Lipid nanoparticles (also known as solid lipid nanoparticles) are art known and are particles that include a solid lipid core matrix that is stabilized by a surfactant. A lipid nanoparticle can entrap and/or encapsulate a local anesthetic agent (e.g., a local anesthetic agent in its free-base form) within its solid lipid core matrix. A lipid nanoparticle can be used to formulate a local anesthetic agent (e.g., a local anesthetic agent in its free-base form). A lipid nanoparticle can have an average diameter of, e.g., between about 10 nm to about 1,000 nm (e.g., any of the lipid nanoparticle average diameter ranges described herein). Additional aspects of lipid nanoparticles that can be used for entrapping and/or encapsulating a drug and methods of making lipid nanoparticles are described in, e.g., Mukherjee et al., Indian J. Pharm. Sci. 71:349-358, 2009; Das et al., AAPS Pharm Sci Tech. 12:62-76, 2011; Souto et al., Methods Enzymol. 464:105-129, 2009; Ekambaram et al., Sci. Rev. Chem. Comm. 2:80-102, 2012; Mehnert et al., Adv. Drug Delivery Rev. 64:83-101, 2012; Garud et al., Int. Curr. Pharmaceutical J. 1:384-393, 2012; Souto et al., Fundamentals of Pharmaceutical Nanosceince, pp. 91-116, Springer, 2013; and Jose et al., Int. J. Pharm. 474:6-13, 2014.

Cyclodextrins are art known and are molecules capable of acting as effective drug delivery vehicles. Cyclodextrins that have a lipophilic inner cavity and a hydrophilic outer surface are capable of entrapping and/or encapsulating a lipophilic pharmaceutical agent (e.g., a free-base form of a local anesthetic agent). Cyclodextrins are able to improve the solubility of lipophilic pharmaceutical agents (e.g., a free-base form of a local anesthetic agent) in a pharmaceutical composition (e.g., an aqueous spray solution). Non-limiting examples of a cyclodextrin that can be included in any of the pharmaceutical compositions herein include: cyclodextrin, beta-cyclodextrin, hydroxypropyl beta-cyclodextrin, and dimethyl-beta-cyclodextrin. Additional examples of cyclodextrins are known in the art. Additional aspects of cyclodextrins that can be used for entrapping and/or encapsulating a drug and methods of making cyclodextrin-drug complexes are known in the art (e.g., Challa et al., AAPS Pharm Sci Tech. 45:329-357, 2005).

Non-limiting assays for identifying a drug delivery vehicle that is capable of providing equivalent or increased absorption of a pharmaceutical agent (e.g., a local anesthetic agent in its free-base form) across a cell monolayer (e.g., a Caco-2 cell monolayer) or mucosal epithelium (e.g., nasal mucosa) are described in the Examples. Additional assays for identifying a drug delivery vehicle that is capable of providing equivalent or increased absorption of a pharmaceutical agent (e.g., a local anesthetic agent in its free-base form) across a cell monolayer (e.g., a Caco-2 cell monolayer) or mucosal epithelium (e.g., nasal mucosa) are known in the art. Non-limiting assays for identifying a drug delivery vehicle that increases the adhesive strength and/or residence time of a therapeutic agent (e.g., a local anesthetic agent in its free-base form) on a targeted area of a mucosal epithelium (e.g., the nasal mucosa) are described in the Examples. Additional assays for identifying a drug delivery vehicle that increases the adhesive strength and/or residence time of a therapeutic agent (e.g., a local anesthetic agent in its free-base form) on a targeted area of a mucosal epithelium (e.g., the nasal mucosa) are known in the art. Non-limiting assays for identifying a drug delivery vehicle that is capable of providing equivalent or increased absorption of a pharmaceutical agent (e.g., a local anesthetic agent in its free-base form) into the trigeminal nerve pathway (e.g., the anterior superior alveolar nerve) are described in the Examples.

Additional assays for identifying a drug delivery vehicle that is capable of providing equivalent or increased absorption of a pharmaceutical agent (e.g., a local anesthetic agent in its free-base form) into the trigeminal nerve pathway (e.g., the anterior superior alveolar nerve) are known in the art.

### Vasoconstrictors

Vasoconstrictor(s), when included in any of the pharmaceutical compositions, first pharmaceutical compositions, and/or second pharmaceutical compositions provided herein, can inhibit or reduce the systemic absorption of the local anesthetic agent following administration of the pharmaceutical composition. Without being bound by any theory, it is believed that the vasoconstrictor(s) present in any of the pharmaceutical compositions, first pharmaceutical compositions, and/or second pharmaceutical compositions provided herein, will constrict the extensive vascular system within the nasal mucosa more so than the blood supply within the trigeminal nerve pathway (as the trigeminal nerve pathway's blood supply is contained within the nerve tissue itself, it is thought to be less exposed to the delivery of the vasoconstrictor).

Non-limiting examples of vasoconstrictor(s) that can be included in any of the pharmaceutical compositions, first pharmaceutical compositions, and/or second pharmaceutical compositions provided herein include: oxymetazoline, phenylephrine, naphazoline, propylhexadrine, levodesoxyephedrine, epinephrine, norepinephrine, and a salt thereof. Additional examples of vasoconstrictor(s) that can be included in any of pharmaceutical compositions, first pharmaceutical compositions, and/or second pharmaceutical compositions described herein are well known in the art. Any of the vasoconstrictor(s) described herein can be present in the pharmaceutical composition (e.g., a liquid, gel, or paste), the first pharmaceutical composition (e.g., a liquid, gel, or paste), and/or second pharmaceutical composition (e.g., a liquid, gel, or paste) at a concentration of between about 0.001% to about 20% (e.g., between about 0.001% to about 15%, between about 0.001% to about 10%, between about 0.001% to about 8%, between about 0.001% to about 6%, between about 0.001% to about 5%, between about 0.001% to about 4%, between about 0.001% to about 3%, between about 0.001% to about 2%, between about 0.001% to about 1%, between about 0.001% to about 0.5%, between about 0.1% to about 20%, between about 0.1% to about 15%, between about 0.1% to about 10%, between about 0.1% to about 8%, between about 0.1% to about 6%, between about 0.1% to about 5%, between about 0.1% to about 4.5%, between about 0.1% to about 4.0%, between about 0.1% to about 3.5%, between about 0.1% to about 3.0%, between about 0.1% to about 2.5%, between about 0.1% to about 2.0%, between about 0.1% to about 1.5%, between about 0.1% to about 1.0%, between about 0.1% to about 0.5%, between about 0.5% to about 20%, between about 0.5% to about 15%, between about 0.5% to about 10%, between about 0.5% to about 8%, between about 0.5% to about 6%, between about 0.5% to about 5%, between about 0.5% to about 4.5%, between about 0.5% to about 4.0%, between about 0.5% to about 3.5%, between about 0.5% to about 3.0%, between about 0.5% to about 2.5%, between about 0.5% to about 2.0%, between about 0.5% to about 1.5%, or between about 0.5% to about 1.0%) by weight of the composition (e.g., w/w or w/v).

### Preservative

Non-limiting examples of preservative(s) that can be included in any of the pharmaceutical compositions, first pharmaceutical compositions, and/or second pharmaceutical compositions provided herein include: sorbitol, mannitol, ethanol, benzyl alcohol, isopropanol, cresol, chlorocresol, phenol, and benzalkonium chloride. Additional examples of preservative(s) that can be included in any of pharmaceutical compositions, first pharmaceutical compositions, and/or second pharmaceutical compositions described herein are well known in the art. The concentration of the preservative(s) present in any of the pharmaceutical compositions, first pharmaceutical compositions, and/or second pharmaceutical compositions provided herein will depend on the specific preservative. Any of the preservative(s) described herein can be present in the pharmaceutical composition (e.g., a liquid, gel, or paste), first pharmaceutical composition (e.g., a liquid, gel, or paste), and/or a second pharmaceutical composition (e.g., a liquid, gel, or paste) at a concentration of between about 0.01% to about 10% (e.g., between about 0.01% to about 8%, between about 0.01% to about 6%, between about 0.01% to about 5%, between about 0.01% to about 4%, between about 0.01% to about 4.5%, between about 0.01% to about 4.0%, between about 0.01% to about 3.5%, between about 0.01% to about 3.0%, between about 0.01% to about 2.5%, between about 0.01% to about 2.0%, between about 0.01% to about 1.5%, between about 0.01% to about 1.0%, between about 0.01% to about 0.5%, between about 0.1% to about 10%, between about 0.1% and about 8%, between about 0.1% to about 6%, between about 0.1% to about 5%, between about 0.1% to about 4.5%, between about 0.1% to about 4.0%, between about 0.1% to about 3.5%, between about 0.1% to about 3.0%, between about 0.1% to about 2.5%, between about 0.1% to about 2.0%, between about 0.1% to about 1.5%, between about 0.1% to about 1.0%, or between about 0.1% to about 0.5%) by weight of the composition (e.g., w/w or w/v).

### Viscosity Enhancing Agent

A viscosity enhancing agent, when included in any of the pharmaceutical compositions, first pharmaceutical compositions, and/or second pharmaceutical compositions provided herein, can control (e.g., reduce) the mobility of the pharmaceutical composition, the first pharmaceutical composition, and/or second pharmaceutical composition after placement of the pharmaceutical composition, the first pharmaceutical composition, and/or second pharmaceutical composition onto an area of the nasal tissue (e.g., the nasal mucosa). For example, a viscosity enhancing agent, when included in any of the pharmaceutical compositions, first pharmaceutical compositions, and/or second pharmaceutical compositions provided herein, can control (e.g., reduce) the amount of the pharmaceutical composition, the first pharmaceutical composition, and/or the second pharmaceutical composition that either drips out of the nostril(s) or into the throat of a subject (after intranasal administration of the pharmaceutical composition, the first pharmaceutical composition, and/or the second pharmaceutical composition). A viscosity enhancing agent can be a mucoadhesive agent. The inclusion of a viscosity enhancing agent in any of the pharmaceutical compositions, first pharmaceutical compositions, and/or second pharmaceutical compositions provided herein can, e.g., increase the amount of time the pharmaceutical composition, the first pharmaceutical composition, and/or second pharmaceutical composition (e.g., any of the pharmaceutical compositions, the first pharmaceutical compositions, and/or the second pharmaceutical compositions provided herein) is in contact with the target tissue (e.g., the targeted area of the nasal mucosa).

Non-limiting examples of viscosity enhancing agent(s) that can be included in any of the pharmaceutical compositions, the first pharmaceutical compositions, and/or second pharmaceutical compositions provided herein include: hydroxyethylcellulose, hydroxypropylmethyl cellulose, sodium carboxy methyl cellulose, carbomer homopolymer type a, carbomer homopolymer type b, carbomer, polycarbophil, sodium alginate, xantham gum, smart hydrogel, polyethylene glycol, hydroxycellulose, poloxamer 188, poloxamer 407, starch, aminated gelatin, chitosan, hydroxypropyl methylcellulose, hydroxypropyl cellulose, methylcellulose, carboxymethyl cellulose, ethylcellulose, and microcrystalline celluose. Additional examples of viscosity enhancing agent(s) that can be included in any of pharmaceutical compositions, the first pharmaceutical compositions, and/or second pharmaceutical compositions described herein are well known in the art. Any of the viscosity enhancing agent(s) described herein can be present in the pharmaceutical composition (e.g., a liquid, gel, or paste), the first pharmaceutical composition (e.g., a liquid, gel, or paste), and/or the second pharmaceutical composition (e.g., a liquid, gel, or paste) at a concentration of between about 0.01% to about 10% (e.g., between about 0.01% to about 8%, between about 0.01% to about 6%, between about 0.01% to about 5%, between about 0.01% to about 4%, between about 0.01% to about 4.5%, between about 0.01% to about 4.0%, between about 0.01% to about 3.5%, between about 0.01% to about 3.0%, between about 0.01% to about 2.5%, between about 0.01% to about 2.0%, between about 0.01% to about 1.5%, between about 0.01% to about 1.0%, between about 0.01% to about 0.5%, between about 0.1% to about 10%, between about 0.1% and about 8%, between about 0.1% to about 6%, between about 0.1% to about 5%, between about 0.1% to about 4.5%, between about 0.1% to about 4.0%, between about 0.1% to about 3.5%, between about 0.1% to about 3.0%, between about 0.1% to about 2.5%, between about 0.1% to about 2.0%, between about 0.1% to about 1.5%, between about 0.1% to about 1.0%, or between about 0.1% to about 0.5%) by weight of the composition (e.g., w/w or w/v).

### Antihistamines

Non-limiting examples of antihistamine(s) that can be included in any of the pharmaceutical compositions, first pharmaceutical compositions, and/or second pharmaceutical compositions provided herein include: atropine, azelastine, hydroxyzine, desloratadine, cyproheptadine, emadastine, levocabastine, azelastine, carbinoxamine, levocetirizine, fexofenadine, diphenhydramine, bromopheniramine, loratadine, clemastine, chlorpheniramine, certirizine, and a salt thereof. Additional examples of antihistamine(s) that can be included in any of the pharmaceutical compositions, the first pharmaceutical compositions, and/or the second pharmaceutical compositions described herein are well known in the art. Any of the antihistamine(s) described herein can be present in the pharmaceutical composition (e.g., a liquid, gel, or paste), the first pharmaceutical composition (e.g., a liquid, gel, or paste), and/or the second pharmaceutical composition (e.g., a liquid, gel, or paste) at a concentration of between about 0.01% to about 10% (e.g., between about 0.01% to about 8%, between about 0.01% to about 6%, between about 0.01% to about 5%, between about 0.01% to about 4%, between about 0.01% to about 4.5%, between about 0.01% to about 4.0%, between about 0.01% to about 3.5%, between about 0.01% to about 3.0%, between about 0.01% to about 2.5%, between about 0.01% to about 2.0%, between about 0.01% to about 1.5%, between about 0.01% to about 1.0%, between about 0.01% to about 0.5%, between about 0.1% to about 10%, between about 0.1% and about 8%, between about 0.1% to about 6%, between about 0.1% to about 5%, between about 0.1% to about 4.5%, between about 0.1% to about 4.0%, between about 0.1% to about 3.5%, between about 0.1% to about 3.0%, between about 0.1% to about 2.5%, between about 0.1% to about 2.0%, between about 0.1% to about 1.5%, between about 0.1% to about 1.0%, or between about 0.1% to about 0.5%) by weight of the composition (e.g., w/w or w/v).

### Pharmaceutically Acceptable Carriers

Any of the pharmaceutical compositions, first pharmaceutical compositions, and/or second pharmaceutical compositions provided herein can include a pharmaceutically acceptable carrier (e.g., a non-naturally occurring pharmaceutically acceptable carrier). A pharmaceutically acceptable carrier can be included in any of the pharmaceutical compositions, first pharmaceutical compositions, and/or second pharmaceutical compositions provided herein in order to, e.g., control the dosage, absorption rate, or any other desired property (e.g., pH) of the pharmaceutical composition, the first pharmaceutical composition, and/or the second pharmaceutical composition. Non-limiting examples of pharmaceutically acceptable carriers that can be included in the pharmaceutical compositions, the first pharmaceutical compositions, and/or second pharmaceutical compositions provided herein include: aqueous carriers (e.g., water, buffered solutions, e.g., phosphate buffered solutions or citrate buffered solutions, sugar alcohols, alcohols, e.g., ethanol, or any other solvent that is biologically compatible with the nasal mucosa), gel carriers, emulsifiers, surfactants, time release-vehicles (e.g., micelles, liposomes, microemulsions, solid lipid nanoparticles, microparticles, or nanoparticles), colloidal systems, microparticles, and nanoparticles. In some embodiments of any of the compositions described herein, the mucosal absorption enhancer, such as a micelle, a liposome, a microemulsion, a solid lipid nanoparticle, or a transferosome, encapsules or coats or partially coats a local anesthetic agent.

### Methods of Anesthetizing Nerve Tissue in the Trigeminal Nerve Pathway

Provided herein are methods of anesthetizing nerve tissue in the trigeminal nerve pathway (e.g., one or more of any of the nerve tissues present in the trigeminal nerve pathway described herein or known in the art) in a subject (e.g., a human) in need thereof (e.g., any of the subjects described herein) that include: intranasally administering a pharmaceutical composition (e.g., any of the pharmaceutical compositions provided herein) to a subject in need thereof. Also provided are methods of anesthetizing nerve tissue in the trigeminal nerve pathway (e.g., one or more of any of the nerve tissues present in the trigeminal nerve pathway described herein or known in the art) in a subject (e.g., a human) in need thereof (e.g., any of the subjects described herein) that include intranasally administering (1) a first pharmaceutical composition comprising a local anesthetic agent (e.g., any of the first pharmaceutical compositions described herein), and (2) a second pharmaceutical composition comprising a mucosal absorption enhancer to a subject in need thereof (e.g., any of the second pharmaceutical compositions described herein), wherein the local anesthetic agent and the mucosal absorption enhancer are administered in amounts sufficient to anesthetize nerve tissue in the trigeminal nerve pathway upon administration. In some embodiments, the second pharmaceutical composition containing the mucosal absorption enhancer is administered to the subject first and the first pharmaceutical composition containing the local anesthetic agent is administered second (e.g., within about 1 hour, within about 50 minutes, within about 45 minutes, within about 40 minutes, within about 35 minutes, within about 30 minutes, within about 25 minutes, within about 20 minutes, within about 15 minutes, within about 10 minutes, within about 5 minutes, within about 2 minutes, or within about 1 minute after the time the second pharmaceutical composition is administered). In some embodiments, the first pharmaceutical composition containing the local anesthetic agent is administered first and the second pharmaceutical composition containing a mucosal absorption agent is administered second (e.g., within about 1 hour, within about 50 minutes, within about 45 minutes, within about 40 minutes, within about 35 minutes, within about 30 minutes, within about 25 minutes, within about 20 minutes, within about 15 minutes, within about 10 minutes, within about 5 minutes, within about 2 minutes, or within about 1 minute after the time the first pharmaceutical composition is administered). In some embodiments, the first pharmaceutical composition and the second pharmaceutical composition are administered at substantially the same time.

Also provided are methods of anesthetizing nerve tissue in the trigeminal nerve pathway (e.g., one or more of any of the nerve tissues present in the trigeminal nerve pathway described herein or known in the art) in a subject (e.g., a human) in need thereof (e.g., any of the subject described herein) that include intranasally administering (1) a first pharmaceutical composition comprising a mucosal absorption enhancer (e.g., any of the first pharmaceutical compositions described herein), and (2) a second pharmaceutical composition comprising a local anesthetic agent to a subject in need thereof (e.g., any of the second pharmaceutical compositions described herein), wherein the local anesthetic agent and the mucosal absorption agent are administered in amounts sufficient to anesthetize nerve tissue in the trigeminal nerve tissue in the trigeminal nerve pathway upon administration. In some other embodiments, the first pharmaceutical composition containing the mucosal absorption enhancer is administered to the subject second and the second pharmaceutical composition containing the local anesthetic agent is administered first (e.g., where the first pharmaceutical composition is administered within about 1 hour, within about 50 minutes, within about 45 minutes, within about 40 minutes, within about 35 minutes, within about 30 minutes, within about 25 minutes, within about 20 minutes, within about 15 minutes, within about 10 minutes, within about 5 minutes, within about 2 minutes, or within about 1 minute after the time the second pharmaceutical composition is administered). In some embodiments, the first pharmaceutical composition containing a mucosal absorption enhancer is administered first and the second pharmaceutical composition containing the local anesthetic agent is administered second (e.g., within about 1 hour, within about 50 minutes, within about 45 minutes, within about 40 minutes, within about 35 minutes, within about 30 minutes, within about 25 minutes, within about 20 minutes, within about 15 minutes, within about 10 minutes, within about 5 minutes, within about 2 minutes, or within about 1 minute after the time the first pharmaceutical composition is administered). In some embodiments, the first pharmaceutical composition and the second pharmaceutical composition are administered at substantially the same time.

The subject in any of these methods can be any of the exemplary subjects described herein. Any of the pharmaceutical compositions, first pharmaceutical compositions, and/or second pharmaceutical compositions described herein can be used in any of the methods described herein. In some embodiments of any of the methods described herein, the pharmaceutical composition or the first and second pharmaceutical compositions are directly administered onto one or more of the following nasal tissues: the inferior nasal turbinate, the middle nasal turbinate, the nasal meatuses, the extreme rear of the nasal cavity, and the maxillary sinus. In some examples of any of the methods described herein, the pharmaceutical composition or the first and second pharmaceutical compositions are directly placed on one or more of the following nasal tissues: inferior nasal turbinate, the middle nasal turbinate, the nasal meatuses, the extreme rear of the nasal cavity, and the maxillary sinus.

The pharmaceutical composition or the first and second pharmaceutical compositions can be formulated and administered, e.g., as a gel, a solution, a spray, a paste, an emulsion, a powder, a colloid, a microparticle or a nanoparticle, to the subject (e.g., a human). In some examples of any of the methods provided herein, the pharmaceutical composition or the first and second pharmaceutical compositions are administered to a subject within 1 hour (e.g., within 50 minutes, within 45 minutes, within 40 minutes, within 35 minutes, within 30 minutes, within 25 minutes, within 20 minutes, within 15 minutes, within 10 minutes, or within 5 minutes) of the start of a surgery (e.g., any of the exemplary types of surgery described herein) or a procedure (e.g., any of the exemplary procedures described herein). Any of the methods described herein can include the administration of one or more (e.g., two, three, four, five, six, seven, eight, or nine) doses of the pharmaceutical composition (e.g., any of the pharmaceutical compositions described herein) or one or more doses of the first and/or second pharmaceutical composition(s) to the subject. When two or more doses are administered to a subject, the individual doses are administered at an interval of between about 1 minute to about 10 minutes (e.g., about 1 minute to about 8 minutes, about 1 minute to about 6 minutes, at about 1 minute to about 4 minutes, or at about 1 minute to about 2 minutes). In some embodiments, the pharmaceutical composition or the first and second pharmaceutical compositions is/are administered by a medical professional or a dental professional (e.g., a hygienist, a dentist, or an oral surgeon). In some examples of any of the methods provided herein, the subject self-administers the pharmaceutical composition or the first and second pharmaceutical compositions (e.g., a subject having mouth pain or nasal pain can, e.g., self-administer any of the pharmaceutical compositions described herein or any of the first and second pharmaceutical compositions described herein).

In any of the methods described herein, at least one dose of the pharmaceutical composition or the first and/or second pharmaceutical compositions can be intranasally administered to a subject in the middle of the performance of a surgery (e.g., any of the exemplary surgeries described herein) or a procedure (e.g., any of the exemplary procedures described herein).

When a first and a second pharmaceutical composition are administered to a subject, the first and second pharmaceutical compositions can have different formulations (e.g., the first pharmaceutical composition is formulated as a spray or a liquid and the second pharmaceutical composition is formulated as a gel, cream, or paste). The first pharmaceutical composition and/or second pharmaceutical composition(s) can contain any of the local anesthetic agents described herein or known in the art. The first pharmaceutical composition and/or second pharmaceutical(s) composition can contain the local anesthetic agents at any of the concentrations described herein. The first pharmaceutical composition and/or second pharmaceutical composition(s) can contain any of the mucosal absorption agents described herein or known in the art. The first pharmaceutical composition and/or second pharmaceutical composition(s) can contain the mucosal absorption agents at any of the concentrations described herein. The first and/or second pharmaceutical composition(s) can further contain any one or more of the following: vasoconstrictor(s) (e.g., any of the exemplary vasoconstrictors described herein or known in the art at any of the exemplary concentrations described herein), preservative(s) (e.g., any of the exemplary preservatives described herein or known in the art at any of the exemplary concentrations described herein), viscosity enhancing agent(s) (e.g., any of the exemplary viscosity enhancing agents described herein or known in the art at any of the exemplary concentrations described herein), pharmaceutically acceptable carrier(s) (e.g., any of the exemplary pharmaceutically acceptable carriers described herein or known in the art at any of the exemplary concentrations described herein), and antihistamine(s) (e.g., any of the antihistamines described herein or known in the art at any of the exemplary concentrations described herein).

Some embodiments of any of the methods described herein provide for the anesthetization of the maxillary dental arch (including the maxillary molars and the pain fibers around the maxillary dental arch). In other examples, the methods described herein provide for the anesthetizing the mandibular dental arch and pain fibers around the mandibular dental arch. Some embodiments of any of the methods described herein provide for the anesthetization of the trigeminal ganglion and one or more tissues innervated by the trigeminal nerve pathway.

In some examples of any of the methods described herein, the subject is in need of oral surgery or a dental procedure, and optionally, such methods further include performing oral surgery or the dental procedure, respectively, on the subject. In some examples of any of the methods described herein, the subject is in need of ocular or eyelid surgery or an ocular or eyelid procedure, and optionally, such methods further include performing ocular or eyelid surgery or the ocular or eyelid procedure, respectively, on the subject. In some embodiments of any of the methods described herein the subject is in need of exterior nose surgery, sinus surgery, or a sinus procedure, and optionally, the method further includes performing exterior nose surgery, sinus surgery, or the sinus procedure, respectively, on the subject. Methods for performing oral surgery, ocular surgery, eyelid surgery, exterior nose surgery, and sinus surgery are well known in the art. Methods for performing a dental procedure, an ocular procedure, an eyelid procedure, and a sinus procedure are well known in the art.

### Methods of Performing a Surgical Procedure

Also provided herein are methods of performing a surgical procedure on a subject in need thereof that include intranasally administering a pharmaceutical composition (e.g., any of the pharmaceutical compositions described herein) to a subject (e.g., a human) in need thereof (e.g., any of the subjects described herein), and making at least one incision, puncture, or suture (or debriding) in a dental pulp, soft tissue in the oral cavity, eye, eye socket, soft tissue of the face, and sinuses.

Also provided herein are methods of performing a surgical procedure on a subject (e.g., a human) in need thereof (e.g., any of the exemplary subjects described herein) that include intranasally administering (1) a first pharmaceutical composition including a local anesthetic agent (e.g., any of the first pharmaceutical compositions described herein) and (2) a second pharmaceutical composition including a mucosal absorption agent (e.g., any of the second pharmaceutical compositions described herein), where the local anesthetic agent and the mucosal absorption agent are present in amounts sufficient to anesthetize nerve tissue in the trigeminal nerve pathway upon administration, and making at least one incision, puncture, or suture (or debriding) in a dental pulp, soft tissue in the oral cavity, eye, eye socket, soft tissue of the face, and sinuses. In some embodiments, the second pharmaceutical composition containing the mucosal absorption enhancer is administered first and the first pharmaceutical composition containing the local anesthetic agent is administered second (e.g., within about 1 hour, within about 50 minutes, within about 45 minutes, within about 40 minutes, within about 35 minutes, within about 30 minutes, within about 25 minutes, within about 20 minutes, within about 15 minutes, within about 10 minutes, within about 5 minutes, within about 2 minutes, or within about 1 minute after the time the second pharmaceutical composition is administered). In some embodiments, the first pharmaceutical composition containing the local anesthetic agent is administered first and the second pharmaceutical composition containing a mucosal absorption agent is administered second (e.g., within about 1 hour, within about 50 minutes, within about 45 minutes, within about 40 minutes, within about 35 minutes, within about 30 minutes, within about 25 minutes, within about 20 minutes, within about 15 minutes, within about 10 minutes, within about 5 minutes, within about 2 minutes, or within about 1 minute after the time the first pharmaceutical composition is administered). In some embodiments, the first pharmaceutical composition and the second pharmaceutical composition are administered at substantially the same time.

Also provided herein are methods of performing a surgical procedure on a subject (e.g., a human) in need thereof (e.g., any of the exemplary subjects described herein) that include intranasally administering (1) a first pharmaceutical composition including a mucosal absorption enhancer (e.g., any of the first pharmaceutical compositions described herein) and (2) a second pharmaceutical composition including a local anesthetic agent (e.g., any of the second pharmaceutical compositions described herein), where the local anesthetic agent and the mucosal absorption enhancer are present in amounts sufficient to anesthetize nerve tissue in the trigeminal nerve pathway upon administration, and making at least one incision, puncture, or suture (or debriding) in a dental pulp, soft tissue in the oral cavity, eye, eye socket, soft tissue of the face, and sinuses. In some embodiments, the second pharmaceutical composition containing the local anesthetic agent is administered first and the first pharmaceutical composition containing the mucosal absorption enhancer is administered second (e.g., within about 1 hour, within about 50 minutes, within about 45 minutes, within about 40 minutes, within about 35 minutes, within about 30 minutes, within about 25 minutes, within about 20 minutes, within about 15 minutes, within about 10 minutes, within about 5 minutes, within about 2 minutes, or within about 1 minute after the time the second pharmaceutical composition is administered). In some embodiments, the first pharmaceutical composition containing the mucosal absorption enhancer is administered first and the second pharmaceutical composition containing the local anesthetic agent is administered second (e.g., within about 1 hour, within about 50 minutes, within about 45 minutes, within about 40 minutes, within about 35 minutes, within about 30 minutes, within about 25 minutes, within about 20 minutes, within about 15 minutes, within about 10 minutes, within about 5 minutes, within about 2 minutes, or within about 1 minute after the time the first pharmaceutical composition is administered). In some embodiment, the first pharmaceutical composition and the second pharmaceutical composition are administered at substantially the same time.

The subject in any of these methods can be any of the exemplary subjects described herein. Any of the pharmaceutical compositions, first pharmaceutical compositions, and second pharmaceutical compositions described herein can be used in any of the methods described herein. In some embodiments of any of the methods described herein, the pharmaceutical composition or the first and second pharmaceutical compositions are directly administered onto one or more of the following nasal tissues: the inferior nasal turbinate, the middle nasal turbinate, the middle nasal turbinate, the nasal meatuses, the extreme rear of the nasal cavity, and the maxillary sinus. In some examples of any of the methods described herein, the pharmaceutical composition or the first and second pharmaceutical compositions are directly placed on one or more of the following nasal tissues: inferior nasal turbinate, the middle nasal turbinate, the nasal meatuses, the extreme rear of the nasal cavity, and the maxillary sinus.

The pharmaceutical composition or the first and second pharmaceutical compositions can be formulated and administered, e.g., as a gel, a solution, a spray, a paste, an emulsion, a powder, a colloid, a microparticle or a nanoparticle, to the subject (e.g., a human). In some examples of any of the methods provided herein, the pharmaceutical composition or the first and second pharmaceutical compositions are administered to a subject within 1 hour (e.g., within 50 minutes, within 45 minutes, within 40 minutes, within 35 minutes, within 30 minutes, within 25 minutes, within 20 minutes, within 15 minutes, within 10 minutes, or within 5 minutes) of the start of a surgery (e.g., any of the exemplary types of surgery described herein) or a procedure (e.g., any of the exemplary procedures described herein). Any of the methods described herein can include the administration of one or more (e.g., two, three, four, five, six, seven, eight, or nine) doses of the pharmaceutical composition (e.g., any of the pharmaceutical compositions described herein) or one or more doses of the first and/or second pharmaceutical composition to the subject. When two or more doses are administered to a subject, the individual doses are administered at an interval of between about 1 minute to about 10 minutes (e.g., about 1 minute to about 8 minutes, about 1 minute to about 6 minutes, at about 1 minute to about 4 minutes, or at about 1 minute to about 2 minutes). In some embodiments, the pharmaceutical composition or the first and second pharmaceutical compositions is administered by a medical professional or a dental professional (e.g., a hygienist, a dentist, or an oral surgeon). In some examples of any of the methods provided herein, the subject self-administers the pharmaceutical composition or the first and second pharmaceutical compositions.

In any of the methods described herein, at least one dose of the pharmaceutical composition or the first and/or second pharmaceutical compositions can be intranasally administered to a subject in the middle of the performance of the surgical procedure (e.g., during any of the exemplary surgeries described herein).

When a first and a second pharmaceutical composition are administered to a subject, the first and second pharmaceutical compositions can have different formulations (e.g., the first pharmaceutical composition is formulated as a spray or a liquid and the second pharmaceutical composition is formulated as a gel, cream, or paste). The first pharmaceutical composition and/or second pharmaceutical composition can contain any of the local anesthetic agents described herein or known in the art. The first pharmaceutical composition and/or second pharmaceutical composition can contain the local anesthetic agents at any of the concentrations described herein. The first pharmaceutical composition and/or second pharmaceutical composition can contain any of the mucosal absorption agents described herein or known in the art. The first pharmaceutical composition and/or second pharmaceutical composition can contain the mucosal absorption agents at any of the concentrations described herein. The first and/or second pharmaceutical compositions can further contain any one or more of the following: vasoconstrictor(s) (e.g., any of the exemplary vasoconstrictors described herein or known in the art at any of the exemplary concentrations described herein), preservative(s) (e.g., any of the exemplary preservatives described herein or known in the art at any of the exemplary concentrations described herein), viscosity enhancing agent(s) (e.g., any of the exemplary viscosity enhancing agents described herein or known in the art at any of the exemplary concentrations described herein), pharmaceutically acceptable carrier(s) (e.g., any of the exemplary pharmaceutically acceptable carriers described herein or known in the art at any of the exemplary concentrations described herein), and antihistamine(s) (e.g., any of the antihistamines described herein or known in the art at any of the exemplary concentrations described herein).

In some examples of any of the methods described herein, the subject is in need of oral surgery, and in such methods the making of at least one incision, puncture, or suture (or the debridement) is performed as part of the oral surgery. The oral surgery can be any of the non-limiting examples of oral surgery described herein (e.g., molar tooth extraction). In some examples of any of the methods described herein, the subject is in need of ocular or eyelid surgery, and in such methods the making of at least one incision, puncture, or suture (or the debridement) is performed as part of the ocular or eyelid surgery. In some embodiments of any of the methods described herein the subject is need of exterior nose surgery or sinus surgery, and in such methods the making of at least one incision, puncture, or suture (or the debridement) is performed as part of the exterior nose surgery or sinus surgery. Methods for making an incision, puncture, or suture (or performing debridement) (e.g., as part of performing oral surgery, ocular surgery, eyelid surgery, exterior nose surgery, or sinus surgery) are well known in the art.

### Kits

Also provided herein are kits that include at least one of any one of the pharmaceutical compositions provided herein. Also provided are kits that include at least one of any of the first pharmaceutical compositions provided herein and/or at least one of any of the second pharmaceutical compositions provided herein. Some examples of any of the kits described herein further include a swap (e.g., a cotton swab) that is impregnated with at least one of the pharmaceutical compositions described herein, at least one of the first pharmaceutical compositions described herein, or at least one of the second pharmaceutical compositions described herein. Some examples of any of the kits described herein further include at least one disposable surgical or procedural tool (e.g., a thread (e.g., nylon thread) for suturing or an abrasive material for debriding tissue). For example, some examples of any of the kits described herein further include at least one device for delivering any of the pharmaceutical compositions described herein, any of the first pharmaceutical compositions described herein, or any of the second pharmaceutical compositions described herein to nasal mucosa (e.g., a swab (e.g., an impregnated swab), a syringe (e.g., a pre-filled syringe), or an impregnated patch).

Some examples of any of the kits described herein further include a lyophilized solid including at least one of the pharmaceutical compositions described herein (e.g., a lyophilized liposome (e.g., multilamellar liposome vesicles), a lipid nanoparticle, or a micelle that entraps and/or encapsulates the local anesthetic agent in its free-base form) and a pharmaceutically acceptable carrier (e.g., water). Some examples include a sterile container (e.g., an ampule or tube) containing the lyophilized solid including at least one of the pharmaceutical compositions described herein (e.g., a lyophilized liposome (e.g., multilamellar liposome vesicles), a lipid nanoparticle, or a micelle that entraps and/or encapsulates the local anesthetic agent in its free-base form) and a sterile pharmaceutically acceptable carrier (e.g., water or phosphate buffered saline) in a sterile container (e.g., a sterile pre-filled syringe or a sterile pre-filled ampule). Some examples include a double chamber pre-filled syringe (e.g., Arte Dual Chamber Prefillable™ Syringe) containing the lyophilized solid in one chamber and a diluent (e.g. a pharmaceutically acceptable carrier) in another chamber. Some embodiments of these kits further include instructions for mixing or solubilizing the lyophilized solid with the pharmaceutically acceptable carrier.

The invention is further described in the following examples, which do not limit the scope of the invention described in the claims.

### EXAMPLES

### Example 1

### Local Anesthetic Absorption Across an Epithelial Cell Monolayer

### Introduction:

A first experiment was performed to test the ability of a mucosal absorption enhancer to promote transport of a local anesthetic agent across a mucosal epithelial cell monolayer. In this experiment, the Caco-2 cell line (an intestinal mucosal cell line) was grown and maintained at a pH of 6.0 for the length of time sufficient to establish tight junctions between the cells in the monolayer. After formation of the monolayer, sodium caprate was administered to the apical side of the Caco-2 cell monolayer at a concentration depending on the given test sample. After 20 minutes, the sodium caprate was washed off of the monolayer. Tetracaine hydrochloride was then administered to the apical side of the Caco-2 cell monolayer at a concentration of 10 mM. An additional test sample including tetracaine but lacking sodium caprate was also administered to the apical side of a separate Caco-2 cell monolayer as a control. The assay duration was 30 minutes. The apical to basolateral permeability rate coefficient of tetracaine across the Caco-2 cell monolayer was determined by LC/MS/MS for each test composition. The permeability rate coefficient (Pₐₚₚ) was based on the percent of the drug that is transported across the epithelial monolayer as a function of time and surface area of the monolayer. Drug permeability across the Caco-2 cell monolayers is a well-known model of transmucosal drug absorption in humans.

### Materials and Methods:

Tetracaine HCl was purchased from Sigma (Lot #SLBF7604; Molecular weight 300.82). Sodium caprate was purchased from Sigma (Lot #SLBH5923V; Molecular weight 194.25).

Caco-2 cells grown in tissue culture flasks were trypsinized, suspended in medium, and the suspensions were applied to wells of a Millipore 96 well Caco-2 plate. The cells were allowed to grow and differentiate for three weeks, feeding at 2-day intervals.

The Caco-2 cells were treated with the excipient (sodium caprate) for 20 minutes at concentrations of 0.2%, 0.5%, 1.0%, and 2.0%. The excipient was then washed off of the Caco-2 cell monolayer. For Apical to Basolateral (A→B) permeability, the test agent (10 mM Tetracaine HCl) was added to the apical (A) side and amount of permeation was determined on the basolateral (B) side. The A-side buffer contained 100 μM Lucifer Yellow (LY) dye and 10 μM Atenolol, in Transport Buffer (1.98 g/L glucose in 10 mM HEPES, 1× Hank's Balanced Salt Solution without Mg2+ and Ca2+) pH 6.0 for test articles. The B-side buffer was standard Transport Buffer, pH 7.4. Caco-2 cells were incubated with these buffers for 0.5 hr, and the receiver side buffer was removed for analysis by LC/MS/MS (propranolol used as internal LC/MS standard).

To verify that the Caco-2 cell monolayers were properly formed (and remained intact during the assay), aliquots of the cell buffers were analyzed by fluorescence and LC/MS/MS to determine the transport of the impermeable dye Lucifer Yellow and atenolol, respectively. Any deviations from control values are reported.

Data are expressed as permeability (Pₐₚₚ): Pₐₚₚ=(dQ/dt)/C₀A

Where dQ/dt is the rate of permeation, C₀ is the initial concentration of test agent, and A is the area of the monolayer.

For analytical method development, the analyte signal was optimized for each compound by ESI positive or negative ionization mode. An MS2 scan or an SIM scan was used to optimize the fragmenter voltage and a product ion analysis was used to identify the best fragment for analysis, and the collision energy was optimized using a product ion or MRM scan. An ionization ranking was assigned indicating the compound's ease of ionization.

Samples were analyzed by LC/MS/MS using an Agilent 6410 mass spectrometer coupled with an Agilent 1200 HPLC and a CTC PAL chilled autosampler, all controlled by MassHunter software (Agilent). After separation on a C18 reverse phase HPLC column (Agilent Zorbax StableBond 3.5 μm, 2.1×30 mm) using an acetonitrile-water gradient system, peaks were analyzed by mass spectrometry (MS) using ESI ionization in MRM mode.

### Results:

TABLE 1
Effects of Mucosal Absorption Enhancer on Permeability of a
Local Anesthetic Agent

| Test Article | Excipient | Mean Pₐₚₚ (10⁻⁶ cm s⁻¹) |
|---|---|---|
| Tetracaine | None | 17.7 |
| | 0.2% Sodium Caprate | 32.2 |
| | 0.5% Sodium Caprate | 55.9 |
| | 1.0% Sodium Caprate | 54.8 |
| | 2.0% Sodium Caprate | 138.7 |

Pₐₚₚ: apparent permeability rate coefficient;
Assay Duration: 0.5 hr

Lucifer Yellow and atenolol transport met quality control criteria for acceptable monolayer integrity at the end of the assay.

### Conclusions:

The data in Table 1 surprisingly show that an exemplary mucosal absorption enhancer, sodium caprate, significantly improved the permeability of an exemplary local anesthetic agent, tetracaine, across a mucosal epithelial cell monolayer in a dose dependent manner.

### Example 2

### Absorption of Local Anesthetics Across a Respiratory Airway Cell Monolayer

Introduction: Three studies were performed to test the ability of various exemplary mucosal absorption enhancers (a fatty acid, a bile salt, a surfactant, and a phospholipid) to promote transport of three exemplary local anesthetic agents (bupivacaine, lidocaine, and tetracaine) across a 3-dimensional MatTek EpiAirway™ cell monolayer. The EpiAirway™ cell line is derived from normal human respiratory airway cells and models the mucosal tissue in the nasal cavity's respiratory airway, the main site of drug absorption following intranasal administration. The MatTek EpiAirway™ cell line has been well correlated to in vivo human intranasal drug absorption (see, e.g., Chemuturi et al., J. Pharm. Sci. 94: 1976-1985, 2005). It is a well-accepted in vitro model of human nasal drug absorption and is an ideal model for intranasal absorption of local anesthetic agents.

### Materials and Methods:

Bupivacaine HCl (P/N B5274; Lot #BCBF0238V; Formula weight 342.90 g/mol), lidocaine HCl (P/N L5647; Lot #SMKBR6002V; Formula weight 288.81 g/mol), and tetracaine HCl (P/N T7508; Lot #SLBF7604V; Formula weight 300.82 g/mol) were purchased from Sigma. Sodium caprate (P/N C4151; Lot #SLBM3367V; Formula weight 194.25 g/mol), sodium deoxycholate (P/N D6750; Lot #086K0045V; Formula weight 414.55 g/mol), polyoxyethylene 9 lauryl ether (P/N P9641; Lot #MKBS1920V; Formula weight 582.81 g/mol), and lysophosphatidylcholine (P/N L4129; Lot #SLBJ4793V; Formula weight n/a) were purchasd from Sigma.

EpiAirway™ tissues were treated with the excipients (0.5% sodium caprate, 0.5% sodium deoxycholate, 0.5% polyoxyethylene 9 lauryl ether, and 0.5% lysophosphatidylcholine) for 20 min. The excipients were then washed off the EpiAirway™ tissues. Test articles (10 mM Bupivacaine HCl, 10 mM Lidocaine HCl, and 10 mM Tetracaine HCl) in transport buffer (1.98 g/L glucose in 10 mM HEPES, 1× Hank's Balanced Salt Solution without Mg²+ and without Ca²⁺, pH 6.0) were added to triplicate apical donor wells. The receiver wells contained standard transport buffer. The fluorescent marker Lucifer Yellow and atenolol (10 μM) were also included to monitor monolayer integrity at the end of the assay. The plates were incubated at 37° C. for 30 min. An aliquot of the receiver chamber was removed for analysis.

At the end of the incubation period, the donor plate was removed from the receiver plate. The donor and receiver samples were analyzed and quantified by LC-MS/MS (propranolol used as internal LC-MS standard).

To verify the monolayer integrity, aliquots of the cell buffers were analyzed by fluorescence and LC-MS/MS to determine the transport of the impermeable dye Lucifer Yellow and atenolol, respectively.

Data are expressed as permeability (Pₐₚₚ): Pₐₚₚ=(dQ/dt)/C₀A

Where dQ/dt is the rate of permeation, C₀ is the initial concentration of test agent, and A is the area of the monolayer.

For mass spectroscopy method development, the signal was optimized for each compound by electrospray ionization (ESI) in positive ionization mode. An MS2 scan was used to optimize the precursor ion mass, and an SIM scan was used to optimize the fragmenter voltage. Product ion analysis was used to identify the best fragment and collision energy for analysis.

Following MRM method optimization, a test injection was performed and an ionization ranking was assigned indicating the compound's ease of ionization.

Samples were analyzed by LC/MS/MS using an Agilent 6410 mass spectrometer coupled with an Agilent 1200 HPLC and a CTC PAL chilled autosampler, all controlled by MassHunter software (Agilent). After separation on a C18 reverse phase HPLC column (Agilent Zorbax StableBond 3.5 um, 2.1×30 mm) using an acetonitrile-water gradient, peaks were analyzed by mass spectrometry using ESI ionization in MRM mode. Diclofenac was utilized as an analytical internal standard (IS).

### Results:

TABLE 2
Effects of Mucosal Absorption Enhancers on Permeability of a Local
Anesthetic Agent

| Test Article | Excipient | Mean Pₐₚₚ (10⁻⁶ cm s⁻¹) |
|---|---|---|
| | none | 19.2 |
| | 0.5% Sodium Caprate | 116 |
| | 0.5% Sodium Deoxycholate | 218 |
| Bupivacaine | 0.5% Polyoxyethylene 9 | 189 |
| | Lauryl Ether | |
| | 0.5% | 173 |
| | Lysophosphatidylcholine | |
| Lidocaine | none | 8.6 |
| | 0.5% Sodium Caprate | 28.9 |
| | 0.5% Sodium Deoxycholate | 96.3 |
| | 0.5% Polyoxyethylene 9 | 71.9 |
| | Lauryl Ether | |
| | 0.5% | 60.8 |
| | Lysophosphatidylcholine | |
| Tetracaine | none | 10.8 |
| | 0.5% Sodium Caprate | 56.8 |
| | 0.5% Sodium Deoxycholate | 155 |
| | 0.5% Polyoxyethylene 9 | 126 |
| | Lauryl Ether | |
| | 0.5% | 71.6 |
| | Lysophosphatidylcholine | |

Pₐₚₚ: apparent permeability rate coefficient;
Assay Duration: 0.5 hr

Lucifer Yellow and atenolol transport met quality control criteria for acceptable monolayer integrity at the end of the assay.

### Conclusions:

Surprisingly, each of the exemplary mucosal absorption enhancers significantly increased the absorption of each of the exemplary local anesthetics used in these three studies. This discovery demonstrates the novel utility of using mucosal absorption enhancers to promote intranasal transmucosal absorption of local anesthetic agents in order to achieve anesthesia of the trigeminal nerve pathway.

### Example 3

### Local Anesthetic Absorption Across an Epithelial Cell Monolayer

An experiment is performed to test the ability of a mucosal absorption enhancer to promote transport of a local anesthetic agent across an epithelial cell monolayer. In this experiment, the Caco-2 cell line (an intestinal mucosal cell line) is grown and maintained at a pH at 6.5 for a length of time sufficient to establish tight junction between cells in the monolayer. After formation of the monolayer, a pharmaceutical composition (e.g., the pharmaceutical composition shown in Table 3, Table 8, or Table 11) is administered to one side of the Caco-2 cell monoloayer. A pharmaceutical composition containing tetracaine but lacking a mucosal absorption enhancer is administered to one side of a separate Caco-2 cell monolayer as a control. The quantity of tetracaine that is able to undergo absorption (e.g., paracellular transport and/or transcellular transport) across the Caco-2 cell monolayer is quantified using HPLC analysis, and compared to the level of absorption (e.g., paracellular transport and/or transcellular transport) achieved in a composition that contains tetracaine but is absent of a mucosal absorption enhancer. Drug transport across Caco-2 cell monolayers is a well-known model of transmucosal drug absorption in humans.

### Example 4

### Determination of Mucoadhesive Properties

The Mucin Particle Method is a simple assay that indicates whether or not a sample agent has mucoadhesive properties. Mucin particles (e.g., commercially available porcine mucin particles) are suspended in a suitable buffer with a concentration of 1% (w/v). The sample agent (e.g., chitosan hydrochloride) that is to be tested for mucoadhesive properties is added to the solution at a controlled concentration. The zeta potential of the mucin particle solution is tested before and after the addition of the sample agent, and the values are then compared. Change in the measured zeta potential indicates mucoadhesive properties.

### Example 5

### Local Anesthetic Absorption Along the Trigeminal Nerve Pathway in Rats

A control composition that contains a local anesthetic agent without a mucosal absorption enhancer and one of the pharmaceutical compositions provided herein (e.g., the exemplary composition of Table 3, 7, or 11) is intranasally administered to a set number of rats. The local anesthetic agent in each tested composition is radiolabeled. The absorption of the labeled local anesthetic agent into the trigeminal nerve pathway and various tissues innervated by the trigeminal nerve pathway is monitored and quantified by microPET imaging. Improved transmucosal absorption of a local anesthetic agent is determined by comparing the quantity of drug transported across the nasal epithelium and/or absorption into target tissues.

### Example 6

### Determining Effectiveness in Human Subjects

A control composition that contains a local anesthetic agent without a mucosal absorption enhancer and one of the pharmaceutical compositions provided herein (e.g., the exemplary composition of Table 3, 7, or 11) is intranasally administered to a set number of human person(s). An electric pulp test (EPT) is utilized to quantify pain and anesthesia of the teeth. The EPT is utilized in such a way wherein the lowest level of current is a “0” and the highest level of current is a “80.” An increasing amount of current is exposed to the subject and the current is stopped when the subject feels any pain. The value of the current is then recorded. Anesthesia is considered successful when a subject does not report any pain at a current value of “80.” The subject(s) maxillary teeth (e.g., maxillary molars) and/or mandibular teeth (e.g., mandibular molars) are tested with the control composition and one of the pharmaceutical compositions provided herein to determine improved anesthesia. EPT experiments are a well-known method for determining tooth pain and are utilized in identifying pulpal necrosis or dental abscess.

### Example 7

### Exemplary Pharmaceutical Composition

A nasal deliverable anesthetizing pharmaceutical composition is prepared by the following ingredients as specified in Table 3.

TABLE 3
Exemplary Pharmaceutical Composition

| Ingredient | Formulation (% w/v) |
|---|---|
| Tetracaine hydrochloride, USP | 3.00 |
| Chitosan hydrochloride, USP | 0.50 |
| Oxymetazoline hydrochloride, USP | 0.05 |
| Citric acid anhydrous, USP | 1.00 |
| Sodium hydroxide, NF | q.s. |
| Benzyl alcohol, NF | 0.90 |
| Hydrochloric acid, NF | q.s. |
| Purified water, USP | q.s. to 100% |

### Example 8

### Exemplary Pharmaceutical Composition

A nasal deliverable anesthetizing pharmaceutical composition is prepared by the following ingredients as specified in Table 4.

TABLE 4
Exemplary Pharmaceutical Composition

| Ingredient | Formulation (% w/v) |
|---|---|
| Bupivacaine hydrochloride, USP | 2.00 |
| Sodium deoxycholate | 0.25 |
| Oxymetazoline hydrochloride, USP | 0.05 |
| Citric acid anhydrous, USP | 1.00 |
| Sodium hydroxide, NF | q.s. |
| Benzyl alcohol, NF | 0.90 |
| Hydrochloric acid, NF | q.s. |
| Purified water, USP | q.s. to 100% |

### Example 9

### Exemplary Pharmaceutical Composition

A nasal deliverable anesthetizing pharmaceutical composition is prepared by the following ingredients as specified in Table 5.

TABLE 5
Exemplary Pharmaceutical Composition

| Ingredient | Formulation (% w/v) |
|---|---|
| Lidocaine hydrochloride, USP | 4.00 |
| Lysophosphatidylcholine, USP | 0.50 |
| Oxymetazoline hydrochloride, USP | 0.05 |
| Citric acid anhydrous, USP | 1.00 |
| Sodium hydroxide, NF | q.s. |
| Benzyl alcohol, NF | 0.90 |
| Hydrochloric acid, NF | q.s. |
| Purified water, USP | q.s. to 100% |

### Example 10

### Exemplary Pharmaceutical Composition

A nasal deliverable anesthetizing pharmaceutical composition is prepared by the following ingredients as specified in Table 6.

TABLE 6
Exemplary Pharmaceutical Composition

| Ingredient | Formulation (% w/v) |
|---|---|
| Mepivacaine hydrochloride, USP | 5.00 |
| Polyoxyethylene 9 lauryl ether | 0.50 |
| Oxymetazoline hydrochloride, USP | 0.05 |
| Citric acid anhydrous, USP | 1.00 |
| Sodium hydroxide, NF | q.s. |
| Benzyl alcohol, NF | 0.90 |
| Hydrochloric acid, NF | q.s. |
| Purified water, USP | q.s. to 100% |

### Example 11

### Exemplary Pharmaceutical Composition

A nasal deliverable anesthetizing pharmaceutical composition is prepared by the following ingredients as specified in Table 7.

TABLE 7
Exemplary Pharmaceutical Composition

| Ingredient | Formulation (% w/v) |
|---|---|
| Tetracaine hydrochloride, USP | 3.00 |
| Sodium caprate | 0.25 |
| Oxymetazoline hydrochloride, USP | 0.05 |
| Citric acid anhydrous, USP | 1.00 |
| Sodium hydroxide, NF | q.s. |
| Benzyl alcohol, NF | 0.90 |
| Hydrochloric acid, NF | q.s. |
| Purified water, USP | q.s. to 100% |

### Example 12

### Exemplary Pharmaceutical Composition

A nasal deliverable anesthetizing pharmaceutical composition is prepared by the following ingredients as specified in Table 8.

TABLE 8
Exemplary Pharmaceutical Composition

| Ingredient | Formulation (% w/w) |
|---|---|
| Mepivacaine base, USP | 2.00 |
| Beta-cyclodextrin, USP | 2.00 |
| Purified water, USP | q.s. to 100% |

### Example 13

### Exemplary Pharmaceutical Composition

A liposomal nasal deliverable anesthetizing pharmaceutical composition is prepared by the following ingredients as specified in Table 9.

TABLE 9
Exemplary Liposomal Pharmaceutical Composition

| Ingredient | Formulation (% w/v) |
|---|---|
| Tetracaine base, USP | 3.00 |
| DOPC | 7.00 |
| Cholesterol, USP | 1.75 |
| Citric acid anhydrous, USP | 1.00 |
| Benzyl alcohol, NF | 0.90 |
| Sodium Hydroxide, NF | q.s. |
| Hydrochloric acid, NF | q.s. |
| Purified water, USP | q.s. to 100% |

### Example 14

### Exemplary Pharmaceutical Composition

A nasal deliverable anesthetizing pharmaceutical composition is prepared by the following ingredients as specified in Table 10.

TABLE 10
Exemplary Pharmaceutical Composition

| Ingredient | Formulation (% w/w) |
|---|---|
| Bupivacaine base, USP | 5.00 |
| Propylene glycol, USP | q.s. to 100% |

### Example 15

### Exemplary Pharmaceutical Composition

A microemulsion nasal deliverable anesthetizing pharmaceutical composition is prepared by the following ingredients as specified in Table 11.

TABLE 11
Exemplary Pharmaceutical Composition

| Ingredient | Formulation (% w/w) |
|---|---|
| Tetracaine hydrochloride, USP | 5.00 |
| Castor oil, USP | 5.00 |
| Polysorbate 80, USP | 10.00 |
| Propylene glycol, USP | 20.00 |
| Purified water, USP | 60.00 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of locally and specifically anesthetizing nerve tissue in the trigeminal nerve pathway in a subject in need thereof, the method comprising:
intranasally administering a pharmaceutical composition comprising a local anesthetic agent and a mucosal absorption enhancer to a subject in need thereof, wherein:
the local anesthetic agent and mucosal absorption enhancer are present in an amount sufficient to anesthetize nerve tissue in the trigeminal nerve pathway upon administration.

2. The method of claim 1, wherein the nerve tissue in the trigeminal nerve pathway comprises one or more of trigeminal ganglion, the frontal nerve, the supraorbital nerve, the supratrochlear nerve, the lacrimal nerve, the nasociliary nerve, the infratrochlear nerve, the ciliary nerve, the anterior ethmoidal nerve, the external nasal nerve, the internal nasal nerve, the ophthalmic nerve (V1), the infraorbital nerve, the anterior superior alveolar nerve, the middle superior alveolar nerve, the infraorbital nerve entering the infraorbital canal, the posterior superior alveolar nerve, the ganglionic branches to pterygopalatine ganglion, the pterygopalatine ganglion, the greater and lesser palantine nerves, the lateral nasal branches of the greater palatine nerve, nerve of the pterygoid canal, the nasopalatine nerve, the zygomatic nerve, the zygomaticofacial nerve, the zygomaticotemporal nerve, the lateral nasal branches of the maxillary nerve, the maxillary nerve (V2), the buccal nerve, the auriculotemporal nerve, the lingual nerve, the inferior alveolar nerve, the mylohyoid nerve, the mental nerve, the incisive nerve, and the mandibular nerve (V3).

3. The method of claim 1, wherein the pharmaceutical composition is directly administered onto one or more of the following nasal tissues: the inferior nasal turbinate, the middle nasal turbinate, the nasal meatuses, the extreme rear of the nasal cavity, and the maxillary sinus.

4. The method of claim 1, wherein the local anesthetic agent is a free-base form of the local anesthetic agent.

5. The method of claim 1, wherein the local anesthetic agent is selected from the group consisting of: benzocaine, butacaine, tetracaine, lidocaine, dyclonine, pramoxine, dibucaine, cocaine, etidocaine, bupivacaine, levobupivacaine, ropivacaine, procaine, chloroprocaine, mepivacaine, prilocaine, articaine, hexylcaine, oxetacaine, and a salt thereof

6. The method of claim 1, wherein the local anesthetic agent comprises about 0.1% to about 50% by weight of the pharmaceutical composition.

7. The method of claim 1, wherein the mucosal absorption enhancer is selected from the group consisting of: a monosaccharide, a polysaccharide, a bile salt, a surfactant, an oil, a fusidate compound, a cyclodextrin, a phospholipid, a thiomer, a fatty acid, a chelator, a salicylate, a polymer, a micelle, an alcohol, a liposome, a microemulsion, an emulsion, a solid lipid nanoparticle, a transferosome, propylene glycol, menthol, ammonium glycyyhizinate, glycrrhetinic acid, aminated gelatin, laurocapram, benzalkonium chloride, a phenothiazine, a nitric acid donor, zonula occluden toxin, a poly-L-arginine, a soybean derivative glucoside, citicholine, and an alpha-acid derivative.

8. The method of claim 1, wherein the mucosal absorption enhancer comprises about 0.001% to about 50% by weight of the pharmaceutical composition.

9. The method of claim 7, wherein the mucosal absorption enhancer is a liposome or a solid lipid nanoparticle.

10. The method of claim 7, wherein the mucosal absorption enhancer is propylene glycol.

11. The method of claim 7, wherein the mucosal absorption enhancer is a microemulsion.

12. The method of claim 11, wherein the microemulsion is an oil-in-water microemulsion comprising an aqueous phase, a lipid phase, and a surfactant.

13. The method of claim 12, wherein the microemulsion further comprises a co-surfactant.

14. The method of claim 12, wherein the aqueous phase comprises at least about 10% by weight of the pharmaceutical composition.

15. The method of claim 12, wherein the lipid phase comprises about 0.1% to about 50% by weight of the pharmaceutical composition.

16. The method of claim 13, wherein one or both of the surfactant and the co-surfactant each comprises about 0.1% to about 80% by weight of the pharmaceutical composition.

17. The method of claim 12, wherein the lipid to total surfactant ratio in the pharmaceutical composition is between about 1:1 to about 1:10.

18. The method of claim 13, wherein the surfactant to co-surfactant ratio in the pharmaceutical composition is between about 6:1 to about 1:6.

19. The method of claim 12, wherein the lipid is selected from the group consisting of: castor oil, corn oil, cottonseed oil, olive oil, peanut oil, soybean oil, vegetable oil, coconut oil, isopropyl palmitate, light mineral oil, oleic acid, medium chain mono/diglycerides, and propylene glycol dicaprylocaprate.

20. The method of claim 12, wherein the lipid is a local anesthetic agent in its oil form.

21. The method of claim 13, wherein the surfactant and/or the co-surfactant is selected from the group consisting of: polysorbate 20, polysorbate 40, polysorbate 60, polysorbate 65, polysorbate 80, polyoxyl 8 stearate, polypropylene glycol, propylene glycol, carbomer 1342, carbomer 934, carbomer 934P, carbomer 940, carbomer 941, carbomer 974, carbomer 980, carbomer 981, oleyl polyethylene glycol glyceride, PEG-300, PEG-400, PEG-3500, poloxamer 124, poloxamer 188, poloxamer 407, polyoxyl 35 castor oil, polyoxyl 40 hydrogenated castor oil, polyoxyl 40 stearate, polyoxyl 6 and polyoxyl 32 palmitostearate, polyoxyl 8 stearate, polyoxyl glyceryl stearate, polyoxyl lanolin, polyoxyl palmitate, polyoxyl stearate, glyceryl oleate, propylene carbonate, sorbitan monooleate, glycerin, PEG-5 oleate, sodium N-methyl N-cocoyl taurate, PEG-8 caprylic/capric gylcerides, and diethylene glycol monoethyl ether.

22. The method of claim 1, wherein the pharmaceutical composition further comprises a viscosity enhancing agent.

23. The method of claim 1, wherein the viscosity enhancing agent is selected from the group consisting of: hydroxyethylcellulose, hydroxypropylmethyl cellulose, sodium carboxy methyl cellulose, carbomer homopolymer type a, carbomer homopolymer type b, carbomer, polycarbophil, sodium alginate, xantham gum, smart hydrogel, polyethylene glycol, hydroxycellulose, poloxamer 188, poloxamer 407, starch, aminated gelatin, chitosan, hydroxypropyl methylcellulose, hydroxypropyl cellulose, methylcellulose, carboxymethyl cellulose, ethylcellulose, and microcrystalline cellulose.

24. The method of claim 22, wherein the viscosity enhancing agent comprises about 0.01% to about 10% by weight of the pharmaceutical composition.

25. The method of claim 22, wherein the pharmaceutical composition further comprises a vasoconstrictor.

26. The method of claim 25, wherein the vasoconstrictor is selected from the group consisting of: oxymetazoline, phenylephrine, naphazoline, propylhexadrine, levodesoxyephedrine, epinephrine, norepinephrine, and a salt thereof

27. The method of claim 22, wherein the vasoconstrictor comprises about 0.001% to about 1% by weight of the pharmaceutical composition.

28. The method of claim 1, wherein the subject is in need of oral surgery or a dental procedure.

29. The method of claim 1, further comprising:
making at least one incision, puncture, or suture in at least one tissue selected from the group consisting of: dental pulp, soft tissue in the oral cavity, eye, eye socket, eyelid, soft tissue of the face, and sinuses.

30. The method of claim 29, wherein the subject is in need of oral surgery and the nerve tissue is one or more of anterior superior alveolar nerve, middle superior alveolar nerve, infraorbital nerve entering the infraorbtial canal, posterior superior alveolar nerve, maxillary nerve, mandibular nerve, and trigeminal nerve.
